(19) United States
(12) Patent Application Publication
(10) Pub. No.: US 2020/0140560 A1
(43) Pub. Date: May 7, 2020

(54) PROTEASE BASED SWITCH CHIMERIC ANTIGEN RECEPTORS FOR SAFER CELL IMMUNOTHERAPY

(71) Applicant: CELLECTIS, Paris (FR)

(72) Inventors: Philippe Duchateau, Draveil (FR); Alexandre Juillerat, New York, NY (US); Laurent Poirot, Paris (FR)

(21) Appl. No.: 16/612,280

(22) PCT Filed: May 11, 2018
(86) PCT No.: PCT/EP2018/062253

§ 371 Date: Nov. 8, 2019

Related U.S. Application Data
(60) Provisional application No. 62/505,528, filed on May 12, 2017.

(30) Foreign Application Priority Data
May 29, 2017 (DK) PA201770396

(51) Int. Cl. C07K 16/28; C07K 14/725; C12N 9/50

(52) U.S. Cl. CPC C07K 16/2866; C07K 14/7051; C12N 9/50; C07K 2317/53; C07K 2319/03; C07K 2319/02; C07K 2317/24

(57) ABSTRACT

The present invention relates to the field of cell immunotherapy and more particularly to a new generation of chimeric antigen receptors (CAR). These new CARs are primarily expressed into cells under the form of chimeric polypeptide precursors that can be made active by a protease and switched-off upon addition of a protease inhibitor. Once activated by the protease, such CARs reach the surface of the immune cells and bind specific antigens. More specifically, the presentation of these CARs at the cells' surface is made controllable by inclusion in their polypeptide structure of a protease domain and/or a degradation domain (e.g. degron).

### FIELD OF THE INVENTION

The present invention relates to the field of cell immunotherapy and more particularly to a new generation of chimeric antigen receptors (CAR). These new CARs are primarily expressed into cells under the form of chimeric polypeptide precursors that can be made active by a protease. Once activated they reach the surface of the immune cells and bind specific antigens. More specifically, the presentation of these CARs at the cells' surface is made controllable by inclusion in their polypeptide structure of a protease domain and/or a degradation domain (e.g. degron). Such domains can prevent the presentation of the CAR at the cell surface and be excised under certain conditions, such as the presence or absence of a small molecule (e.g.: protease inhibitor), preferably an approved drug. The invention thereby provides with various CAR architectures sensitive to small molecules that can easily penetrate cells. 20 These new chimeric polypeptides are used to endow engineered immune cells, such as NK or T-lymphocytes, for a safer therapeutic use thereof. The methods of the present invention may also apply to recombinant T-cell receptors (TCR).

### BACKGROUND OF THE INVENTION

Adoptive immunotherapy, which involves the transfer of autologous or allogeneic antigen-specific immune cells generated ex vivo, is a promising strategy to treat viral infections and cancer [Poirot, L. et al. (2015) Multiplex Genome-Edited T-cell Manufacturing Platform for “Off-the-Shelf” Adoptive T-cell Immunotherapies. Cancer Res. 75(18)]. The immune cells generally used for adoptive immunotherapy can be generated by expansion of antigen-specific T cells or NK cells [Chu, J. et al. (2014) CS1-specific chimeric antigen receptor (CAR)-engineered natural killer cells enhance in vitro and in vivo antitumor activity against human multiple myeloma. Leukemia 28:917-927]. The potential of this approach relies on the ability to redirect the specificity of T cells through genetic engineering and transfer of chimeric antigen receptors (CARs) or engineered TCRsl. Numerous clinical studies have demonstrated the potential of adoptive transfer of CAR T cells for cancer therapy. However some raised concerns with the risks associated with the so-called cytokine-release syndrome (CRS) and the “on-target off-tumor” effect [Morgan, R. A. et al. (2010) Case report of a serious adverse event following the administration of T cells transduced with a chimeric antigen receptor recognizing ERBB2. Mol Ther 18:843-851].

To date, few strategies have been developed to pharmacologically control CAR engineered T-cells. Current strategies mainly rely on suicide mechanisms [Marin, V. et al. (2012) Comparison of different suicide-gene strategies for the safety improvement of genetically manipulated T cells. Hum Gene Ther Methods 23:376-386]. Such suicide strategies aim to a complete eradication of the engineered T-cells, which will result in the premature end of the treatment. Thus, implementing non-lethal control of engineered CAR T-cells could represent an important advancement to improve the CAR T-cell technology and its safety.

Small molecule based approaches that rely on dimerizing partner proteins have already been used to study, inter alia, the mechanism of T-cell receptor triggering [James, J. R. et al. (2012) Biophysical mechanism of T-cell receptor triggering in a reconstituted system. Nature. 487: 64-69]. Recently, Lim et al. have adapted this approach to control engineered T-cells through the use of a multichain receptor [Remote control of therapeutic T cells through a small molecule-gated chimeric receptor. Science (2015) Vol. 350 (6258)].

Here, the inventors have set up a strategy to create controllable engineered CAR T-cells, which may be implemented on single-chain as well as multi-chain CARs. Their approach is based on classical CAR architectures in which they have introduced degradation domains, such as degrons, promoting intracellular degradation of the CARS through the proteasome. This degradation is placed under the dependency of an approved drug compound, so that the CAR presentation at the surface of the cells can be modulated in-vivo through the administration of said drug.

By controlling scFv presentation at the cell surface upon expression of these new architectures of CARs (degron CARs), the inventors have shown that they could induce or stop the cytolytic properties of the engineered T-cell in-vivo through various doses of the drug compound. Overall, this non-lethal system offers the advantage of providing “transient CAR T-cell”, thereby improving their safety and therapeutic activity (reducing immune cells exhaustion).

### SUMMARY OF THE INVENTION

The present invention is drawn to new chimeric polypeptides and related polynucleotides that are expressible in immune cells and which can be regarded as precursors of chimeric antigen receptors (CAR) aiming at being presented at the surface of said immune cells. Such chimeric polypeptides typically comprise a first polypeptide encoding a CAR linked to a second polypeptide encoding a protease that has the ability to induce cleavage of said chimeric polypeptide. Upon cleavage by the protease, a functional CAR is released, which can sit at the surface of the immune cells permitting the activation of said immune cells upon interaction with specific antigens.

According to certain embodiments of the invention, the protease comprised into the chimeric polypeptide can be inhibited by a protease inhibitor. In such an event, the CAR is not necessary cleaved by the protease and remains inactive or weakly active. The presentation of the CAR at the surface of the immune cells can then be reduced or put on hold by maintaining the engineered cells in contact with a dose of said protease inhibitor as long as required (switch-off configuration). In the opposite, if a CAR is designed with a cleavage site recognized by a protease which is co-expressed into the cell, then administration of the protease inhibitor could reduce cleavage of the CAR polypeptide, thereby allowing its presentation at the surface of the immune cells (switch-on configuration).

The invention also provides with chimeric polypeptides comprising a degron—a polypeptide sequence recognized by the proteasome, which directs the intracellular degradation of the CARs. Such degrons, which are included into the chimeric polypeptide of the invention, can induce the degradation of the CAR by the proteasome, with the effect of reducing or impairing the presentation of the CAR at the surface of the cells. Hence, a reduced activation of the immune cells expressing the chimeric polypeptides can be obtained.

Still according to the invention, the chimeric polypeptides can comprise both a degron and a protease domain to enhance control on the CAR polypeptide. According to certain embodiments, the degron is preferably included into a self-excision domain. In a preferred embodiment, the degron is located into a self-excision domain that encodes a protease. An example of such a protease is the nonstructural protein 3 (NS3) protease, the activity of which can be reduced or inhibited by a protease inhibitor, such as asunaprevir, simeprevir, danoprevir or ciluprevir.

The chimeric polypeptides according to the present invention, which comprise a protease and/or a degron can display different structures as further detailed in this application.

The invention also relates to the polynucleotides encoding the above polypeptides, especially for their insertion into immune cell's genome, more preferably at the TCR locus of T-cells or NK-cells. Such insertion at this locus can lead to the inactivation or lower expression of TCR, making such engineered cells less alloreactive.

The invention also encompasses methods of expressing such chimeric polypeptides into immune cells to create engineered immune cells to be used in cell therapy, methods of treating patients with such engineered immune cells, either as part of allogeneic or autologous treatments, and methods of infusing patients with same in combination with protease inhibitors to control CAR's expression at the surface of the immune cells, and in-fine, obtaining better control of their therapeutic activity.

### BRIEF DESCRIPTION OF THE FIGURES

FIG. 1: Schematic representation of a degron CARs of the present invention and principle of use. The CAR comprises in its architecture a degradation moiety controllable by a small molecule (e.g.: protease inhibitor) that includes a degron. In the presence of the small molecule, the degradation moiety is not functional and the degron induces intracellular degradation of the CAR by the proteasome. In the absence of the small molecule, a protease activity is expressed and the degron is cleaved off the CAR. The functional CAR is not degraded by the proteasome and can present its external binding domain (e.g. ScFv) at the surface of the T-cells. Hence, the CAR becomes active and can activate the T-cells.

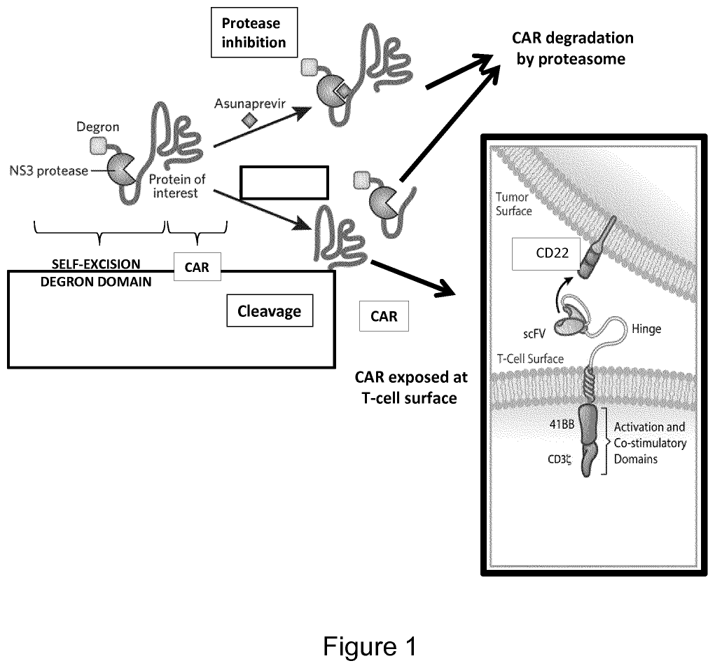

FIG. 2: Schematic representation featuring the principle of the invention to obtain therapeutic immune cells endowed with CAR that can be switched-off upon addition in the culture medium or administration into the patient of a protease inhibitor, such as Asunaprevir. The CAR is referred to as SWOFF-CAR (Switch-off Chimeric Antigen Receptor) A: In the absence of protease inhibitor the CAR is expressed, cleaved off the degron, and normally presented at the surface of the immune cell. B: in the presence of the protease inhibitor, the CAR is not separated from the degron and is entirely processed for degradation through the proteasome.

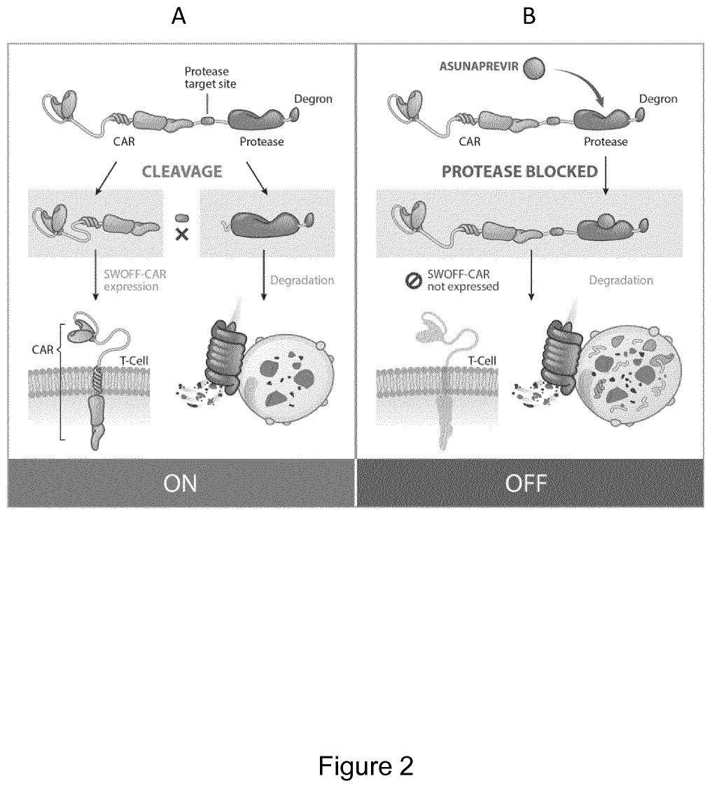

FIG. 3: Schematic representation of the drug-dependent and antigen-dependent CAR immune cells activation as per the CAR system of the present invention (e.g.: “AND GATE” that requires the absence of drug and the presence of a specific antigen to transduce activation signal).

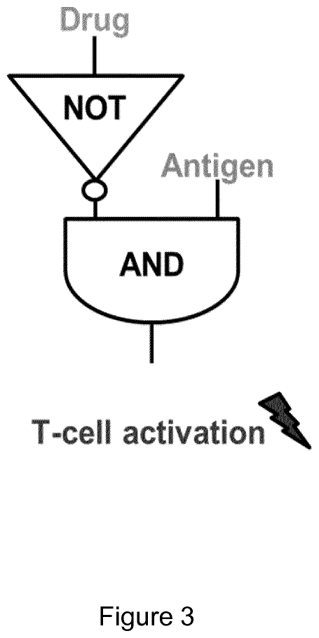

FIG. 4: Examples of architectures of CARs with small molecule controlled degradation according to the present invention. 4A: CARs with N-terminal self-excision degron. 4B: CARs with C-terminal self-excision degron (sequence details are given in example 1).

FIG. 5: Further examples of CARs architectures enabling small molecule based control activation according to the invention.

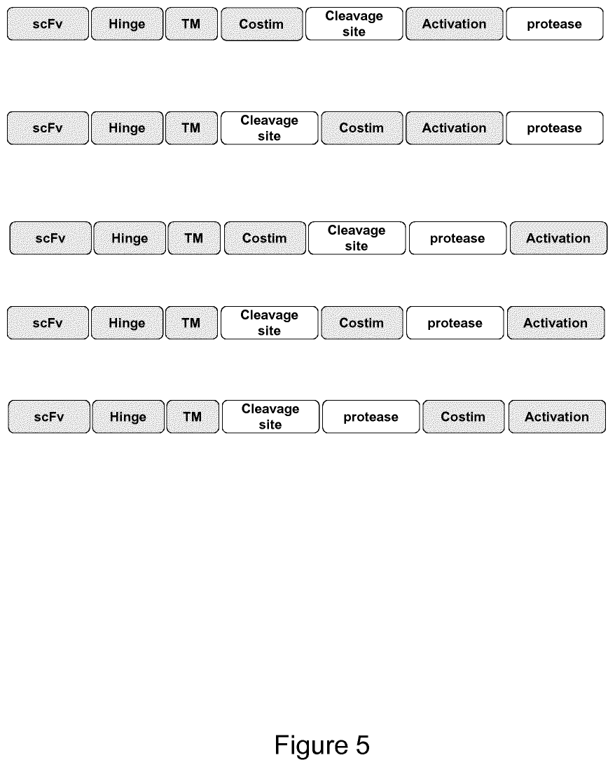

FIG. 6: Experimental results obtained with T-cells endowed with the CARs of the present invention. 6A: Percentage of CAR positive T-cells (presentation of anti-CD123 CARs at the surface of the transduced cells) in presence or absence of the protease inhibitor Asunaprevir. 6B: Percentage of CAR positive T-cells (presentation of anti-CD22 CARs at the surface of the transduced cells) in presence or absence of the protease inhibitor Asunaprevir). Controls are T-cells endowed with CARs lacking controlled degradation moiety (high presentation of CARs at the surface of the transduced cells). The percentage of CAR positive cells is measured by flow cytometry. Experimental details are provided in example 2.

FIG. 7: Percentage of CD22 positive target cells killed by the T-cells engineered according to the invention endowed with a CAR comprising a controlled degradation moiety in presence (+ASN) and absence (−ASN) of Asunaprevir. The percentage of killed cells is reduced by the addition of 500 nM Asunaprevir in the three experiments. Data are normalized using untransduced human primary T-cells. Experimental details are provided in example 3.

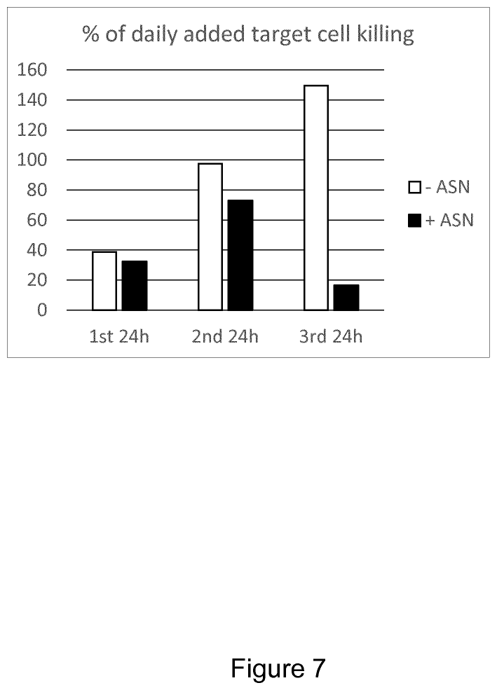

FIG. 8: Cytotoxicity assays performed against CD22 positive Raji cells—Raji cells were incubated with the CAR anti-CD22 T-cells according to the invention at D5 and D6, while the % of Raji cells killed by the CAR anti-CD22 T-cells was measured at periods 0-24 h and 24-48 h in presence (adjunction of 500 mM ASN stopped at D3, D4, D5 and D6) or absence (no drug) of Asunaprevir. 8A: % of CD22 positive cells killed over the first period 0-24 h. 8B: % of CD22 positive cells killed over the second period 24-48 h.

FIG. 9: Proliferation of T-cells in the presence of increasing concentrations of Asunaprevir (see example 5). The total number of cells at different days cultured in presence of 100 nM, 500 nM or 1000 nM relative to 0 nM ASN is presented. Data are shown as the median of PBMC from 2 donors done in duplicate.

FIG. 10: Cytokine quantification after co-culture of anti-CD22 CAR T-cells with target cells as a function of Asunaprevir concentration (see example 6). Data are shown as the mean±SD of duplicates per points.

FIG. 11: MFI (CAR detection) of primary T-cells transduced with an engineered CAR in the absence (white bars) or presence of 500 nM Asunaprevir (dark gray, two different providers) as further detailed in Example 7.

FIG. 12: Schematic representation of the donor template and TRAC locus according to the present invention as used in Example 8 herein.

FIG. 13: Flow cytometry analysis of engineered CAR surface expression upon TCRα/β knockout (insertion of the exogenous sequence encoding CAR at the TCR locus) as further detailed in Example 8.

FIG. 14: Luciferase signal (target cells) measured at the end of the assay detailed in Example 8 (the signal is normalized to the highest value of each replicates). Data are shown as median with 95% confidence intervals of triplicates per points. N=2, performed in triplicates.

FIG. 15: Fitting of the normalized luciferase signal with respect to ASN concentration showing that luciferase signal is significant at therapeutically acceptable ASN concentrations.

### DETAILED DESCRIPTION OF THE INVENTION

Unless specifically defined herein, all technical and scientific terms used have the same meaning as commonly understood by a skilled artisan in the fields of gene therapy, biochemistry, genetics, and molecular biology.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Frederick M. AUSUBEL, 2000, Wiley and son Inc, Library of Congress, USA); Molecular Cloning: A Laboratory Manual, Third Edition, (Sambrook et al, 2001, Cold Spring Harbor, N.Y.: Cold Spring Harbor Laboratory Press); Oligonucleotide Synthesis (M. J. Gait ed., 1984); Mullis et al. U.S. Pat. No. 4,683,195; Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); and Manipulating the Mouse Embryo, (Cold Spring Harbor Laboratory Press, Cold Spring Harbor, N.Y., 1986).

The present invention is primarily drawn to chimeric polynucleotides, encoding chimeric polypeptides, to be heterologously expressed in effector immune cells under the form of chimeric antigen receptors (CAR) or artificial T-cell receptors (also called “recombinant TCR”).

The chimeric polypeptide according to the present invention are preferably expressed under the form of “conditional” chimeric antigen receptors controllable by drugs. The effect of the drug can be positive (i.e.—leading to activation of the CAR=“switch on” effect) or negative (i.e. leading to inhibition of the activation of the CAR=“switch off” effect), depending on the design of the chimeric polypeptide as further detailed in this application.

Such chimeric polypeptide according to the invention is characterized in that it comprises a protease and/or a degron polypeptide domain, preferably both of them, and more preferably in such a way that the protease and the degron domains can be excised from the chimeric polypeptide to release a functional effector transmembrane polypeptide.

By “drug” is meant a small molecule, preferably approved for human administration, which can penetrate the immune cells in view of interacting with the above chimeric polypeptide.

By “chimeric polynucleotide or polypeptide” is meant a single chain polynucleotide or polypeptide structure, comprising different polynucleotide coding sequences or polypeptide sequences. Said chimeric polynucleotide or polypeptide according to the invention can comprises an effector polypeptide, preferably a chimeric antigen receptor or a recombinant T-cell receptor.

By ‘effector polypeptide” is meant any transmembrane polypeptide, generally a protein or peptide molecule that provides a benefit to hosts in the context of infection, predation or competition, preferably a receptor or a component thereof, which transduces an external signal into the cell to activate some of its functionality(ies).

By “chimeric antigen receptor” are synthetic receptors consisting of an external targeting moiety that is associated with one or more signaling domains in a single fusion polypeptide. In general, the binding moiety of a CAR consists of an antigen-binding domain of a single-chain antibody (scFv), comprising the light and heavy variable fragments of a monoclonal antibody joined by a flexible linker. Binding moieties based on receptor or ligand domains have also been used successfully. The signaling domains for first generation CARs are derived from the cytoplasmic region of the CD3zeta or the Fc receptor gamma chains. First generation CARs have been shown to successfully redirect T cell cytotoxicity, however, in order to provide prolonged expansion and anti-tumor activity in vivo, signaling domains from co-stimulatory molecules including CD28, OX-40 (CD134), ICOS and 4-1BB (CD137) have been added alone (second generation) or in combination (third generation) to enhance survival and increase proliferation of CAR modified T cells.

By “recombinant T-cell receptor” is meant an artificially modified T-cell receptor in which at least one of its components is obtained by expression of exogenous polynucleotide. The intracellular signalling domain of recombinant can be derived from the cytoplasmic part of a membrane bound receptor to induce cellular activation, e.g., the Fc epsilon RI receptor gamma-chain or the CD3 zeta-chain. By use of this type of recombinant receptor, one can combines the advantages of MHC-independent, antibody-based antigen binding with efficient T cell activation upon specific binding to the receptor ligand. This approach can be regarded as an alternative to CARs for the engineering of antigen-specific T-cells for immunotherapy [Hombach, A. et al. (2002) The recombinant T cell receptor strategy: insights into structure and function of recombinant immunoreceptors on the way towards an optimal receptor design for cellular immunotherapy. Curr Gene Ther. 2(2):211-26]. A component of such T-cell receptor can be linked to a protease or a degron polypeptide domain to form a chimeric polynucleotide or polypeptide according to the present invention.

Expressing chimeric antigen receptors (CAR) or recombinant T-cell receptors have become the state of the art to direct or improve the specificity of primary immune cells, especially in T-cells for treating tumors or infected cells. CARs expressed in such immune cells, by specifically targeting antigen markers, helps said immune cells to destroy malignant of infected cells in-vivo (Sadelain M. et al. “The basic principles of chimeric antigen receptor design” (2013) Cancer Discov. 3(4):388-98). CARs are usually designed to include activation domains that stimulate immune cells in response to binding to a specific antigen (so-called positive CAR), but they may also comprise an inhibitory domain with the opposite effect (so-called negative CAR)(Fedorov, V. D. (2014) “Novel Approaches to Enhance the Specificity and Safety of Engineered T Cells” Cancer Journal 20 (2):160-165. Positive and negative CARs may be combined or co-expressed to finely tune the cells immune specificity depending of the various antigens present at the surface of the target cells.

Preferred examples of signal transducing domain for use in a CAR can be the cytoplasmic sequences of the T cell receptor and co-receptors that act in concert to initiate signal transduction following antigen receptor engagement, as well as any derivate or variant of these sequences and any synthetic sequence that has the same functional capability. Signal transduction domain comprises two distinct classes of cytoplasmic signaling sequence, those that initiate antigen-dependent primary activation, and those that act in an antigen-independent manner to provide a secondary or co-stimulatory signal. Primary cytoplasmic signaling sequence can comprise signaling motifs which are known as immunoreceptor tyrosine-based activation motifs of ITAMs. ITAMs are well defined signaling motifs found in the intracytoplasmic tail of a variety of receptors that serve as binding sites for syk/zap70 class tyrosine kinases. Examples of ITAM used in the invention can include as non-limiting examples those derived from TCRzeta, FcRgamma, FcRbeta, FcRepsilon, CD3gamma, CD3delta, CD3epsilon, CD5, CD22, CD79a, CD79b and CD66d. In a preferred embodiment, the signaling transducing domain of the CAR can comprise the CD3zeta signaling domain which has amino acid sequence with at least 70%, preferably at least 80%, more preferably at least 90%, 95% 97% or 99% sequence identity with amino acid sequence selected from the group consisting of (SEQ ID NO: 9).

In particular embodiment the signal transduction domain of the CAR of the present invention comprises a co-stimulatory signal molecule. A co-stimulatory molecule is a cell surface molecule other than an antigen receptor or their ligands that is required for an efficient immune response. “Co-stimulatory ligand” refers to a molecule on an antigen presenting cell that specifically binds a cognate co-stimulatory molecule on a T-cell, thereby providing a signal which, in addition to the primary signal provided by, for instance, binding of a TCR/CD3 complex with an MHC molecule loaded with peptide, mediates a T cell response, including, but not limited to, proliferation activation, differentiation and the like. A co-stimulatory ligand can include but is not limited to CD7, B7-1 (CD80), B7-2 (CD86), PD-L1, PD-L2, 4-1BBL, OX40L, inducible costimulatory ligand (ICOS-L), intercellular adhesion molecule (ICAM, CD30L, CD40, CD70, CD83, HLA-G, MICA, M1CB, HVEM, lymphotoxin beta receptor, 3/TR6, ILT3, ILT4, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as but not limited to, CD27, CD28, 4-1BB, OX40, CD30, CD40, PD-1, ICOS, lymphocyte function-associated antigen-1 (LFA-1), CD2, CD7, LTGHT, NKG2C, B7-H3, a ligand that specifically binds with CD83.

A “co-stimulatory molecule” refers to the cognate binding partner on a T-cell that specifically binds with a co-stimulatory ligand, thereby mediating a co-stimulatory response by the cell, such as, but not limited to proliferation. Co-stimulatory molecules include, but are not limited to, an MHC class I molecule, BTLA and Toll ligand receptor. Examples of costimulatory molecules include CD27, CD28, CD8, 4-1BB (CD137), OX40, CD30, CD40, PD-1, ICOS, lymphocyte function-associated antigen-1 (LFA-1), CD2, CD7, LIGHT, NKG2C, B7-H3 and a ligand that specifically binds with CD83 and the like.

In a preferred embodiment, the signal transduction domain of the CAR of the present invention comprises a part of co-stimulatory signal molecule selected from the group consisting of fragment of 4-1BB (GenBank: AAA53133.) and CD28 (NP_006130.1). In particular the signal transduction domain of the CAR of the present invention comprises amino acid sequence which comprises at least 70%, preferably at least 80%, more preferably at least 90%, 95% 97% or 99% sequence identity with amino acid sequence selected from the group consisting of SEQ ID NO: 8.

A CAR according to the present invention is expressed on the surface membrane of the cell. Thus, such CAR further comprises a transmembrane domain. The distinguishing features of appropriate transmembrane domains comprise the ability to be expressed at the surface of a cell, preferably in the present invention an immune cell, in particular lymphocyte cells or Natural killer (NK) cells, and to interact together for directing cellular response of immune cell against a predefined target cell. The transmembrane domain can be derived either from a natural or from a synthetic source. The transmembrane domain can be derived from any membrane-bound or transmembrane protein. As non-limiting examples, the transmembrane polypeptide can be a subunit of the T-cell receptor such as α, β, γ or ζ, polypeptide constituting CD3 complex, IL2 receptor p55 (α chain), p75 (β chain) or γ chain, subunit chain of Fc receptors, in particular Fcγ receptor III or CD proteins. Alternatively the transmembrane domain can be synthetic and can comprise predominantly hydrophobic residues such as leucine and valine. In a preferred embodiment said transmembrane domain is derived from the human CD8 alpha chain (e.g. NP_001139345.1) The transmembrane domain can further comprise a hinge region between said extracellular ligand-binding domain and said transmembrane domain. The term “hinge region” used herein generally means any oligo- or polypeptide that functions to link the transmembrane domain to the extracellular ligand-binding domain. In particular, hinge region are used to provide more flexibility and accessibility for the extracellular ligand-binding domain. A hinge region may comprise up to 300 amino acids, preferably 10 to 100 amino acids and most preferably 25 to 50 amino acids. Hinge region may be derived from all or part of naturally occurring molecules, such as from all or part of the extracellular region of CD8, CD4 or CD28, or from all or part of an antibody constant region. Alternatively the hinge region may be a synthetic sequence that corresponds to a naturally occurring hinge sequence, or may be an entirely synthetic hinge sequence. In a preferred embodiment said hinge domain comprises a part of FcγRIII receptor, human CD8 alpha chain or IgG1 respectively referred to in this specification as SEQ ID NO. 3, SEQ ID NO. 4 and SEQ ID NO.5, or hinge polypeptides which display preferably at least 80%, more preferably at least 90%, 95% 97% or 99% sequence identity with these polypeptides.

A car according to the invention generally further comprises a transmembrane domain (TM) more particularly selected from CD8a and 4-1BB, showing identity with the polypeptides of SEQ ID NO. 6 or 7.

TABLE 1
Sequence of the different CAR components

| Functional domains | SEQ ID # | Raw amino acid sequence |
|---|---|---|
| CD8α signal peptide | SEQ ID NO. 1 | MALPVTALLLPLALLLHAARP |
| Alternative signal peptide | SEQ ID NO. 2 | METDTLLLWVLLLWVPGSTG |
| FcγRIIIα hinge | SEQ ID NO. 3 | GLAVSTISSFFPPGYQ |
| CD8α hinge | SEQ ID NO. 4 | TTTPAPRPPTPAPTIASQPLSLRPEAC |
| | | RPAAGGAVHTRGLDFACD |
| IgG1 hinge | SEQ ID NO. 5 | EPKSPDKTHTCPPCPAPPVAGPSVFLF |
| | | PPKPKDTLMIARTPEVTCVVVDVSHED |
| | | PEVKFNWYVDGVEVHNAKTKPREEQYN |
| | | STYRVVSVLTVLHQDWLNGKEYKCKVS |
| | | NKALPAPIEKTISKAKGQPREPQVYTL |
| | | PPSRDELTKNQVSLTCLVKGFYPSDIA |
| | | VEWESNGQPENNYKTTPPVLDSDGSFF |
| | | LYSKLTVDKSRWQQGNVFSCSVMHEAL |
| | | HNHYTQKSLSLSPGK |
| CD8α transmembrane domain | SEQ ID NO. 6 | IYIWAPLAGTCGVLLLSLVITLYC |
| 41BB transmembrane domain | SEQ ID NO. 7 | IISFFLALTSTALLFLLFFLTLRFSVV |
| 41BB intracellular domain | SEQ ID NO. 8 | KRGRKKLLYIFKQPFMRPVQTTQEEDG |
| | | CSCRFPEEEEGGCEL |
| CD3ζ intracellular domain | SEQ ID NO. 9 | RVKFSRSADAPAYQQGQNQLYNELNLG |
| | | RREEYDVLDKRRGRDPEMGGKPRRKNP |
| | | QEGLYNELQKDKMAEAYSEIGMKGERR |
| | | RGKGHDGLYQGLSTATKDTYDALHMQA |
| | | LPPR |
| G4Sx3 linker | SEQ ID NO. 10 | GGGGSGGGGSGGGGS |

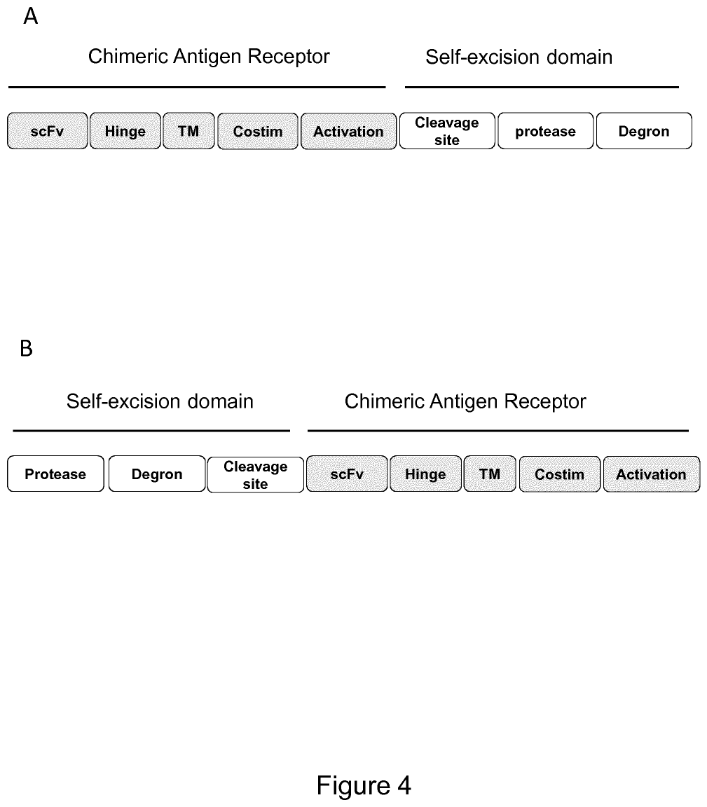

A chimeric antigen receptor according to the present invention may be a single chain CAR, meaning that all domains of said CAR are included into one polypeptide chain or a multi-chain CAR. Multi-chain CARs are chimeric antigen receptors formed of multiple polypeptides, so that typically at least one ectodomain and the at least one endodomain are born on different polypeptide chains. The different polypeptide chains are anchored into the membrane in a close proximity allowing interactions with each other. In such architectures, the signaling and co-stimulatory domains can be in juxtamembrane positions (i.e. adjacent to the cell membrane on the internal side of it), which is deemed to allow improved function of co-stimulatory domains. The multi-subunit architecture is deemed offering more flexibility and capabilities of designing CARs with more control on T-cell activation. For instance, it is possible to include several extracellular antigen recognition domains having different specificity to obtain a multi-specific CAR architecture. It is also possible to control the relative ratio between the different subunits into the multi-chain CAR. This type of architecture has been described by the applicant in WO2014039523, in particular in FIG. 4, which is incorporated by reference.

Accordingly, a multi-chain CAR according to the invention may be one of which comprises at least one ectodomain comprising:
i) an extracellular antigen binding domain; and
ii) one transmembrane domain; and
   and at least one endodomain comprising a signal transducing domain, and optionally a co-stimulatory domain;

According to certain embodiments, a multi-chain CAR of the invention may further comprise a third polypeptide chain comprising:
i) at least one endodomain comprising a co-stimulatory domain; and
ii) at least one transmembrane domain.

The different chains as part of a single multi-chain CAR can be assembled, for instance, by using the different alpha, beta and gamma chains of the high affinity receptor for IgE (FcεRI), for instance by replacing the high affinity IgE binding domain of the FcεRI alpha chain by an ectodomain, whereas the N and/or C-termini tails of FcεRI beta and/or gamma chains are fused to an endodomain comprising a signal transducing domain and co-stimulatory domain, respectively. The extracellular ligand binding domain has the role of redirecting T-cell specificity towards cell targets, while the signal transducing domains activate the immune cell response. The fact that the different polypeptide chains derived from the alpha, beta and gamma polypeptides from FcεRI are transmembrane polypeptides sitting in juxtamembrane position, provides a more flexible architecture to CARs, improving specificity towards the antigen target and reducing background activation of immune cells.

According to the present invention, at least one component (e.g. polypeptide) of a multi-chain CAR as previously described can be coupled to a degron and/or protease domain to form a chimeric polynucleotide or polypeptide as described herein, in view of expressing a conditional multi-chain CAR.

The genetic sequences encoding CARs are generally introduced into the cells genome using retroviral vectors, especially lentiviral vectors as reviewed by Liechtenstein, T., et al. [Lentiviral Vectors for Cancer Immunotherapy and Clinical Applications (2013) Cancers. 5(3):815-837]. Lentiviral vectors have elevated transduction efficiency but integrate at random locations. As an alternative, the chimeric polynucleotides encoding the components of chimeric antigen receptor (CAR) according to the present invention can be introduced at selected loci by site-directed gene insertion by homologous recombination or NHEJ using rare-cutting endonucleases as described in U.S. Pat. No. 8,921,332.

According to a preferred embodiment of the invention, the chimeric polynucleotides encoding the CAR components of the present invention are inserted at the TCR locus as suggested by Macleod D., et al. [Integration of a CD19 CAR into the TCR Alpha Chain Locus Streamlines Production of Allogeneic Gene-Edited CAR T Cells (2017) Molecular Therapy 25(4):949-961] or even preferably at other loci which transcriptional activity is under control of endogenous promoters which are up-regulated by immune cell activation.

Also the invention more particularly relates to chimeric polypeptides according to the present invention that generally comprise a first polypeptide coding for a CAR and second polypeptide comprising a protease or a degron domain. In general, said first polypeptide codes for a single-chain CAR or a transmembrane subunit of a multi-chain CAR, wherein said first polypeptide preferably comprises:
a transmembrane domain linked to an extra cellular ligand binding-domain comprising VH and VL from a monoclonal antibody.
a transmembrane from CD8a transmembrane domain.
a cytoplasmic domain including a CD3 zeta signaling domain
and optionally a co-stimulatory domain from CD28 or 4-1BB.

According to some embodiments, said first polypeptide may further comprise a hinge such as a CD8a hinge, IgG1 hinge or FcγRIIIα hinge.

The CARs according to the present invention preferably targets an antigen selected from CD19, CD22, CD33, CD38, CD123, CS1, CLL1, ROR1, OGD2, BCMA, HSP70 and EGFRvIII.

The effector immune cells expressing the chimeric polynucleotides according to the present invention are preferably primary immune cells, such as NK or T-cells.

By “immune cell” is meant a cell of hematopoietic origin functionally involved in the initiation and/or execution of innate and/or adaptative immune response, such as typically CD3 or CD4 positive cells. The immune cell according to the present invention can be a dendritic cell, killer dendritic cell, a mast cell, a NK-cell, a B-cell or a T-cell selected from the group consisting of inflammatory T-lymphocytes, cytotoxic T-lymphocytes, regulatory T-lymphocytes or helper T-lymphocytes. Cells can be obtained from a number of non-limiting sources, including peripheral blood mononuclear cells, bone marrow, lymph node tissue, cord blood, thymus tissue, tissue from a site of infection, ascites, pleural effusion, spleen tissue, and from tumors, such as tumor infiltrating lymphocytes. In some embodiments, said immune cell can be derived from a healthy donor, from a patient diagnosed with cancer or from a patient diagnosed with an infection. In another embodiment, said cell is part of a mixed population of immune cells which present different phenotypic characteristics, such as comprising CD4, CD8 and CD56 positive cells.

By “primary cell” or “primary cells” are intended cells taken directly from living tissue (e.g. biopsy material) and established for growth in vitro for a limited amount of time, meaning that they can undergo a limited number of population doublings. Primary cells are opposed to continuous tumorigenic or artificially immortalized cell lines. Non-limiting examples of such cell lines are CHO-K1 cells; HEK293 cells; Caco2 cells; U2-OS cells; NIH 3T3 cells; NSO cells; SP2 cells; CHO-S cells; DG44 cells; K-562 cells, U-937 cells; MRC5 cells; IMR90 cells; Jurkat cells; HepG2 cells; HeLa cells; HT-1080 cells; HCT-116 cells; Hu-h7 cells; Huvec cells; Molt 4 cells. Primary cells are generally used in cell therapy as they are deemed more functional and less tumorigenic.

In general, primary immune cells are provided from donors or patients through a variety of methods known in the art, as for instance by leukapheresis techniques as reviewed by Schwartz J. et al. (Guidelines on the use of therapeutic apheresis in clinical practice-evidence-based approach from the Writing Committee of the American Society for Apheresis: the sixth special issue (2013) J Clin Apher. 28(3):145-284).

The primary immune cells according to the present invention can also be differentiated from stem cells, such as cord blood stem cells, progenitor cells, bone marrow stem cells, hematopoietic stem cells (HSC) and induced pluripotent stem cells (iPS).

The transformation of an immune cell with a chimeric polynucleotide of the present invention results into an “engineered immune cell” in the sense of the present invention. Such transformation can be made by the various methods known in the art such as viral vector transduction or RNA transfection.

According to one embodiment, the chimeric polypeptide according to the invention comprises a first polypeptide encoding a chimeric antigen receptor and a second polypeptide comprising a protease having cleavage activity directed against the first polypeptide.

In general, the protease is a specific protease, which is active against a particular polypeptide motif or sequence referred to herein as “cleavage domain”. According to such embodiment, this cleavage domain can be comprised within the first polypeptide that codes for the chimeric antigen receptor, so that when the protease is expressed, the CAR is cleaved and becomes inactive. According to an alternative embodiment, the cleavage domain can be set outside the CAR, preferably into the polypeptide sequence linking the first and second polypeptide, so that the second polypeptide is excised from the first. In such a configuration, the protease can mature a functional CAR, which can be released from the initial chimeric polypeptide and then presented at the surface of the cell in order to become active by binding a specific antigen. Thereby, said protease, depending on the architecture of the chimeric polypeptide, can respectively have the effect of preventing presentation of the CAR polypeptide at the surface of the transformed immune cell, or converting an inactive CAR precursor into a functional CAR.

According to one embodiment of the present invention, said protease activity can be inhibited by a protease inhibitor that will act alternatively as a switch-on or a switch-off molecule. Referring to the previous embodiments, wherein the protease prevents the presentation of a functional CAR at the cell surface, the adjunction of protease inhibitor will result into proper presentation of the CAR at the surface and its possible interaction with a specific antigen, thereby acting as a switch on with respect to the engineered immune cell. By contrast, if the protease processes an active CAR, the adjunction of the protease inhibitor will prevent the presentation of functional CARs and act as a switch-off with respect to the activation of the engineered immune cells.

Different protease and protease inhibitors can be used in the present invention, in particular small molecules approved for antiviral therapy, such as antiretroviral HIV-1 protease inhibitors or hepatitis C virus NS3/4A protease inhibitors. Examples of antiretroviral HIV-1 protease inhibitors are amprenavis, atazanavir, darunavir, fosamprenavir, indinavir, lopinavir, nelfinavir, ritonavir, saquinavir or tipanavir. Preferred hepatitis C virus NS3/4A protease inhibitors are asunaprevir, boceprevir, grazoprevir, paritaprevir, simeprevir and telaprevir. Most preferred is asunaprevir to inhibit protease activity of proteases that share identity with the nonstructural protein 3 (NS3) protease.

TABLE 2
Examples of protease and protease inhibitors

| Protease | Protease inhibitors |
|---|---|
| NS3/4A protease | |
| IUBMB Enzyme Nomenclature | |
| EC 3.4.21.98 | |
| | asunaprevir |
| | boceprevir |
| | grazoprevir |
| | paritaprevir |
| | simeprevir |
| | telaprevir |
| HIV-1 protease | |
| IUBMB Enzyme Nomenclature | |
| EC 3.4.23.16 | |
| | amprenavis |
| | atazanavir |
| | darunavir |
| | fosamprenavir |
| | indinavir |
| | lopinavir |
| | nelfinavir |
| | ritonavir |
| | saquinavir |
| | tipanavir |

According to one embodiment, the chimeric polypeptide according to the invention further comprises at least one degron polypeptide sequence.

By “degron” is meant any polypeptide sequence identified in the literature as functional elements that are used by E3 ubiquitin ligases to target proteins for degradation. Most degrons are short linear motifs embedded within the sequences of modular proteins. Degrons are typically composed of 5 to 20, preferably 6 to 10 amino acids and are generally located within flexible regions of proteins so that the degrons can easily interact with other proteins. Degrons enable the elimination of proteins that are no longer required, preventing their possible dysfunction.

A well-characterized example of an E3 ligase-degron pair is the degron in p53 and the E3 ligase MDM2 (murine double minute 2), which is a RING domain-containing individual E3 ligase (49). In the absence of DNA damage or other stress signals, MDM2 targets the constantly produced p53 for degradation. The structure formed between MDM2 and p53 shows that a short segment on the N-terminal region of p53, corresponding to the degron motif, forms an a-helical stretch that binds to the SWIB domain of MDM2 [Kussie, S. et al. (1996) Structure of the MDM2 oncoprotein bound to the p53 tumor suppressor transactivation domain. Science. 274, 948-953].

Degrons are classified as ubiquitin-dependent or ubiquitin-independent, proteasomal or lysosomal. The one used in the present invention is preferably bifonctional, meaning that it is both proteasomal and lysosomal, such as that used in the examples comprising the polypeptide SEQ ID NO. 32, 38, 41 or 43.

Such degron polypeptides can be introduced into the chimeric polypeptide to enhance intracellular degradation of CAR, thereby preventing presentation of the CAR at the cell surface. According to a preferred embodiment, the degron is comprised into the second polypeptide comprised into the chimeric polypeptide of the present invention, which is preferably excised by the protease.

Examples of chimeric polypeptides architectures according to the present invention are illustrated in the following Tables 3 to 8.

TABLE 3
Chimeric polypeptide of structure V-1

| Chimeric | | | | | | | | | | |
|---|---|---|---|---|---|---|---|---|---|---|
| polypeptide | | | | | | | | | | |
| designation | Structure | | | | | | | | | |
| V-1A-OFF | signal | VH | VL | FcyRIIIα | CD8α | 41BB-IC | CD3zeta | Cleavage | protease | degron |
| | peptide | | | hinge | TM | | | domain | | |
| | (optional) | | | | | | | | | |
| V-1B-ON | signal | VH | VL | FcyRIIIα | CD8α | 41BB-IC | CD3zeta | Cleavage | protease | |
| | peptide | | | hinge | TM | | | domain | | |
| | (optional) | | | | | | | | | |
| V1-C-OFF | signal | VH | VL | FcyRIIIα | CD8α | 41BB-IC | CD3zeta | Cleavage | degron | protease |
| | peptide | | | hinge | TM | | | domain | | |
| | (optional) | | | | | | | | | |
| V1-D-OFF | signal | VH | VL | FcyRIIIα | Cleavage | CD8α | 41BB-IC | CD3zeta | 2A | protease |
| | peptide | | | hinge | domain | TM | | | | |
| | (optional) | | | | | | | | | |

TABLE 4
Chimeric polypeptide of structure V-2

| CAR | | | | | | | | | | |
|---|---|---|---|---|---|---|---|---|---|---|
| Designation | CAR Structure | | | | | | | | | |
| V-2A-OFF | signal | VH | VL | FcyRIIIα | 41BB-TM | 41BB-IC | CD3zeta | Cleavage | protease | degron |
| | peptide | | | hinge | | | | domain | | |
| | (optional) | | | | | | | | | |
| V-2B-ON | signal | VH | VL | FcyRIIIα | 41BB-TM | 41BB-IC | CD3zeta | Cleavage | protease | |
| | peptide | | | hinge | | | | domain | | |
| | (optional) | | | | | | | | | |
| V-2C-OFF | signal | VH | VL | FcyRIIIα | 41BB-TM | 41BB-IC | CD3zeta | Cleavage | degron | protease |
| | peptide | | | hinge | | | | domain | | |
| | (optional) | | | | | | | | | |
| V-2D-OFF | signal | VH | VL | FcyRIIIα | Cleavage | 41BB-TM | 41BB-IC | CD3zeta | 2A | protease |
| | peptide | | | hinge | domain | | | | | |
| | (optional) | | | | | | | | | |

TABLE 5
Chimeric polypeptide of structure V-3

| CAR | | | | | | | | | | |
|---|---|---|---|---|---|---|---|---|---|---|
| Designation | CAR Structure | | | | | | | | | |
| V-3A-OFF | signal | VH | VL | CD8α | CD8α | 41BB-IC | CD3zeta | Cleavage | protease | degron |
| | peptide | | | hinge | TM | | | domain | | |
| | (optional) | | | | | | | | | |
| V-3B-ON | signal | VH | VL | CD8α | CD8α | 41BB-IC | CD3zeta | Cleavage | protease | |
| | peptide | | | hinge | TM | | | domain | | |
| | (optional) | | | | | | | | | |
| V-3C-OFF | signal | VH | VL | CD8α | CD8α | 41BB-IC | CD3zeta | Cleavage | degron | protease |
| | peptide | | | hinge | TM | | | domain | | |
| | (optional) | | | | | | | | | |
| V-3D-OFF | signal | VH | VL | CD8α | Cleavage | CD8α | 41BB-IC | CD3zeta | 2A | protease |
| | peptide | | | hinge | domain | TM | | | | |
| | (optional) | | | | | | | | | |

TABLE 6
Chimeric polypeptide of structure V-4

| CAR | | | | | | | | | | |
|---|---|---|---|---|---|---|---|---|---|---|
| Designation | CAR Structure | | | | | | | | | |
| V-4A-OFF | signal | VH | VL | CD8α | 41BB-TM | 41BB-IC | CD3zeta | Cleavage | protease | degron |
| | peptide | | | hinge | | | | domain | | |
| | (optional) | | | | | | | | | |
| V-4B-ON | signal | VH | VL | CD8α | 41BB-TM | 41BB-IC | CD3zeta | Cleavage | protease | |
| | peptide | | | hinge | | | | domain | | |
| | (optional) | | | | | | | | | |
| V-4C-OFF | signal | VH | VL | CD8α | 41BB-TM | 41BB-IC | CD3zeta | Cleavage | degron | protease |
| | peptide | | | hinge | | | | domain | | |
| | (optional) | | | | | | | | | |
| V-4D-OFF | signal | VH | VL | CD8α | Cleavage | 41BB-TM | 41BB-IC | CD3zeta | 2A | protease |
| | peptide | | | hinge | domain | | | | | |
| | (optional) | | | | | | | | | |

TABLE 7
Chimeric polypeptide of structure V-5

| CAR | | | | | | | | | | |
|---|---|---|---|---|---|---|---|---|---|---|
| Designation | CAR Structure | | | | | | | | | |
| V-5A-OFF | signal | VH | VL | IgG1 | CD8α | 41BB-IC | CD3zeta | Cleavage | protease | degron |
| | peptide | | | hinge | TM | | | domain | | |
| | (optional) | | | | | | | | | |
| V-5B-ON | signal | VH | VL | IgG1 | CD8α | 41BB-IC | CD3zeta | Cleavage | protease | |
| | peptide | | | hinge | TM | | | domain | | |
| | (optional) | | | | | | | | | |
| V-5C-OFF | signal | VH | VL | IgG1 | CD8α | 41BB-IC | CD3zeta | Cleavage | degron | protease |
| | peptide | | | hinge | TM | | | domain | | |
| | (optional) | | | | | | | | | |
| V-5D-OFF | signal | VH | VL | IgG1 | Cleavage | CD8α | 41BB-IC | CD3zeta | 2A | protease |
| | peptide | | | hinge | domain | TM | | | | |
| | (optional) | | | | | | | | | |

TABLE 8
Chimeric polypeptide of structure V-6

| CAR | | | | | | | | | | |
|---|---|---|---|---|---|---|---|---|---|---|
| Designation | CAR Structure | | | | | | | | | |
| V-6A-OFF | signal | VH | VL | IgG1 | 41BB-TM | 41BB-IC | CD3zeta | Cleavage | protease | degron |
| | peptide | | | hinge | | | | domain | | |
| | (optional) | | | | | | | | | |
| V-6B-ON | signal | VH | VL | IgG1 | 41BB-TM | 41BB-IC | CD3zeta | Cleavage | protease | |
| | peptide | | | hinge | | | | domain | | |
| | (optional) | | | | | | | | | |
| V-6C-OFF | signal | VH | VL | IgG1 | 41BB-TM | 41BB-IC | CD3zeta | Cleavage | degron | protease |
| | peptide | | | hinge | | | | domain | | |
| | (optional) | | | | | | | | | |
| V-6D-OFF | signal | V H | VL | IgG1 | Cleavage | 41BB-TM | 41BB-IC | CD3zeta | 2A | protease |
| | peptide | | | hinge | domain | | | | | |
| | (optional) | | | | | | | | | |

According to one aspect of the invention, the extracellular binding domain of the CAR or recombinant T-cell receptor can include particular epitopes which can be recognized by specific antibodies, preferably therapeutically approved antibodies, such as those listed in Table 9.

TABLE 9
Examples of mAb-specific epitopes also called
mimotope (and their corresponding mAbs) that
may be included in the extracellular domain
of the CAR of the invention.
Rituximab

| | | |
|---|---|---|
| Mimotope | SEQ ID NO 11 | CPYSNPSLC |

Palivizumab

| | | |
|---|---|---|
| Epitope | SEQ ID NO 12 | NSELLSLINDMPITNDQKKLMSNN |

Cetuximab

| | | |
|---|---|---|
| Mimotope 1 | SEQ ID NO 13 | CQFDLSTRRLKC |
| Mimotope 2 | SEQ ID NO 14 | CQYNLSSRALKC |
| Mimotope 3 | SEQ ID NO 15 | CVWQRWQKSYVC |
| Mimotope 4 | SEQ ID NO 16 | CMWDRFSRWYKC |

Nivolumab

| | | |
|---|---|---|
| Epitope 1 | SEQ ID NO 17 | SFVLNWYRMSPSNQTDKLAAFPED |
| | | R |
| Epitope 2 | SEQ ID NO 18 | SGTYLCGAISLAPKAQIKE |

QBEND-10

| | | |
|---|---|---|
| Epitope | SEQ ID NO 19 | ELPTQGTFSNVSTNVSPAKPTTTA |

Alemtuzumab

| | | |
|---|---|---|
| Epitope | SEQ ID NO 20 | GQNDTSQTSSPS |

Accordingly, a chimeric polypeptide according to the invention can comprise a polypeptide sequence of an extracellular binding domain comprising one of the following sequence:
V₁-L₁-V₂-(L)ₓ-Epitope1-(L)ₓ-;
V₁-L₁-V₂-(L)-Epitope1-(L)-Epitope2-(L)_{X}-;
V₁-L₁-V₂-(L)ₓ-Epitope1-(L)ₓ-Epitope2-(L)ₓ-Epitope3-(L)ₓ-;
(L)ₓ-Epitope1-(L)ₓ-V₁-L₁-V₂;
(L)ₓ-Epitope1-(L)ₓ-Epitope2-(L)ₓ-V₁-L₁-V₂;
Epitope1-(L)ₓ-Epitope2-(L)ₓ-Epitope3-(L)ₓ-V₁-L₁-V₂;
(L)ₓ-Epitope1-(L)ₓ-V₁-L₁-V₂-(L)ₓ-Epitope2-(L)ₓ;
(L)ₓ-Epitope1-(L)ₓ-V₁-L₁-V₂-(L)ₓ-Epitope2-(L)ₓ-Epitope3-(L)ₓ-;
(L)ₓ-Epitope1-(L)ₓ-V₁-L₁-V₂-(L)ₓ-Epitope2-(L)ₓ-Epitope3-(L)ₓ-Epitope4-(L)ₓ-;
(L)ₓ-Epitope1-(L)ₓ-Epitope2-(L)ₓ-V₁-L₁-V₂-(L)ₓ-Epitope3-(L)ₓ-;
(L)ₓ-Epitope1-(L)ₓ-Epitope2-(L)ₓ-V₁-L₁-V₂-(L)ₓ-Epitope3-(L)ₓ-Epitope4-(L)ₓ-;
V₁-(L)ₓ-Epitope1-(L)ₓ-V₂;
V₁-(L)ₓ-Epitope1-(L)ₓ-V₂-(L)ₓ-Epitope2-(L)ₓ;
V₁-(L)ₓ-Epitope1-(L)ₓ-V₂-(L)ₓ-Epitope2-(L)ₓ-Epitope3-(L)ₓ;
V₁-(L)ₓ-Epitope1-(L)ₓ-V₂-(L)ₓ-Epitope2-(L)ₓ-Epitope3-(L)ₓ-Epitope4-(L)ₓ;
(L)ₓ-Epitope1-(L)ₓ-V₁-(L)ₓ-Epitope2-(L)ₓ-V₂; or,
(L)ₓ-Epitope1-(L)ₓ-V,-(L)ₓ-Epitope2-(L)ₓ-V₂-(L)ₓ-Epitope3-(L)ₓ;
wherein,
V₁ is V_{L} and V₂ is V_{H} or V₁ is V_{H} and V₂ is V_{L};
L₁ is a linker suitable to link the V_{H} chain to the V_{L} chain;
L is a linker comprising glycine and serine residues, and each occurrence of L in the extracellular binding domain can be identical or different to other occurrence of L in the same extracellular binding domain, and,
x is 0 or 1 and each occurrence of x is selected independently from the others; and,
Epitope 1, Epitope 2 and Epitope 3 are mAb-specific epitopes, such as those in Table 3, and can be identical or different.

Still according to the invention, L₁ can be a linker comprising Glycine and/or Serine and can comprise the amino acid sequence (Gly-Gly-Gly-Ser)ₙ or (Gly-Gly-Gly-Gly-Ser)ₙ, where n is 1, 2, 3, 4 or 5 or a linker comprising the amino acid sequence (Gly₄Ser)₄ or (Gly₄Ser)₃.

L can be a linker comprising Glycine and/or Serine having an amino acid sequence selected from SGG, GGS, SGGS, SSGGS, GGGG, SGGGG, GGGGS, SGGGGS, GGGGGS, SGGGGGS, SGGGGG, GSGGGGS, GGGGGGGS, SGGGGGGG, SGGGGGGGS, or SGGGGSGGGGS.

Epitope 1, Epitope 2, Epitope 3 and Epitope 4 can be independently selected from mAb-specific epitopes specifically recognized by ibritumomab, tiuxetan, muromonab-CD3, tositumomab, abciximab, basiliximab, brentuximab vedotin, cetuximab, infliximab, rituximab, alemtuzumab, bevacizumab, certolizumab pegol, daclizumab, eculizumab, efalizumab, gemtuzumab, natalizumab, omalizumab, palivizumab, ranibizumab, tocilizumab, trastuzumab, vedolizumab, adalimumab, belimumab, canakinumab, denosumab, golimumab, ipilimumab, ofatumumab, panitumumab, QBEND-10 and ustekinumab. In a preferred embodiment said Epitope 1, Epitope 2, are specifically recognized by rituximab and epitope 3 is specifically recognized by QBEND-10.

The present invention encompasses the polynucleotide sequences encoding a chimeric polypeptide described herein and any vectors comprising such polynucleotides according to the present invention. According to one aspect of the present invention, the first polypeptide encoding a chimeric antigen receptor (CAR) and the second polypeptide encoding a protease, are encoded by separate polynucleotides or vectors, referred to as a set of polynucleotides, which can be co-transfected or co-expressed in the cells.

Preferred CARs according to the present invention are those with polynucleotide and polypeptide sequences displaying identity with those detailed in the examples, especially a CAR anti-CD22 sharing identity with SEQ ID NO:68 or a polynucleotide sequence comprising a sequence sharing identity with SEQ ID NO:63. It is also provided, as a preferred embodiment illustrated in Example 8, a polynucleotide sharing identity with SEQ ID NO:59 to be used as an insertion matrix for insertion of a CAR according to the present invention at the TCR locus, especially an AAV vector or lentiviral vector comprising same.

The present invention further relates to the engineered immune cells transformed with a polynucleotide encoding a chimeric polypeptide as per the present invention that typically comprises an effector polypeptide, a protease domain, and a degron. Such immune cells are preferably primary cells, such as a T-cell or a NK cell. Still according to the invention, immune cells, in which the expression of TCR is reduced or suppressed are preferred for their allogeneic use in cell therapy treatments. In some embodiments, the expression of at least one MHC protein, preferably β2m or HLA, can also be reduced or suppressed to increase their persistence in-vivo.

The present invention broadly provides with a method for inactivating (switching-off) a function linked to a transmembrane receptor into an effector cell, comprising at least one of the following steps:
providing an effector cell,
introducing into an effector cell a polynucleotide, or set of polynucleotide sequences according to the invention, encoding more particularly a chimeric polypeptide comprising a receptor polypeptide, a protease, and a degron;
expressing said chimeric polypeptide into said cell so that the protease activity removes the degron and said receptor polypeptide is presented at the surface of the cell;
introducing a protease inhibitor into the cell's environment, which inhibits said protease activity; such that the degron is not removed anymore and said expressed chimeric polypeptide is degraded by the proteasome, thereby switching off the function linked to the transmembrane receptor in said effector cell.

The present invention also provides with a method for activating (switching-on) a function linked to a transmembrane receptor into an effector cell, comprising at least the following steps:
providing an effector cell,
introducing into said effector cell a set of polynucleotide sequences or a unique polynucleotide encoding (i) a transmembrane receptor polypeptide and (ii) a protease domain that is directed against said transmembrane receptor polypeptide,
expressing into said effector cell said polypeptides, the protease activity of which inactivates said receptor polypeptide function,
introducing a protease inhibitor in the immune cell's environment, in order to inhibit said protease activity and allow the transmembrane receptor to be presented at the cell surface, thereby activating the function of said receptor into said effector cell.

As previously stated, the transmembrane receptor can be for instance a CAR or a recombinant TCR, or any transmembrane receptor polypeptide that binds a surface marker of a pathological cell.

According to one embodiment, said polynucleotide sequences encoding (i) a transmembrane receptor polypeptide and (ii) a protease domain that is directed against said transmembrane receptor polypeptide can be encoded by a single polynucleotide separated by IRES (Internal Ribosome Entry Site) or a 2A peptide.

The above methods are preferably used for the treatment of a disease, wherein said effector immune cells endowed with the transmembrane receptor polypeptide contribute to eliminate pathological cells, such as malignant or infected cells in a patient.

Engineered Immune Cells and Populations of Immune Cells

The present invention is also drawn to the variety of engineered immune cells obtainable according to one of the method described previously under isolated form or as part of populations of cells. In particular, the present invention is directed to cells comprising any of the polypeptide or polynucleotide sequences referred to in the present invention, especially cells expressing a CAR as described herein.

According to a preferred aspect of the invention the engineered cells are primary immune cells, such as NK cells or T-cells, which are generally part of populations of cells that may involve different types of cells. In general, population deriving from patients or donors isolated by leukapheresis from PBMC (peripheral blood mononuclear cells).

According to a preferred aspect of the invention, more than 50% of the immune cells comprised in said population are TCR negative T-cells. According to a more preferred aspect of the invention, more than 50% of the immune cells comprised in said population are CAR positive T-cells.

The present invention encompasses immune cells comprising any combinations of the different exogenous coding sequences and gene inactivation, which have been respectively and independently described above. Among these combinations are particularly preferred those combining the expression of a CAR under the transcriptional control of an endogenous promoter that is steadily active during immune cell activation and preferably independently from said activation, and the expression of an exogenous sequence encoding a cytokine, such as IL-2, IL-12 or IL-15, under the transcriptional control of a promoter that is up-regulated during the immune cell activation.

Another preferred combination is the insertion of an exogenous sequence encoding a CAR or one of its constituents under the transcription control of the hypoxia-inducible factor 1 gene promoter (Uniprot: Q16665).

The invention is also drawn to a pharmaceutical composition comprising an engineered primary immune cell or immune cell population as previously described for the treatment of infection or cancer, and to a method for treating a patient in need thereof, wherein said method comprises:
preparing a population of engineered primary immune cells according to the method of the invention as previously described;
optionally, purifying or sorting said engineered primary immune cells;
activating said population of engineered primary immune cells upon or after infusion of said cells into said patient.

Activation and Expansion of T Cells

Whether prior to or after genetic modification, the immune cells according to the present invention can be activated or expanded, even if they can activate or proliferate independently of antigen binding mechanisms. T-cells, in particular, can be activated and expanded using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005. T cells can be expanded in vitro or in vivo. T cells are generally expanded by contact with an agent that stimulates a CD3 TCR complex and a co-stimulatory molecule on the surface of the T cells to create an activation signal for the T-cell. For example, chemicals such as calcium ionophore A23187, phorbol 12-myristate 13-acetate (PMA), or mitogenic lectins like phytohemagglutinin (PHA) can be used to create an activation signal for the T-cell.

As non-limiting examples, T cell populations may be stimulated in vitro such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. Conditions appropriate for T cell culture include an appropriate media (e.g., Minimal Essential Media or RPMI Media 1640 or, X-vivo 5, (Lonza)) that may contain factors necessary for proliferation and viability, including serum (e.g., fetal bovine or human serum), interleukin-2 (IL-2), insulin, IFN-g, 1L-4, 1L-7, GM-CSF, -10, -2, 1L-15, TGFp, and TNF- or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, A1M-V, DMEM, MEM, a-MEM, F-12, X-Vivo 1, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% C02). T cells that have been exposed to varied stimulation times may exhibit different characteristics

In another particular embodiment, said cells can be expanded by co-culturing with tissue or cells. Said cells can also be expanded in vivo, for example in the subject's blood after administrating said cell into the subject.

Therapeutic Compositions and Applications

The method of the present invention described above allows producing engineered primary immune cells within a limited time frame of about 15 to 30 days, preferably between 15 and 20 days, and most preferably between 18 and 20 days so that they keep their full immune therapeutic potential, especially with respect to their cytotoxic activity.

These cells form a population of cells, which preferably originate from a single donor or patient. These populations of cells can be expanded under closed culture recipients to comply with highest manufacturing practices requirements and can be frozen prior to infusion into a patient, thereby providing “off the shelf” or “ready to use” therapeutic compositions.

As per the present invention, a significant number of cells originating from the same Leukapheresis can be obtained, which is critical to obtain sufficient doses for treating a patient. Although variations between populations of cells originating from various donors may be observed, the number of immune cells procured by a leukapheresis is generally about from 10⁸ to 10¹⁰ cells of PBMC. PBMC comprises several types of cells: granulocytes, monocytes and lymphocytes, among which from 30 to 60% of T-cells, which generally represents between 108 to 10⁹ of primary T-cells from one donor. The method of the present invention generally ends up with a population of engineered cells that reaches generally more than about 10⁸ T-cells, more generally more than about 10⁹ T-cells, even more generally more than about 10¹⁰ T-cells, and usually more than 10¹¹ T-cells.

The invention is thus more particularly drawn to a therapeutically effective population of primary immune cells, wherein at least 30%, preferably 50%, more preferably 80% of the cells in said population have been modified according to any one the methods described herein. Said therapeutically effective population of primary immune cells, as per the present invention, comprises immune cells that have integrated at least one exogenous genetic sequence under the transcriptional control of an endogenous promoter from at least one of the genes listed in Table 5.

Such compositions or populations of cells can therefore be used as medicaments; especially for treating cancer, particularly for the treatment of lymphoma, but also for solid tumors such as melanomas, neuroblastomas, gliomas or carcinomas such as lung, breast, colon, prostate or ovary tumors in a patient in need thereof.

In another aspect, the present invention relies on methods for treating patients in need thereof, said method comprising at least one of the following steps:
(a) Determining specific antigen markers present at the surface of patients tumors biopsies;
(b) providing a population of engineered primary immune cells engineered by one of the methods of the present invention previously described expressing a CAR directed against said specific antigen markers;
(c) Administrating said engineered population of engineered primary immune cells to said patient,

Generally, said populations of cells mainly comprises CD4 and CD8 positive immune cells, such as T-cells, which can undergo robust in vivo T cell expansion and can persist for an extended amount of time in-vitro and in-vivo.

The treatments involving the engineered primary immune cells according to the present invention can be ameliorating, curative or prophylactic. It may be either part of an autologous immunotherapy or part of an allogenic immunotherapy treatment. By autologous, it is meant that cells, cell line or population of cells used for treating patients are originating from said patient or from a Human Leucocyte Antigen (HLA) compatible donor. By allogeneic is meant that the cells or population of cells used for treating patients are not originating from said patient but from a donor.

In another embodiment, said isolated cell according to the invention or cell line derived from said isolated cell can be used for the treatment of liquid tumors, and preferably of T-cell acute lymphoblastic leukemia.

Adult tumors/cancers and pediatric tumors/cancers are also included.

The treatment with the engineered immune cells according to the invention may be in combination with one or more therapies against cancer selected from the group of antibodies therapy, chemotherapy, cytokines therapy, dendritic cell therapy, gene therapy, hormone therapy, laser light therapy and radiation therapy.

According to a preferred embodiment of the invention, said treatment can be administrated into patients undergoing an immunosuppressive treatment. Indeed, the present invention preferably relies on cells or population of cells, which have been made resistant to at least one immunosuppressive agent due to the inactivation of a gene encoding a receptor for such immunosuppressive agent. In this aspect, the immunosuppressive treatment should help the selection and expansion of the T-cells according to the invention within the patient.

The administration of the cells or population of cells according to the present invention may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous or intralymphatic injection, or intraperitoneally. In one embodiment, the cell compositions of the present invention are preferably administered by intravenous injection.

The administration of the cells or population of cells can consist of the administration of 10⁴-10⁹ cells per kg body weight, preferably 10⁵ to 10⁶ cells/kg body weight including all integer values of cell numbers within those ranges. The present invention thus can provide more than 10, generally more than 50, more generally more than 100 and usually more than 1000 doses comprising between 10⁶ to 10⁸ gene edited cells originating from a single donor's or patient's sampling.

The cells or population of cells can be administrated in one or more doses. In another embodiment, said effective amount of cells are administrated as a single dose. In another embodiment, said effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions within the skill of the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, said effective amount of cells or composition comprising those cells are administrated parenterally. Said administration can be an intravenous administration. Said administration can be directly done by injection within a tumor.

In certain embodiments of the present invention, cells are administered to a patient in conjunction with (e.g., before, simultaneously or following) any number of relevant treatment modalities, including but not limited to treatment with agents such as antiviral therapy, cidofovir and interleukin-2, Cytarabine (also known as ARA-C) or nataliziimab treatment for MS patients or efaliztimab treatment for psoriasis patients or other treatments for PML patients. In further embodiments, the T cells of the invention may be used in combination with chemotherapy, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludaribine, cyclosporin, FK506, rapamycin, mycoplienolic acid, steroids, FR901228, cytokines, and irradiation. These drugs inhibit either the calcium dependent phosphatase calcineurin (cyclosporine and FK506) or inhibit the p70S6 kinase that is important for growth factor induced signaling (rapamycin) (Henderson, Naya et al. 1991; Liu, Albers et al. 1992; Bierer, Hollander et al. 1993). In a further embodiment, the cell compositions of the present invention are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH, In another embodiment, the cell compositions of the present invention are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

When CARs are expressed in the immune cells or populations of immune cells according to the present invention, the preferred CARs are those targeting at least one antigen selected from CD22, CD38, CD123, CS1, HSP70, ROR1, GD3, and CLL1.

The engineered immune cells according to the present invention endowed with a CAR or a modified TCR targeting CD22 are preferably used for treating leukemia, such as acute lymphoblastic leukemia (ALL), those with a CAR or a modified TCR targeting CD38 are preferably used for treating leukemia such as T-cell acute lymphoblastic leukemia (T-ALL) or multiple myeloma (MM), those with a CAR or a modified TCR targeting CD123 are preferably used for treating leukemia, such as acute myeloid leukemia (AML), and blastic plasmacytoid dendritic cells neoplasm (BPDCN), those with a CAR or a modified TCR targeting CS1 are preferably used for treating multiple myeloma (MM).

The invention is also suited for allogenic immunotherapy, insofar as it is compatible with any known methods in the art intended to reduce TCR expression in immune cells, such as T-cells, typically obtained from donors, such as gene inactivation by using a rare-cutting endonuclease. Such methods enables the production of immune cells with reduced alloreactivity. The resultant modified immune cells may be pooled and administrated to one or several patients, being made available as an “off the shelf” therapeutic product as described by Poirot et al. [Poirot, L. et al. (2015) Multiplex Genome-Edited T-cell Manufacturing Platform for “Off-the-Shelf” Adoptive T-cell Immunotherapies. Cancer Res. 75(18)]. Gene targeting insertion at the TCR locus of a chimeric polynucleotide according to the present invention can also lead to TCR gene inactivation and provide with engineered allogeneic (primary) immune cells which are less alloreactive.

According to certain embodiments, the immune cell(s) or composition is for use in the treatment of a cancer, and more particularly for use in the treatment of a solid or liquid tumor. According to particular embodiments, the immune cell(s) or composition is for use in the treatment of a solid tumor. According to other particular embodiments, the immune cell(s) or composition is for use in the treatment of a liquid tumor.

According to particular embodiments, the immune cell(s) or composition is for use in the treatment of a cancer selected from the group consisting of lung cancer, small lung cancer, breast cancer, uterine cancer, prostate cancer, kidney cancer, colon cancer, liver cancer, pancreatic cancer, and skin cancer. According to more particular embodiments, the immune cell(s) or composition is for use in the treatment of lung cancer. According to other more particular embodiments, the immune cell(s) or composition is for use in the treatment of small lung cancer. According to other more particular embodiments, the immune cell(s) or composition is for use in the treatment of breast cancer. According to other more particular embodiments, the immune cell(s) or composition is for use in the treatment of uterine cancer. According to other more particular embodiments, the immune cell(s) or composition is for use in the treatment of prostate cancer. According to other more particular embodiments, the immune cell(s) or composition is for use in the treatment of kidney cancer. According to other more particular embodiments, the immune cell(s) or composition is for use in the treatment of colon cancer. According to other more particular embodiments, the immune cell(s) or composition is for use in the treatment of liver cancer. According to other more particular embodiments, the immune cell(s) or composition is for use in the treatment of pancreatic cancer. According to other more particular embodiments, the immune cell(s) or composition is for use in the treatment of skin cancer.

According to other particular embodiments, the immune cell(s) or composition is for use in the treatment of a sarcoma.

According to other particular embodiments, the immune cell(s) or composition is for use in the treatment of a carcinoma. According to more particular embodiments, the immune cell or composition is for use in the treatment of renal, lung or colon carcinoma.

According to other particular embodiments, the immune cell(s) or composition is for use in the treatment of leukemia, such as acute lymphoblastic leukemia (ALL), acute myeloid leukemia (AML), chronic lymphocytic leukemia (CLL), chronic myelogenous leukemia (CML), and chronic myelomonocystic leukemia (CMML). According to more particular embodiments, the immune cell(s) or composition is for use in the treatment of acute lymphoblastic leukemia (ALL). According to other more particular embodiments, the immune cell(s) or composition is for use in the treatment of acute myeloid leukemia (AML). According to other more particular embodiments, the immune cell(s) or composition is for use in the treatment of chronic lymphocytic leukemia (CLL). According to other more particular embodiments, the immune cell(s) or composition is for use in the treatment of chronic myelogenous leukemia (CML). According to other more particular embodiments, the immune cell(s) or composition is for use in the treatment of chronic myelomonocystic leukemia (CMML).

According to other particular embodiments, the immune cell(s) or composition is for use in the treatment of lymphoma, such as B-cell lymphoma. According to more particular embodiments, the immune cell(s) or composition is for use in the treatment of primary CNS lymphoma. According to other more particular embodiments, the immune cell(s) or composition is for use in the treatment of Hodgkin's lymphoma. According to other more particular embodiments, the immune cell(s) or composition is for use in the treatment of Non-Hodgkin's lymphoma. According to more particular embodiments, the immune cell(s) or composition is for use in the treatment of diffuse large B cell lymphoma (DLBCL). According to other more particular embodiments, the immune cell(s) or composition is for use in the treatment of Follicular lymphoma. According to other more particular embodiments, the immune cell(s) or composition is for use in the treatment of marginal zone lymphoma (MZL). According to other more particular embodiments, the immune cell(s) or composition is for use in the treatment of Mucosa-Associated Lymphatic Tissue lymphoma (MALT). According to other more particular embodiments, the immune cell(s) or composition is for use in the treatment of small cell lymphocytic lymphoma. According to other more particular embodiments, the immune cell(s) or composition is for use in the treatment of mantle cell lymphoma (MCL). According to other more particular embodiments, the immune cell(s) or composition is for use in the treatment of Burkitt lymphoma. According to other more particular embodiments, the immune cell(s) or composition is for use in the treatment of primary mediastinal (thymic) large B-cell lymphoma. According to other more particular embodiments, the immune cell(s) or composition is for use in the treatment of Waldenstrom macroglobulinemia. According to other more particular embodiments, the immune cell(s) or composition is for use in the treatment of nodal marginal zone B cell lymphoma (NMZL). According to other more particular embodiments, the immune cell(s) or composition is for use in the treatment of splenic marginal zone lymphoma (SMZL). According to other more particular embodiments, the immune cell(s) or composition is for use in the treatment of intravascular large B-cell lymphoma. According to other more particular embodiments, the immune cell(s) or composition is for use in the treatment of Primary effusion lymphoma. According to other more particular embodiments, the immune cell(s) or composition is for use in the treatment of lymphomatoid granulomatosis. According to other more particular embodiments, the immune cell(s) or composition is for use in the treatment of T cell/histiocyte-rich large B-cell lymphoma. According to other more particular embodiments, the immune cell(s) or composition is for use in the treatment of primary diffuse large B-cell lymphoma of the CNS (Central Nervous System). According to other more particular embodiments, the immune cell(s) or composition is for use in the treatment of primary cutaneous diffuse large B-cell lymphoma. According to other more particular embodiments, the immune cell(s) or composition is for use in the treatment of EBV positive diffuse large B-cell lymphoma of the elderly. According to other more particular embodiments, the immune cell(s) or composition is for use in the treatment of diffuse large B-cell lymphoma associated with inflammation. According to other more particular embodiments, the immune cell(s) or composition is for use in the treatment of ALK-positive large B-cell lymphoma. According to other more particular embodiments, the immune cell(s) or composition is for use in the treatment of plasmablastic lymphoma. According to other more particular embodiments, the immune cell(s) or composition is for use in the treatment of Large B-cell lymphoma arising in HHV8-associated multicentric Castleman disease.

According to certain embodiments, the immune cell(s) or composition is for use in the treatment of a viral infection, such as an HIV infection or HBV infection.

According to certain embodiment, the immune cell of originates from a patient, e.g. a human patient, to be treated. According to certain other embodiment, the immune cell originates from at least one donor.

The treatment can take place in combination with one or more therapies selected from the group of antibodies therapy, chemotherapy, cytokines therapy, dendritic cell therapy, gene therapy, hormone therapy, laser light therapy and radiation therapy.

According to certain embodiments, immune cells of the invention can undergo robust in vivo immune cell expansion upon administration to a patient, and can persist in the body fluids for an extended amount of time, preferably for a week, more preferably for 2 weeks, even more preferably for at least one month. Although the immune cells according to the invention are expected to persist during these periods, their life span into the patient's body are intended not to exceed a year, preferably 6 months, more preferably 2 months, and even more preferably one month.

The administration of the immune cells or composition according to the present invention may be carried out in any convenient manner, including by aerosol inhalation, injection, ingestion, transfusion, implantation or transplantation. The immune cells or composition described herein may be administered to a patient subcutaneously, intradermally, intratumorally, intranodally, intramedullary, intramuscularly, by intravenous or intralymphatic injection, or intraperitoneally.

According to certain embodiments, the immune cells or composition are/is administered by intravenous injection.

According to other certain embodiments, the immune cell(s) or composition is administrated parenterally.

According to certain other embodiments, the immune cell(s) or composition is administered intratumorally. Said administration can be done by injection directly into a tumor or adjacent thereto.

The administration of the cells or population of cells can consist of the administration of 104-109 cells per kg body weight, preferably 10⁵ to 10⁶ cells/kg body weight including all integer values of cell numbers within those ranges. The cells or population of cells can be administrated in one or more doses. In another embodiment, said effective amount of cells are administrated as a single dose. In another embodiment, said effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions within the skill of the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

According to certain embodiments, immune cells are administered to a patient in conjunction with (e.g., before, simultaneously or following) any number of relevant treatment modalities, including but not limited to treatment with agents such as antiviral therapy, cidofovir and interleukin-2, Cytarabine (also known as ARA-C) or nataliziimab treatment for MS patients or efaliztimab treatment for psoriasis patients or other treatments for PML patients. In further embodiments, the T cells of the invention may be used in combination with chemotherapy, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludaribine, cyclosporin, FK506, rapamycin, mycoplienolic acid, steroids, FR901228, cytokines, and irradiation. These drugs inhibit either the calcium dependent phosphatase calcineurin (cyclosporine and FK506) or inhibit the p70S6 kinase that is important for growth factor induced signaling (rapamycin). In a further embodiment, the cell compositions of the present invention are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH, In another embodiment, the cell compositions of the present invention are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of the expanded genetically engineered immune cells of the present invention. In an additional embodiment, expanded cells are administered before or following surgery.

Also encompassed within this aspect of the invention are methods for treating a patient in need thereof, comprising a) providing at least one immune cell of the present invention, preferably a population of said immune cell; and b) administering said immune cell or population to said patient.

Also encompassed are method of treatments comprising the co-administration of engineered immune cells endowed with a chimeric polypeptide as per the present invention with a dose of a protease inhibitor, especially Asunaprevir at a dose ranging from 10 to 600 mg a day by oral administration, preferably 40 to 400, more preferably 50 to 200 mg/day for an adult patient.

Also encompassed within this aspect of the invention are methods for preparing a medicament using at least one immune cell of the present invention, and preferably a population of said immune cell. Accordingly, the present invention provides the use of at least one immune cell of the present invention, and preferably a population of said immune cell, in the manufacture of a medicament. Preferably, such medicament is for use in the treatment of a disease as specified above.

### Other Definitions

Amino acid residues in a polypeptide sequence are designated herein according to the one-letter code, in which, for example, Q means Gin or Glutamine residue, R means Arg or Arginine residue and D means Asp or Aspartic acid residue.
Amino acid substitution means the replacement of one amino acid residue with another, for instance the replacement of an Arginine residue with a Glutamine residue in a peptide sequence is an amino acid substitution.
Nucleotides are designated as follows: one-letter code is used for designating the base of a nucleoside: a is adenine, t is thymine, c is cytosine, and g is guanine. For the degenerated nucleotides, r represents g or a (purine nucleotides), k represents g or t, s represents g or c, w represents a or t, m represents a or c, y represents t or c (pyrimidine nucleotides), d represents g, a or t, v represents g, a or c, b represents g, t or c, h represents a, t or c, and n represents g, a, t or c.
“As used herein, “nucleic acid” or “polynucleotides” refers to nucleotides and/or polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Nucleic acids can be either single stranded or double stranded.
The term “endonuclease” refers to any wild-type or variant enzyme capable of catalyzing the hydrolysis (cleavage) of bonds between nucleic acids within a DNA or RNA molecule, preferably a DNA molecule. Endonucleases do not cleave the DNA or RNA molecule irrespective of its sequence, but recognize and cleave the DNA or RNA molecule at specific polynucleotide sequences. Endonucleases can be classified as rare-cutting endonucleases when having typically a polynucleotide recognition site greater than 10 base pairs (bp) in length, more preferably of 14-55 bp. Rare-cutting endonucleases significantly increase homologous recombination by inducing DNA double-strand breaks (DSBs) at a defined locus thereby allowing gene repair or gene insertion therapies (Pingoud, A. and G. H. Silva (2007). Precision genome surgery. Nat. Biotechnol. 25(7): 743-4.). Examples of rare-cutting endonucleases are homing endonuclease as described for instance by Arnould S., et al. (WO2004067736), zing finger nucleases (ZFN) as described, for instance, by Urnov F., et al. [Highly efficient endogenous human gene correction using designed zinc-finger nucleases (2005) Nature 435:646-651], a TALE-Nuclease as described, for instance, by Mussolino et al. [A novel TALE nuclease scaffold enables high genome editing activity in combination with low toxicity (2011) Nucl. Acids Res. 39(21):9283-9293], MegaTAL nucleases as described, for instance by Boissel et al. [MegaTALs: a rare-cleaving nuclease architecture for therapeutic genome engineering (2013) Nucleic Acids Research 42 (4):2591-2601] or RNA-guided endonuclease, such as Cas9 or Cpf1, as per, inter alia, the teaching by Doudna, J. et al., [The new frontier of genome engineering with CRISPR-Cas9 (2014) Science 346 (6213):1077)] and Zetsche, B. et al. [Cpf1 Is a Single RNA-Guided Endonuclease of a Class 2 CRISPR-Cas System (2015) Cell 163(3): 759-771] the teaching of which is incorporated herein by reference.
The term “cleavage” refers to the breakage of the covalent backbone of a polynucleotide. Cleavage can be initiated by a variety of methods including, but not limited to, enzymatic or chemical hydrolysis of a phosphodiester bond, typically using an endonuclease. Both single-stranded cleavage and double-stranded cleavage are possible, and double-stranded cleavage can occur as a result of two distinct single-stranded cleavage events. Double stranded DNA, RNA, or DNA/RNA hybrid cleavage can result in the production of either blunt ends or staggered ends.
By “DNA target”, “DNA target sequence”, “target DNA sequence”, “nucleic acid target sequence”, “target sequence”, or “processing site” is intended a polynucleotide sequence that can be targeted and processed by a rare-cutting endonuclease according to the present invention. These terms refer to a specific DNA location, preferably a genomic location in a cell, but also a portion of genetic material that can exist independently to the main body of genetic material such as plasmids, episomes, virus, transposons or in organelles such as mitochondria as non-limiting example. As non-limiting examples of RNA guided target sequences, are those genome sequences that can hybridize the guide RNA which directs the RNA guided endonuclease to a desired locus.
By “mutation” is intended the substitution, deletion, insertion of up to one, two, three, four, five, six, seven, eight, nine, ten, eleven, twelve, thirteen, fourteen, fifteen, twenty, twenty five, thirty, fourty, fifty, or more nucleotides/amino acids in a polynucleotide (cDNA, gene) or a polypeptide sequence. The mutation can affect the coding sequence of a gene or its regulatory sequence. It may also affect the structure of the genomic sequence or the structure/stability of the encoded mRNA.
By “vector” is meant a nucleic acid molecule capable of transporting another nucleic acid to which it has been linked. A “vector” in the present invention includes, but is not limited to, a viral vector, a plasmid, a RNA vector or a linear or circular DNA or RNA molecule which may consists of a chromosomal, non chromosomal, semi-synthetic or synthetic nucleic acids. Preferred vectors are those capable of autonomous replication (episomal vector) and/or expression of nucleic acids to which they are linked (expression vectors). Large numbers of suitable vectors are known to those of skill in the art and commercially available. Viral vectors include retrovirus, adenovirus, parvovirus (e. g. adenoassociated viruses (AAV), coronavirus, negative strand RNA viruses such as orthomyxovirus (e. g., influenza virus), rhabdovirus (e. g., rabies and vesicular stomatitis virus), paramyxovirus (e. g. measles and Sendai), positive strand RNA viruses such as picornavirus and alphavirus, and double-stranded DNA viruses including adenovirus, herpesvirus (e. g., Herpes Simplex virus types 1 and 2, Epstein-Barr virus, cytomegalovirus), and poxvirus (e. g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996).
As used herein, the term “locus” is the specific physical location of a DNA sequence (e.g. of a gene) into a genome. The term “locus” can refer to the specific physical location of a rare-cutting endonuclease target sequence on a chromosome or on an infection agent's genome sequence. Such a locus can comprise a target sequence that is recognized and/or cleaved by a sequence-specific endonuclease according to the invention. It is understood that the locus of interest of the present invention can not only qualify a nucleic acid sequence that exists in the main body of genetic material (i.e. in a chromosome) of a cell but also a portion of genetic material that can exist independently to said main body of genetic material such as plasmids, episomes, virus, transposons or in organelles such as mitochondria as non-limiting examples.

With “cytolytic activity” it is meant the percentage of cell lysis of target cells conferred by an immune cell expressing said CAR.

A method for determining the cytotoxicity is described below:

With Adherent Target Cells:

2×10⁴ specific target antigen (STA)-positive or STA-negative cells are seeded in 0.1 ml per well in a 96 well plate. The day after the plating, the STA-positive and the STA-negative cells are labeled with CellTrace CFSE and co-cultured with 4×10⁵ T cells for 4 hours. The cells are then harvested, stained with a fixable viability dye (eBioscience) and analyzed using the MACSQuant flow cytometer (Miltenyi).

The percentage of specific lysis is calculated using the following formula:

$$\% cell lysis = 100 \% - \frac{\frac{\begin{matrix}\% viable target cells upon \\ coculture with CAR modified T cells\end{matrix}}{\begin{matrix}\% viable control cells upon \\ coculture with CAR modified T cells\end{matrix}}}{\frac{\begin{matrix}\% viable target cells upon \\ coculture with non modified T cells\end{matrix}}{\begin{matrix}\% viable control cells \\ coculture with non modified T cells\end{matrix}}}$$

With Suspension Target Cells:

STA-positive and STA-negative cells are respectively labeled with CellTrace CFSE and CellTrace Violet. About 2×10⁴ ROR1-positive cells are co-cultured with 2×10⁴ STA-negative cells with 4×10⁵ T cells in 0.1 ml per well in a 96-well plate. After a 4 hour incubation, the cells are harvested and stained with a fixable viability dye (eBioscience) and analyzed using the MACSQuant flow cytometer (Miltenyi).

The percentage of specific lysis is calculated using the previous formula.

“Specific target antigen (STA)-positive cells” means cells which express the target antigen for which the chimeric antigen receptor shows specificity, whereas “STA-negative cells” means cells which do not express the specific target antigen. By way of a non-limiting example, if the CAR is directed against CD19, the specific target antigen is thus CD19. Accordingly, CD19-positive and CD19-negative cells are to be used to determine the cytolytic activity.

Hence, the above-described cytotoxicity assay will have to be adapted to the respective target cells depending on the antigen-specificity of the chimeric antigen receptor expressed by the immune cell.

Similar methods for assaying the cytolytic activity are also described in, e.g., Valton et al. (2015) or Poirot et al. (2015).

According to certain embodiments, a chimeric antigen receptor according to the present invention confers a modulated cytolytic activity to an immune cell expressing same in the presence of a corresponding multimerizing ligand compared to the cytolytic activity of said immune cell in the absence of the multimerizing ligand.

By “increased cytolytic activity” it is meant that the % cell lysis of target cells conferred by the immune cell expressing said CAR increases by at least 10%, such as at least 20%, at least 30%, at least 40%, at least 50%, at least 60%, at least 70%, at least 80%, at least 90% or at least 100%, in the presence of the multimerizing ligand compared to the % cell lysis of target cells conferred by the immune cell in the absence of the multimerizing ligand.
“identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting. For example, polypeptides having at least 70%, 85%, 90%, 95%, 98% or 99% identity to specific polypeptides described herein and preferably exhibiting substantially the same functions, as well as polynucleotide encoding such polypeptides, are contemplated.
The term “subject” or “patient” as used herein includes all members of the animal kingdom including non-human primates and humans.
The above written description of the invention provides a manner and process of making and using it such that any person skilled in this art is enabled to make and use the same, this enablement being provided in particular for the subject matter of the appended claims, which make up a part of the original description.

Where a numerical limit or range is stated herein, the endpoints are included. Also, all values and subranges within a numerical limit or range are specifically included as if explicitly written out.

Having generally described this invention, a further understanding can be obtained by reference to certain specific examples, which are provided herein for purposes of illustration only, and are not intended to limit the scope of the claimed invention.

### EXAMPLES

### Example 1

Polynucleotide sequences have been assembled into lentiviral vectors in view of transducing primary T-cells expressing CARs with small molecule based degradation properties.

Lentiviral Vectors Encoding CARs with C-Terminal Small Molecule Based Controlled Degradation Moiety

CARs have been designed comprising a self-excising degron as per the following structure (from N to C-terminus):

(1) a signal peptide for targeting to the cell surface derived from the T-cell surface glycoprotein CD8 alpha chain (SEQ ID NO: 21),

(2) an antigen binding domain (ScFv) respectively derived from anti-CD123 and anti-CD22 antibodies (SEQ ID NO: 22 and SEQ ID NO: 23),

(3) a stalk (or hinge) domain derived from the T-cell surface glycoprotein CD8 alpha chain (SEQ ID NO: 24),

(4) a transmembrane domain derived from the T-cell surface glycoprotein CD8 alpha chain (SEQ ID NO: 25) and

(5) an intracellular domain (SEQ ID NO: 26) comprising itself a co-stimulation moiety derived from the Tumor necrosis factor receptor superfamily member 9 (SEQ ID NO: 27) and an ITAM based activation moiety derived from T-cell surface glycoprotein CD3 zeta chain (SEQ ID NO: 28).

The above (1) to (6) sequences form the active CARs to be expressed at the surface of the immune cells, which are fused at their 3′ end (C-terminal end) to the following polynucleotides sequences forming the self-excising degron:

(6) a protease target site (SEQ ID NO: 29),

(7) a linker/tag (SEQ ID NO: 30),

(8) a protease derived from the NS3 protease domain (SEQ ID NO: 31),

(9) a degron derived from the NS3 protease domain or from the NS4A protein (SEQ ID: 32) respectively leading to pCLS29306 (C-ter degronCAR anti-CD123—SEQ ID NO: 33) and pCLS30066 (C-ter degronCAR anti-CD22—SEQ ID NO: 34).

The resulting polynucleotide sequences (shown in FIG. 4A) are cloned into lentiviral production plasmids (genome plasmid) under the control of a SFFV promoters (SEQ ID NO: 15) by using standard molecular biology techniques such as PCR (Agilent Herculase II fusion Enzyme cat #600677), enzymatic restriction digestions (New England Biolabs or ThermoFisher), ligations (T4 DNA ligase cat # EL0011) and bacterial transformations (XL1b, Agilent cat #200236 or One shot Stbl3, ThermoFisher cat # C7373-03) according to the manufacturer instructions.

The integrity of the CAR fusion sequences were verified by Sanger DNA sequencing (GenScript). Plasmids used for lentiviral particules preparation were obtained from One shot Stbl3 transformation and purified using Nucleobond Maxi Xtra EF kits (Macherey-Nagel cat #740424.50). Lentiviral particles are generated in 293FT cells (ThermoFisher) cultured in RPMI 1640 Medium (ThermoFisher cat # SH30027FS) supplemented with 10% FBS (Gibco cat #10091-148), 1% HEPES (Gibco cat #15630-80), 1% L-Glutamine (Gibco cat #35050-61) and 1% Penicilin/Streptomycin (Gibco cat #15070-063) using Opti-MEM medium (Gibco cat #31985-062) and Lipofectamine 2000 (Thermo Fisher cat #11668-019) according to standard transfection procedures. Supernatants containing the viral particles are recovered and concentrated by ultracentrifugation 48 and/or 72 hours post transfection.

Lentiviral Vectors Encoding CARs with N-Terminal Small Molecule Based Controlled Degradation Moiety

Further CARs have been constructed comprising a CAR region and a self-excising degron at their N-terminus having the following structure:

(1) a signal peptide for targeting to the cell surface derived from the T-cell surface glycoprotein CD8 alpha chain (SEQ ID NO: 21),

(2) an antigen binding domain (ScFv) respectively derived from anti-CD22 antibodies (SEQ ID NO: 23),

(3) a stalk (or hinge) domain derived from the T-cell surface glycoprotein CD8 alpha chain (SEQ ID NO: 24),

(4) a transmembrane domain derived from the T-cell surface glycoprotein CD8 alpha chain (SEQ ID NO: 25) and

(5) an intracellular domain (SEQ ID NO: 26) comprising itself a co-stimulation moiety derived from the Tumor necrosis factor receptor superfamily member 9 (SEQ ID NO: 27) and an ITAM based activation moiety derived from T-cell surface glycoprotein CD3 zeta chain (SEQ ID NO: 28).

The above (1) to (6) polynucleotide sequences being fused at their 5′ end (N-terminal end) to the following polynucleotides sequences forming the self-excising degron:
a protease target site (SEQ ID NO: 29),
a linker/tag (SEQ ID NO: 30),
a protease derived from the NS3 protease domain (SEQ ID NO: 31),
a degron derived from the NS3 protease domain or from the NS4A protein (SEQ ID: 27) leading to pCLS30018 (N-ter degron-CAR anti-CD22 SEQ ID NO: 28).

The resulting polynucleotide sequences (shown in FIG. 4B) are cloned into lentiviral production plasmids (genome plasmid) under the control of a SFFV promoters (SEQ ID NO: 35) by using standard molecular biology techniques such as PCR (Agilent Herculase II fusion Enzyme cat #600677), enzymatic restriction digestions (New England Biolabs or ThermoFisher), ligations (T4 DNA ligase cat # EL0011) and bacterial transformations (XL1b, Agilent cat #200236 or One shot Stbl3, ThermoFisher cat # C7373-03) according to the manufacturer instructions.

The integrity of the CAR fusion sequences were verified by Sanger DNA sequencing (GenScript). Plasmids used for lentiviral particules preparation were obtained from One shot Stbl3 transformation and purified using Nucleobond Maxi Xtra EF kits (Macherey-Nagel cat #740424.50). Lentiviral particles are generated in 293FT cells (ThermoFisher) cultured in RPMI 1640 Medium (ThermoFisher cat # SH30027FS) supplemented with 10% FBS (Gibco cat #10091-148), 1% HEPES (Gibco cat #15630-80), 1% L-Glutamine (Gibco cat #35050-61) and 1% Penicilin/Streptomycin (Gibco cat #15070-063) using Opti-MEM medium (Gibco cat #31985-062) and Lipofectamine 2000 (Thermo Fisher cat #11668-019) according to standard transfection procedures. Supernatants containing the viral particles are recovered and concentrated by ultracentrifugation 48 and/or 72 hours post transfection.

The polynucleotides and corresponding polypeptide sequences used in the Examples are detailed in Table 10 below.

TABLE 10
polynucleotide and polypeptide sequences used in the examples 1 to 4

| Designation | SEQ ID # | Polynucleotide/polypeptide sequences |
|---|---|---|
| CD8 signal | 21 | MALPVTALLLPLALLLHAARP |
| sequence | | |
| CD123 targeting | 22 | QIQLVQSGPELKKPGETVKISCKASGYIFTNYGMNWVKQAPGKSFK |
| scFv | | WMGWINTYTGESTYSADFKGRFAFSLETSASTAYLHINDLKNEDTA |
| | | TYFCARSGGYDPMDYWGQGTSVTVSSGGGGSGGGGSGGGGSDIVLT |
| | | QSPASLAVSLGQRATISCRASESVDNYGNTFMHWYQQKPGQPPKLL |
| | | IYRASNLESGIPARFSGSGSRTDFTLTINPVEADDVATYYCQQSNE |
| | | DPPTFGAGTKLELKRSDPGSGGGGSCPYSNPSLCSGGGGSCPYSNP |
| | | SLCAP |
| CD22 targeting | 23 | QVQLQQSGPGLVKPSQTLSLTCAISGDSVSSNSAAWNWIRQSPSRG |
| scFv | | LEWLGRTYYRSKWYNDYAVSVKSRITINPDTSKNQFSLQLNSVTPE |
| | | DTAVYYCAREVTGDLEDAFDIWGQGTMVTVSSGGGGSGGGGSGGGG |
| | | SDIQMTQSPSSLSASVGDRVTITCRASQTIWSYLNWYQQRPGKAPN |
| | | LLIYAASSLQSGVPSRFSGRGSGTDFTLTISSLQAEDFATYYCQQS |
| | | YSIPQTFGQGTKLEIKAP |
| cd8 hinge | 24 | TTTPAPRPPTPAPTIASQPLSLRPEACRPAAGGAVHTRGLDFACD |
| cd8 | 25 | IYIWAPLAGTCGVLLLSLVITLYC |
| transmembrane | | |
| Intracellular | 26 | RRGRKKLLYIFKQPFMRPVQTTQEEDGCSCRFPEEEEGGCELRVKF |
| domain | | SRSADAPAYQQGQNQLYNELNLGRREEYDVLDKRRGRDPEMGGKPR |
| | | RKNPQEGLYNELQKDKMAEAYSEIGMKGERRRGKGHDGLYQGLSTA |
| | | TKDTYDALHMQALPPR |
| 4-1BB costim | 27 | RGRKKLLYIFKQPFMRPVQTTQEEDGCSCRFPEEEEGGCEL |
| CD3 activation | 28 | RVKFSRSADAPAYQQGQNQLYNELNLGRREEYDVLDKRRGRDPEMG |
| | | GKPRRKNPQEGLYNELQKDKMAEAYSEIGMKGERRRGKGHDGLYQG |
| | | LSTATKDTYDALHMQALPPR |
| NS3 protease | 29 | DEMEECSQHL |
| target site | | |
| linker/tag | 30 | PGAGSSGDIMDYKDDDDKGSSGTGSGSGTS |
| NS3 Protease | 31 | APITAYAQQTRGLLGCIITSLTGRDKNQVEGEVQIVSTATQTFLAT |
| domain | | CINGVCWAVYHGAGTRTIASPKGPVIQMYTNVDQDLVGWPAPQGSR |
| | | SLTPCTCGSSDLYLVTRHADVIPVRRRGDSRGSLLSPRPISYLKGS |
| | | SGGPLLCPAGHAVGLFRAAVCTRGVAKAVDFIPVENLETTMRSPVF |
| | | TD |
| NS3/NS4 degron | 32 | NSSPPAVTLTHPITKIDTKYIMTCMSADLEVVTSTWVLVGGVLAAL |
| | | AAYCLSTGCVVIVGRIVLSGKPAIIPDREVLY |
| pCLS29306 | 33 | MALPVTALLLPLALLLHAARPQIQLVQSGPELKKPGETVKISCKAS |
| (targeting CD123) | | GYIFTNYGMNWVKQAPGKSFKWMGWINTYTGESTYSADFKGRFAFS |
| | | LETSASTAYLHINDLKNEDTATYFCARSGGYDPMDYWGQGTSVTVS |
| | | SGGGGSGGGGSGGGGSDIVLTQSPASLAVSLGQRATISCRASESVD |
| | | NYGNTFMHWYQQKPGQPPKLLIYRASNLESGIPARFSGSGSRTDFT |
| | | LTINPVEADDVATYYCQQSNEDPPTFGAGTKLELKRSDPGSGGGGS |
| | | CPYSNPSLCSGGGGSCPYSNPSLCAPTTTPAPRPPTPAPTIASQPL |
| | | SLRPEACRPAAGGAVHTRGLDFACDIYIWAPLAGTCGVLLLSLVIT |
| | | LYCRRGRKKLLYIFKQPFMRPVQTTQEEDGCSCRFPEEEEGGCELR |
| | | VKFSRSADAPAYQQGQNQLYNELNLGRREEYDVLDKRRGRDPEMGG |
| | | KPRRKNPQEGLYNELQKDKMAEAYSEIGMKGERRRGKGHDGLYQGL |
| | | STATKDTYDALHMQALPPRSGDEMEECSQHLPGAGSSGDIMDYKDD |
| | | DDKGSSGTGSGSGTSAPITAYAQQTRGLLGCIITSLTGRDKNQVEG |
| | | EVQIVSTATQTFLATCINGVCWAVYHGAGTRTIASPKGPVIQMYTN |
| | | VDQDLVGWPAPQGSRSLTPCTCGSSDLYLVTRHADVIPVRRRGDSR |
| | | GSLLSPRPISYLKGSSGGPLLCPAGHAVGLFRAAVCTRGVAKAVDF |
| | | IPVENLETTMRSPVFTDNSSPPAVTLTHPITKIDTKYIMTCMSADL |
| | | EVVTSTWVLVGGVLAALAAYCLSTGCVVIVGRIVLSGKPAIIPDRE |
| | | VLYE |
| pCLS30066 | 34 | MALPVTALLLPLALLLHAARPQVQLQQSGPGLVKPSQTLSLTCAIS |
| (targeting CD22) | | GDSVSSNSAAWNWIRQSPSRGLEWLGRTYYRSKWYNDYAVSVKSRI |
| | | TINPDTSKNQFSLQLNSVTPEDTAVYYCAREVTGDLEDAFDIWGQG |
| | | TMVTVSSGGGGSGGGGSGGGGSDIQMTQSPSSLSASVGDRVTITCR |
| | | ASQTIWSYLNWYQQRPGKAPNLLIYAASSLQSGVPSRFSGRGSGTD |
| | | FTLTISSLQAEDFATYYCQQSYSIPQTFGQGTKLEIKAPTTTPAPR |
| | | PPTPAPTIASQPLSLRPEACRPAAGGAVHTRGLDFACDIYIWAPLA |
| | | GTCGVLLLSLVITLYCRRGRKKLLYIFKQPFMRPVQTTQEEDGCSC |
| | | RFPEEEEGGCELRVKFSRSADAPAYQQGQNQLYNELNLGRREEYDV |
| | | LDKRRGRDPEMGGKPRRKNPQEGLYNELQKDKMAEAYSEIGMKGER |
| | | RRGKGHDGLYQGLSTATKDTYDALHMQALPPRSGDEMEECSQHLPG |
| | | AGSSGDIMDYKDDDDKGSSGTGSGSGTSAPITAYAQQTRGLLGCII |
| | | TSLTGRDKNQVEGEVQIVSTATQTFLATCINGVCWAVYHGAGTRTI |
| | | ASPKGPVIQMYTNVDQDLVGWPAPQGSRSLTPCTCGSSDLYLVTRH |
| | | ADVIPVRRRGDSRGSLLSPRPISYLKGSSGGPLLCPAGHAVGLFRA |
| | | AVCTRGVAKAVDFIPVENLETTMRSPVFTDNSSPPAVTLTHPITKI |
| | | DTKYIMTCMSADLEVVTSTWVLVGGVLAALAAYCLSTGCVVIVGRI |
| | | VLSGKPAIIPDREVLY |
| SFFV promoter | 35 | GATAAAATAAAAGATTTTATTTAGTCTCCAGAAAAAGGGGGGAATG |
| | | AAAGACCCCACCTGTAGGTTTGGCAAGCTAGCTGCAGTAACGCCAT |
| | | TTTGCAAGGCATGGAAAAATACCAAACCAAGAATAGAGAAGTTCAG |
| | | ATCAAGGGCGGGTACATGAAAATAGCTAACGTTGGGCCAAACAGGA |
| | | TATCTGCGGTGAGCAGTTTCGGCCCCGGCCCGGGGCCAAGAACAGA |
| | | TGGTCACCGCAGTTTCGGCCCCGGCCCGAGGCCAAGAACAGATGGT |
| | | CCCCAGATATGGCCCAACCCTCAGCAGTTTCTTAAGACCCATCAGA |
| | | TGTTTCCAGGCTCCCCCAAGGACCTGAAATGACCCTGCGCCTTATT |
| | | TGAATTAACCAATCAGCCTGCTTCTCGCTTCTGTTCGCGCGCTTCT |
| | | GCTTCCCGAGCTCTATAAAAGAGCTCACAACCCCTCACTCGGCGCG |
| | | CCAGTCCTCCGACAGACTGAGTCGCCCGGGGG |
| linker/tag Nter | 36 | MDYKDDDDKGSSGTGSGSGTS |
| fusion | | |
| NS3 protease | 37 | APITAYAQQTRGLLGCIITSLTGRDKNQVEGEVQIVSTATQTFLAT |
| domain Nter | | CINGVCWAVYHGAGTRTIASPKGPVIQMYTNVDQDLVGWPAPQGSR |
| | | SLTPCTCGSSDLYLVTRHADVIPVRRRGDSRGSLLSPRPISYLKGS |
| | | SGGPLLCPAGHAVGLFRAAVCTRGVAKAVDFIPVENLETTMRSPVF |
| | | TD |
| NS3/NS4 degron | 38 | NSSPPAVTLTHPITKIDTKYIMTCMSADLEVVTSTWVLVGGVLAAL |
| Nter | | AAYCLSTGCVVIVGRIVLSGKPAGSSGSSIIPDREVLYQEF |
| NS3 protease | 39 | EDVVPCSMG |
| target site Nter | | |
| pCLS30018 | 40 | MDYKDDDDKGSSGTGSGSGTSAPITAYAQQTRGLLGCIITSLTGRD |
| | | KNQVEGEVQIVSTATQTFLATCINGVCWAVYHGAGTRTIASPKGPV |
| | | IQMYTNVDQDLVGWPAPQGSRSLTPCTCGSSDLYLVTRHADVIPVR |
| | | RRGDSRGSLLSPRPISYLKGSSGGPLLCPAGHAVGLFRAAVCTRGV |
| | | AKAVDFIPVENLETTMRSPVFTDNSSPPAVTLTHPITKIDTKYIMT |
| | | CMSADLEVVTSTWVLVGGVLAALAAYCLSTGCVVIVGRIVLSGKPA |
| | | GSSGSSIIPDREVLYQEFEDVVPCSMGSGAPMALPVTALLLPLALL |
| | | LHAARPQVQLQQSGPGLVKPSQTLSLTCAISGDSVSSNSAAWNWIR |
| | | QSPSRGLEWLGRTYYRSKWYNDYAVSVKSRITINPDTSKNQFSLQL |
| | | NSVTPEDTAVYYCAREVTGDLEDAFDIWGQGTMVTVSSGGGGSGGG |
| | | GSGGGGSDIQMTQSPSSLSASVGDRVTITCRASQTIWSYLNWYQQR |
| | | PGKAPNLLIYAASSLQSGVPSRFSGRGSGTDFTLTISSLQAEDFAT |
| | | YYCQQSYSIPQTFGQGTKLEIKAPTTTPAPRPPTPAPTIASQPLSL |
| | | RPEACRPAAGGAVHTRGLDFACDIYIWAPLAGTCGVLLLSLVITLY |
| | | CRRGRKKLLYIFKQPFMRPVQTTQEEDGCSCRFPEEEEGGCELRVK |
| | | FSRSADAPAYQQGQNQLYNELNLGRREEYDVLDKRRGRDPEMGGKP |
| | | RRKNPQEGLYNELQKDKMAEAYSEIGMKGERRRGKGHDGLYQGLST |
| | | ATKDTYDALHMQALPPR |
| IKB based degron | 41 | VNRVTYQGYSPYQLTWGRPSTRIQQQLGQLTLENLQML |
| pCLS30575 | 42 | ATGGCTCTGCCCGTCACCGCTCTGCTGCTGCCACTGGCACTGCTGC |
| (CD22-degron- | | TGCACGCTGCTAGGCCCCAGGTGCAGCTGCAGCAGAGCGGCCCTGG |
| IKB, part of NF- | | CCTGGTGAAGCCAAGCCAGACACTGTCCCTGACCTGCGCCATCAGC |
| kappa-B inhibitor | | GGCGATTCCGTGAGCTCCAACTCCGCCGCCTGGAATTGGATCAGGC |
| alpha, Gene: | | AGTCCCCTTCTCGGGGCCTGGAGTGGCTGGGAAGGACATACTATCG |
| NFKBIA) | | GTCTAAGTGGTACAACGATTATGCCGTGTCTGTGAAGAGCAGAATC |
| | | ACAATCAACCCTGACACCTCCAAGAATCAGTTCTCTCTGCAGCTGA |
| | | ATAGCGTGACACCAGAGGACACCGCCGTGTACTATTGCGCCAGGGA |
| | | GGTGACCGGCGACCTGGAGGATGCCTTTGACATCTGGGGCCAGGGC |
| | | ACAATGGTGACCGTGTCTAGCGGAGGCGGAGGCTCCGGAGGCGGAG |
| | | GATCTGGCGGAGGCGGAAGCGATATCCAGATGACACAGTCCCCATC |
| | | CTCTCTGAGCGCCTCCGTGGGCGACAGAGTGACAATCACCTGTAGG |
| | | GCCTCCCAGACCATCTGGTCTTACCTGAACTGGTATCAGCAGAGGC |
| | | CCGGCAAGGCCCCTAATCTGCTGATCTACGCAGCAAGCTCCCTGCA |
| | | GAGCGGAGTGCCATCCAGATTCTCTGGCAGGGGCTCCGGCACAGAC |
| | | TTCACCCTGACCATCTCTAGCCTCCAGGCCGAGGACTTCGCCACCT |
| | | ACTATTGCCAGCAGTCTTATAGCATCCCCCAGACATTTGGCCAGGG |
| | | CACCAAGCTGGAGATCAAGGCTCCCACCACAACCCCCGCTCCAAGG |
| | | CCCCCTACCCCCGCACCAACTATTGCCTCCCAGCCACTCTCACTGC |
| | | GGCCTGAGGCCTGTCGGCCCGCTGCTGGAGGCGCAGTGCATACAAG |
| | | GGGCCTCGATTTCGCCTGCGATATTTACATCTGGGCACCCCTCGCC |
| | | GGCACCTGCGGGGTGCTTCTCCTCTCCCTGGTGATTACCCTGTATT |
| | | GCAGACGGGGCCGGAAGAAGCTCCTCTACATTTTTAAGCAGCCTTT |
| | | CATGCGGCCAGTGCAGACAACCCAAGAGGAGGATGGGTGTTCCTGC |
| | | AGATTCCCTGAGGAAGAGGAAGGCGGGTGCGAGCTGAGAGTGAAGT |
| | | TCTCCAGGAGCGCAGATGCCCCCGCCTATCAACAGGGCCAGAACCA |
| | | GCTCTACAACGAGCTTAACCTCGGGAGGCGCGAAGAATACGACGTG |
| | | TTGGATAAGAGAAGGGGGCGGGACCCCGAGATGGGAGGAAAGCCCC |
| | | GGAGGAAGAACCCTCAGGAGGGCCTGTACAACGAGCTGCAGAAGGA |
| | | TAAGATGGCCGAGGCCTACTCAGAGATCGGGATGAAGGGGGAGCGG |
| | | CGCCGCGGGAAGGGGCACGATGGGCTCTACCAGGGGCTGAGCACAG |
| | | CCACAAAGGACACATACGACGCCTTGCACATGCAGGCCCTTCCACC |
| | | CCGGTCTGGAGATGAGATGGAAGAGTGCTCTCAGCACTTACCCGGC |
| | | GCCGGCAGTAGTGGCGATATCATGGATTACAAGGATGACGACGATA |
| | | AGGGCTCTTCCGGGACAGGCTCCGGATCCGGCACTAGTGCGCCCAT |
| | | CACGGCGTACGCCCAGCAGACGAGAGGCCTCCTAGGGTGTATAATC |
| | | ACCAGCCTGACTGGCCGGGACAAAAACCAAGTGGAGGGTGAGGTCC |
| | | AGATCGTGTCAACTGCTACCCAAACCTTCCTGGCAACGTGCATCAA |
| | | TGGGGTATGCTGGGCAGTCTACCACGGGGCCGGAACGAGGACCATC |
| | | GCATCACCCAAGGGTCCTGTCATCCAGATGTATACCAATGTGGACC |
| | | AAGACCTTGTGGGCTGGCCCGCTCCTCAAGGTTCCCGCTCATTGAC |
| | | ACCCTGTACCTGCGGCTCCTCGGACCTTTACCTGGTCACGAGGCAC |
| | | GCCGATGTCATTCCCGTGCGCCGGCGAGGTGATAGCAGGGGTAGCC |
| | | TGCTTTCGCCCCGGCCCATTTCCTACTTGAAAGGCTCCTCTGGGGG |
| | | TCCGCTGTTGTGCCCCGCGGGACACGCCGTGGGCCTATTCAGGGCC |
| | | GCGGTGTGCACCCGTGGAGTGGCTAAAGCGGTGGACTTTATCCCTG |
| | | TGGAGAACCTAGAGACAACCATGAGATCCCCGGTGTTCACGGACAA |
| | | CTCCTCTCCACCAGCAGTCACCCTGACGGTGAACAGGGTGACCTAC |
| | | CAGGGCTACAGCCCCTACCAGCTGACCTGGGGCAGGCCCAGCACCA |
| | | GGATCCAGCAGCAGCTGGGCCAGCTGACCCTGGAGAACCTGCAGAT |
| | | GCTG |
| SMNd7 based | 43 | YMSGYHTGYYMEMLA |
| degron (CD22- | | |
| degron-SMNd7, | | |
| part of SMN2 | | |
| lacking exon 7, | | |
| SMNDelta7 | | |
| pCLS30576 | 44 | ATGGCTCTGCCCGTCACCGCTCTGCTGCTGCCACTGGCACTGCTGC |
| (CD22-degron- | | TGCACGCTGCTAGGCCCCAGGTGCAGCTGCAGCAGAGCGGCCCTGG |
| SMNd7) | | CCTGGTGAAGCCAAGCCAGACACTGTCCCTGACCTGCGCCATCAGC |
| | | GGCGATTCCGTGAGCTCCAACTCCGCCGCCTGGAATTGGATCAGGC |
| | | AGTCCCCTTCTCGGGGCCTGGAGTGGCTGGGAAGGACATACTATCG |
| | | GTCTAAGTGGTACAACGATTATGCCGTGTCTGTGAAGAGCAGAATC |
| | | ACAATCAACCCTGACACCTCCAAGAATCAGTTCTCTCTGCAGCTGA |
| | | ATAGCGTGACACCAGAGGACACCGCCGTGTACTATTGCGCCAGGGA |
| | | GGTGACCGGCGACCTGGAGGATGCCTTTGACATCTGGGGCCAGGGC |
| | | ACAATGGTGACCGTGTCTAGCGGAGGCGGAGGCTCCGGAGGCGGAG |
| | | GATCTGGCGGAGGCGGAAGCGATATCCAGATGACACAGTCCCCATC |
| | | CTCTCTGAGCGCCTCCGTGGGCGACAGAGTGACAATCACCTGTAGG |
| | | GCCTCCCAGACCATCTGGTCTTACCTGAACTGGTATCAGCAGAGGC |
| | | CCGGCAAGGCCCCTAATCTGCTGATCTACGCAGCAAGCTCCCTGCA |
| | | GAGCGGAGTGCCATCCAGATTCTCTGGCAGGGGCTCCGGCACAGAC |
| | | TTCACCCTGACCATCTCTAGCCTCCAGGCCGAGGACTTCGCCACCT |
| | | ACTATTGCCAGCAGTCTTATAGCATCCCCCAGACATTTGGCCAGGG |
| | | CACCAAGCTGGAGATCAAGGCTCCCACCACAACCCCCGCTCCAAGG |
| | | CCCCCTACCCCCGCACCAACTATTGCCTCCCAGCCACTCTCACTGC |
| | | GGCCTGAGGCCTGTCGGCCCGCTGCTGGAGGCGCAGTGCATACAAG |
| | | GGGCCTCGATTTCGCCTGCGATATTTACATCTGGGCACCCCTCGCC |
| | | GGCACCTGCGGGGTGCTTCTCCTCTCCCTGGTGATTACCCTGTATT |
| | | GCAGACGGGGCCGGAAGAAGCTCCTCTACATTTTTAAGCAGCCTTT |
| | | CATGCGGCCAGTGCAGACAACCCAAGAGGAGGATGGGTGTTCCTGC |
| | | AGATTCCCTGAGGAAGAGGAAGGCGGGTGCGAGCTGAGAGTGAAGT |
| | | TCTCCAGGAGCGCAGATGCCCCCGCCTATCAACAGGGCCAGAACCA |
| | | GCTCTACAACGAGCTTAACCTCGGGAGGCGCGAAGAATACGACGTG |
| | | TTGGATAAGAGAAGGGGGCGGGACCCCGAGATGGGAGGAAAGCCCC |
| | | GGAGGAAGAACCCTCAGGAGGGCCTGTACAACGAGCTGCAGAAGGA |
| | | TAAGATGGCCGAGGCCTACTCAGAGATCGGGATGAAGGGGGAGCGG |
| | | CGCCGCGGGAAGGGGCACGATGGGCTCTACCAGGGGCTGAGCACAG |
| | | CCACAAAGGACACATACGACGCCTTGCACATGCAGGCCCTTCCACC |
| | | CCGGTCTGGAGATGAGATGGAAGAGTGCTCTCAGCACTTACCCGGC |
| | | GCCGGCAGTAGTGGCGATATCATGGATTACAAGGATGACGACGATA |
| | | AGGGCTCTTCCGGGACAGGCTCCGGATCCGGCACTAGTGCGCCCAT |
| | | CACGGCGTACGCCCAGCAGACGAGAGGCCTCCTAGGGTGTATAATC |
| | | ACCAGCCTGACTGGCCGGGACAAAAACCAAGTGGAGGGTGAGGTCC |
| | | AGATCGTGTCAACTGCTACCCAAACCTTCCTGGCAACGTGCATCAA |
| | | TGGGGTATGCTGGGCAGTCTACCACGGGGCCGGAACGAGGACCATC |
| | | GCATCACCCAAGGGTCCTGTCATCCAGATGTATACCAATGTGGACC |
| | | AAGACCTTGTGGGCTGGCCCGCTCCTCAAGGTTCCCGCTCATTGAC |
| | | ACCCTGTACCTGCGGCTCCTCGGACCTTTACCTGGTCACGAGGCAC |
| | | GCCGATGTCATTCCCGTGCGCCGGCGAGGTGATAGCAGGGGTAGCC |
| | | TGCTTTCGCCCCGGCCCATTTCCTACTTGAAAGGCTCCTCTGGGGG |
| | | TCCGCTGTTGTGCCCCGCGGGACACGCCGTGGGCCTATTCAGGGCC |
| | | GCGGTGTGCACCCGTGGAGTGGCTAAAGCGGTGGACTTTATCCCTG |
| | | TGGAGAACCTAGAGACAACCATGAGATCCCCGGTGTTCACGGACAA |
| | | CTCCTCTCCACCAGCAGTCACCCTGACGTACATGAGCGGCTACCAC |
| | | ACCGGCTACTACATGGAGATGCTGGCC |

### Example 2

Characterization of Surface Expression of C-Terminal Fusion CARs in Primary Human T-Cells

Peripheral blood mononuclear cells (PBMCs) were thawed and plated at 1×10⁶ cells/ml media in X-vivo-15 media (Lonza cat # BE04-418Q) supplemented with 5% AB serum (Seralab cat # GEM-100-318) and 20 ng/ml IL-2 (Miltenyi Biotech cat #130-097-748) for overnight culture at 37° C. PBMC were activated using human T activator CD3/CD28 (Life Technology cat #11132D) in X-vivo-15 media supplemented with 5% AB serum and 20 ng/ml IL-2.

1×10⁶ activated PBMCs (in 600 μl) were immediately incubated upon activation without removing the beads in an untreated 12 well plate pre-coated with 30 pg/mL retronectine (Takara cat # T100B) in the presence of the lentiviral particles prepared in Example 1 encoding the degron CARs for 2 h at 37° C. 600 μl of 2× X-vivo-15 media (X-vivo-15, 10% AB serum and 40 ng/ml IL-2) is then added and the cells were further incubated at 37° C. for 72 h. 3-5 days post transduction T-cells were incubated with or without 500 nM Asunaprevir for 48 h. The proportion of T-cells expressing the CAR at their surface was then quantified using labeled recombinant protein CD22 or CD123 targeted by the CAR (LakePharma).

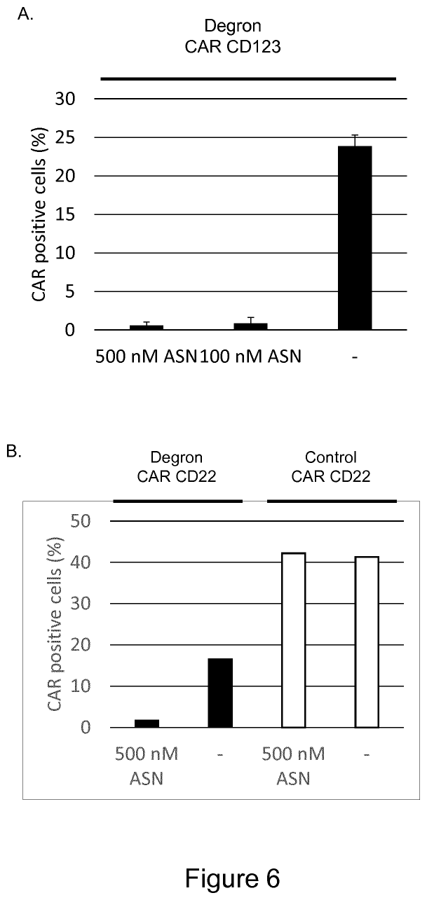

The results showed that CAR presentation at the surface of the transduced T-cells population could be controlled by Asunaprevir (FIG. 6), while control CARs lacking the self-excising degron did not react to Asunaprevir.

### Example 3

Characterization of Cytolytic Properties of C-Terminal Fusion Degron CARs in Primary Human T-Cells by Addition of Asunaprevir Protease Inhibitor

PBMCs are thawed and plated at 1×10⁶ cells/ml media in X-vivo-15 media (Lonza cat # BE04-418Q) supplemented with 5% AB serum (Seralab cat # GEM-100-318) and 20 ng/ml IL-2 (Miltenyi Biotech cat #130-097-748) for overnight culture at 37° C. PBMCs were activated using human T activator CD3/CD28 (Life Technology cat #11132D) in X-vivo-15 media supplemented with 5% AB serum and 20 ng/ml IL-2. 1×10⁶ activated PBMCs (in 600 μl) were immediately incubated upon activation without removing the beads in an untreated 12 well plate pre-coated with 30 pg/mL retronectine (Takara cat # T100B) in the presence of lentiviral particles encoding the engineered CARs of example 1 for 2 h at 37° C. 600 μl of 2× X-vivo-15 media (X-vivo-15, 10% AB serum and 40 ng/ml IL-2) was then added and the cells are incubated at 37° C. for 72 h. Transduced T-cells (1.5E6 cells) were incubated in complete X-vivo-15 media supplemented or not with 500 nM of Asunaprevir (Apexbio Technology or MedChem Express) in a 3:1 ratio with target cells presenting the CAR target antigen (Raji) and expressing a luciferase (0.5E6 cells) in a 12 wells plate. After 24 h the cells were pelleted, the supernatant was collected for luciferase quantification and the pelleted cells were resuspended in fresh complete X-vivo (supplemented or not with 500 nM Asunaprevir) media and 0.5×10⁶ target cells (CD22 positive cells) were added. This step was repeated for 3 consecutive days. The results showed that the CAR cytolytic properties into the transduced T-cells (killing of CD22 positive cells) were maintained and could be negatively controlled using the Asunaprevir (FIG. 6).

### Example 4

Characterization of Cytolytic Properties of C-Terminal Fusion Degron CARs in Primary Human T-Cells after Wash-Out of the Asunaprevir Protease Inhibitor

PBMC were transduced as described in example 3 with the engineered anti-CD22 degron CAR as described in example 3 and incubated in complete X-vivo-15 media supplemented or not with 500 nM of Asunaprevir (Apexbio Technology or MedChem Express). After 72 h a fraction of the cells incubated initially with 500 nM of Asunaprevir are washed and incubated at 37° C. in complete X-vivo-15 (X-vivo-15, 5% AB serum and 20 ng/ml IL-2) media (correspond to the wash-out 48 h prior to cytotoxicity assay point). After 96 h another fraction of the cells incubated initially with 500 nM of Asunaprevir is washed and incubated at 37° C. in complete X-vivo-15 media (correspond to the wash-out 24 h prior to cytotoxicity assay point). After 120 h another fraction of the cells incubated initially with 500 nM of Asunaprevir is washed and incubated at 37° C. in complete X-vivo-15 media (correspond to the wash-out at cytotoxicity assay point). A fraction of the cells is maintained under 500 nM of Asunaprevir (correspond to the no wash-out point).

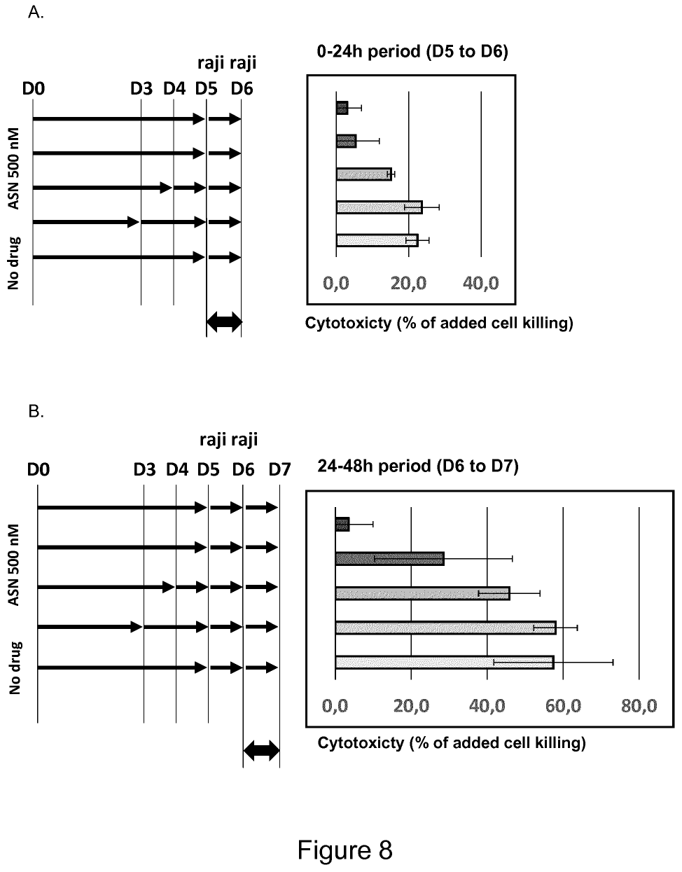

The different fractions of transduced T-cells are incubated in complete X-vivo-15 media supplemented (no-wash-out point) or not (all other points) with 500 nM of Asunaprevir (Apexbio Technology or MedChem Express) in a 3:1 ratio with target cells presenting the CAR target antigen (Raji) and expressing a luciferase in a 12 wells plate. After 24 h the cells are pelleted, the supernatant is collected for luciferase quantification. The results showed that the CAR cytolytic properties are controlled by Asunaprevir in a reversible manner (FIGS. 8A and 8B) since the CAR activity is increased when Asunaprevir gets progressively reduced.

### Example 5

### T-Cell Proliferation of the Asunaprevir (ASN) Protease Inhibitor

T-cells were cultured in X-Vivo 15 (Lonza) supplemented with 5% human serum hAB (Gemini) and 20 ng/ml IL-2 (Miltenyi) at a density of 1×10⁶ cells/ml in presence of various dose (0-1000 nM) of the Asunaprevir protease inhibitor.

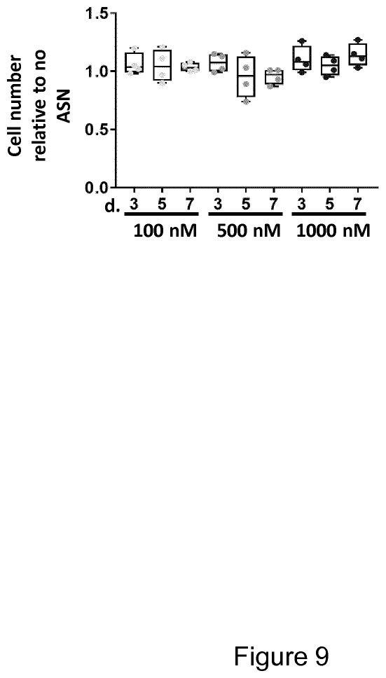

The results showed no effects of the small molecule ASN on the proliferation and viability of the T-cells after treatment with 100 nM to 1 μM ASN (FIG. 9).

### Example 6

### Cytokine Profiling in Presence of the Asunaprevir Protease Inhibitor

T-cells were co-cultured with Raji target cells in 12-well culture plates in the presence of various concentrations of ASN for 24 hours. Cells were spun down, and the supernatants were aliquoted and frozen. Cytokine levels in the supernatants were measured with LEGEND plex Human Th Cytokine panel (Biolegend).

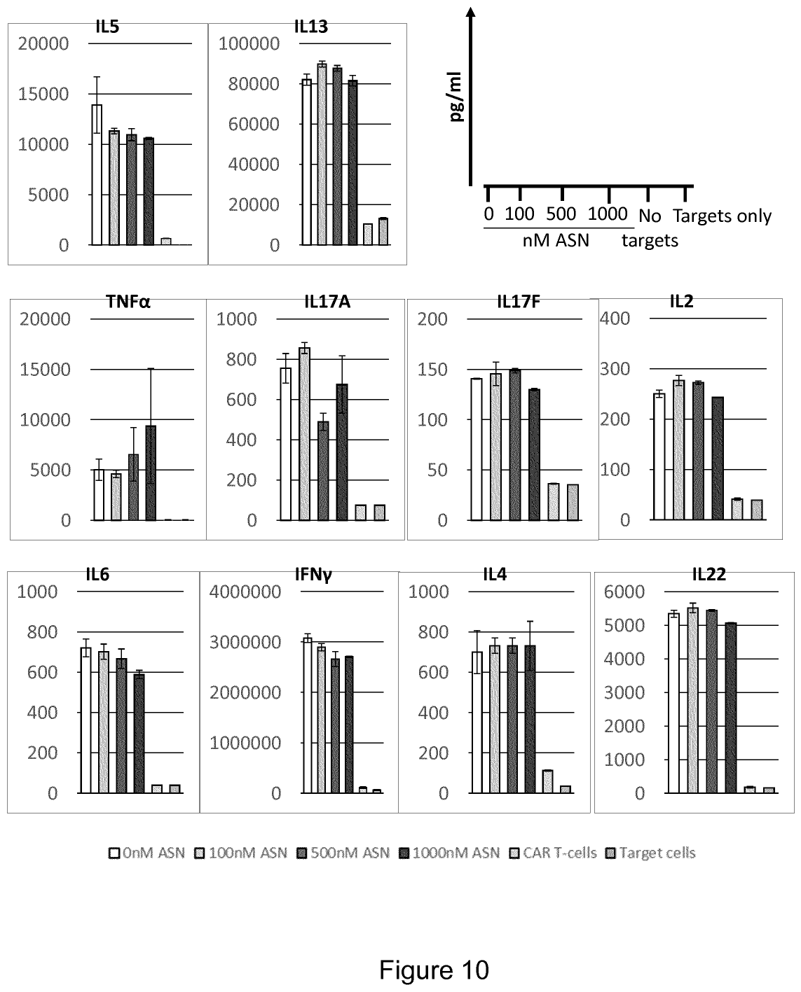

The results showed that the treatment with ASN did not result in notable variations (increases or decreases) in cytokine production (FIG. 10).

### Example 7

### Characterization of Surface Expression of C-Terminal Fusion CARs in Primary Human T-Cells

PBMCs are thawed and plated at 1×10⁶ cells/ml media in X-vivo-15 media (Lonza cat # BE04-418Q) supplemented with 5% AB serum (Seralab cat # GEM-100-318) and 20 ng/ml IL-2 (Miltenyi Biotech cat #130-097-748) for overnight culture at 37° C.

PBMCs are activated using human T activator CD3/CD28 (Life Technology cat #11132D) in X-vivo-15 media supplemented with 5% AB serum and 20 ng/ml IL-2. 1×10⁶ activated PBMCs (in 600 μl) are immediately incubated without removing the beads in an untreated 12 well plate pre-coated with 30 pg/mL retronectine (Takara cat # T100B) in the presence of increasing volume of lentiviral particles encoding the engineered SWOFF anti-CD22 CAR (SEQ ID NO:68) for 2 h at 37° C. 600 μl of 2× X-vivo-15 media (X-vivo-15, 10% AB serum and 40 ng/ml IL-2) is then added and the cells are incubated at 37° C. for 72 h. 3-5 days post transduction T-cells were incubated with or without 500 nM Asunaprevir for 48 h. The expression of the surface CAR (measured by mean fluorescence intensity (MFI)) were recorded using labeled recombinant protein (LakePharma).

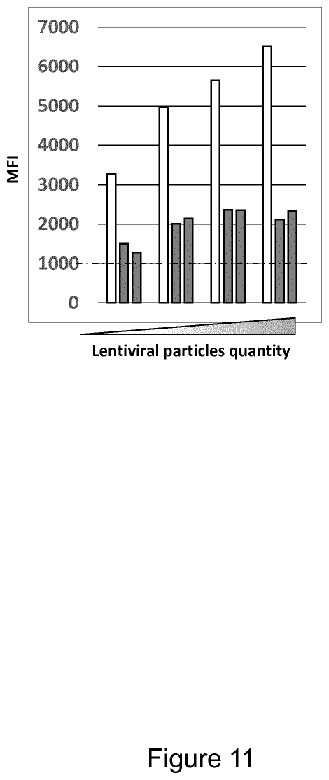

The results showed that the addition of ASN to the culture medium markedly decreased the MFI of the CAR-positive population (FIG. 11).

### Example 8

### Integration of the Engineered CAR at the TRAC Locus

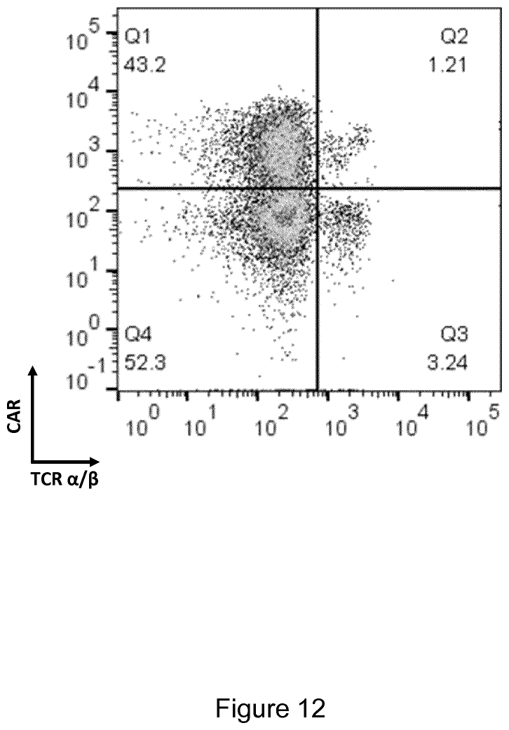

A repair matrix (SEQ ID NO:59) for homologous recombination encoding the TRAC left homology (SEQ ID NO:60) followed by a HA tag (SEQ ID NO:61), followed by 2A “self-cleaving” peptide (SEQ ID NO:62) that recovers the TCR reading frame followed by the SWOFF anti-CD22 CAR (SEQ ID NO:63) followed by BGH polyadenylation signal (SEQ ID NO:64) followed by the TRAC right homology (SEQ ID NO:65) was designed assembled and cloned in a vector allowing production of recombinant adeno-associated virus (rAAV6) according to standard molecular biology procedures (FIG. 12). The different sequences are reported in Table 11.

Human PBMCs were thawed and plated at 1×10⁶ cells/ml in X-vivo-15 media (Lonza) supplemented with 5% hAB serum (Gemini) or CTS Immune Cell SR (ThermoFisher) and 20 ng/ml IL-2 (Miltenyi Biotech) for overnight culture at 37° C. The following day the PBMCs were activated using human T activator CD3/CD28 (Life Technology) and cultured at a density of 1×10⁶ cells/ml for 3 days in X-vivo-15 media supplemented with 5% hAB serum or CTS Immune Cell SR and 20 ng/ml IL-2.

T-cells were then passaged the day prior to the transfection/transduction at 1×10⁶ cells/ml in complete media. On the day of transfection/transduction, the cells were de-beaded by magnetic separation (EasySep), washed twice in Cytoporation buffer T (BTX Harvard Apparatus, Holliston, Mass.), and resuspended at a final concentration of 28×10⁶ cells/ml in the same solution. The cell suspension was mixed with 2.5 μg mRNA encoding TALE-nuclease arms heterodimer polypeptides (SEQ ID NO:69 and SEQ ID NO:70 respectively) in a final volume of 200 μl. Transfection was performed using Pulse Agile technology, applying two 0.1 mS pulses at 3,000 V/cm followed by four 0.2 mS pulses at 325 V/cm in 0.4 cm gap cuvettes and in a final volume of 200 μl of Cytoporation buffer T (BTX Harvard Apparatus, Holliston, Mass.).

The electroporated cells were then immediately transferred to a 12-well plate containing 1 ml of prewarmed X-vivo-15 serum-free media and incubated for 37° C. for 15 min. The cells were then plated at a concentration of 10,000 cells/well with AAV in a 20 μl total volume of serum-free media (MOI: 1×10⁵ vg/cells) in 96-well round bottom plates. After 2 hours of culture at 30° C., 25 pL of Xvivo-15 media supplemented by 10% hAB serum and 40 ng/ml IL-2 was added to the cell suspension, and the mix was incubated 20 hours in the same culture conditions at 37° C. 100 pL of fresh complete media was then added. Six days after transduction, 0.5×10⁶ cells were seeded in a G-Rex 24-well plate (Wilson Wolf) in 5 ml of complete X-vivo-15 media and cultivated for 11 days.

Transduced T-cells (1.5×10⁶ cells) were incubated in X-vivo-15 media with 5% hAB serum, lacking II-2 supplemented with or without 1 to 500 nM Asunaprevir (Apexbio Technology or MedChem Express) in a 3:1 (T-cells: Targets) ratio with target cells (Raji) presenting the CAR target antigen and expressing a luciferase (0.5×10⁶ cells) in a 12-well plate. After 24 h, the cells are collected and mixed, and 100 ul of cells was used for luciferase quantification (OneGlo, Promega). The remainder of the cells were pelleted and resuspended in fresh X-vivo 15 media with 5% hAB serum, no 11-2 (supplemented with or without 1-500 nM Asunaprevir), and an additional 0.5×10⁶ target cells were added. This step was repeated for 3 consecutive days.

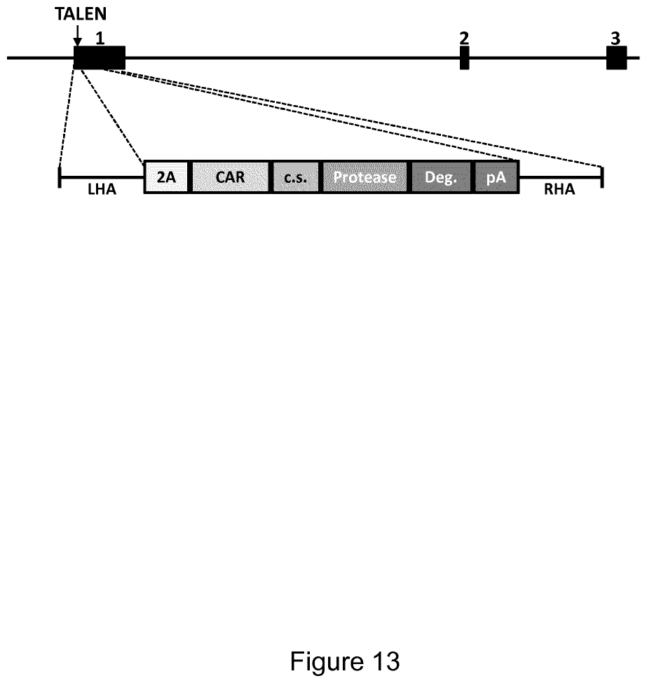

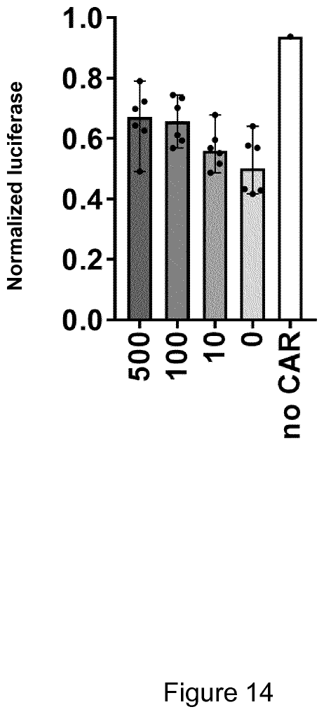

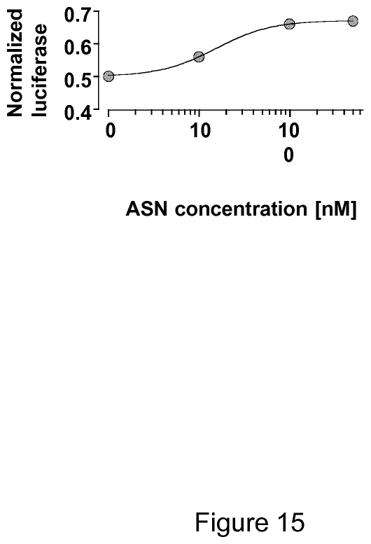

The results showed the efficient TRAC knock-out and CAR integration at the TRAC locus (FIG. 13). The results also showed that CAR T-cells cytolytic properties were controlled by addition of Asunaprevir (FIG. 14) with an IC50 of ˜15 mM (FIG. 15), within the range of concentrations that have been reported in the plasma of rodents, dogs and humans administered with ASN.

TABLE 11
polynucleotide and polypeptide sequences used in example 8

| SEQ ID NO: | Designation | Nucleotide/polypeptide sequence |
|---|---|---|
| 59 | integration | AAGTAGCCCTGCATTTCAGGTTTCCTTGAGTGGCAGGCCAGGCCTGGCCGTGA |
| | matrix | ACGTTCACTGAAATCATGGCCTCTTGGCCAAGATTGATAGCTTGTGCCTGTCC |
| | | CTGAGTCCCAGTCCATCACGAGCAGCTGGTTTCTAAGATGCTATTTCCCGTAT |
| | | AAAGCATGAGACCGTGACTTGCCAGCCCCACAGAGCCCCGCCCTTGTCCATCA |
| | | CTGGCATCTGGACTCCAGCCTGGGTTGGGGCAAAGAGGGAAATGAGATCATGT |
| | | CCTAACCCTGATCCTCTTGTCCCACAGATATCCAGTACCCCTACGACGTGCCC |
| | | GACTACGCCTCCGGTGAGGGCAGAGGAAGTCTTCTAACATGCGGTGACGTGGA |
| | | GGAGAATCCGGGCCCCGGATCCGCTCTGCCCGTCACCGCTCTGCTGCTGCCAC |
| | | TGGCACTGCTGCTGCACGCTGCTAGGCCCCAGGTGCAGCTGCAGCAGAGCGGC |
| | | CCTGGCCTGGTGAAGCCAAGCCAGACACTGTCCCTGACCTGCGCCATCAGCGG |
| | | CGATTCCGTGAGCTCCAACTCCGCCGCCTGGAATTGGATCAGGCAGTCCCCTT |
| | | CTCGGGGCCTGGAGTGGCTGGGAAGGACATACTATCGGTCTAAGTGGTACAAC |
| | | GATTATGCCGTGTCTGTGAAGAGCAGAATCACAATCAACCCTGACACCTCCAA |
| | | GAATCAGTTCTCTCTGCAGCTGAATAGCGTGACACCAGAGGACACCGCCGTGT |
| | | ACTATTGCGCCAGGGAGGTGACCGGCGACCTGGAGGATGCCTTTGACATCTGG |
| | | GGCCAGGGCACAATGGTGACCGTGTCTAGCGGAGGCGGAGGCTCCGGAGGCGG |
| | | AGGATCTGGCGGAGGCGGAAGCGATATCCAGATGACACAGTCCCCATCCTCTC |
| | | TGAGCGCCTCCGTGGGCGACAGAGTGACAATCACCTGTAGGGCCTCCCAGACC |
| | | ATCTGGTCTTACCTGAACTGGTATCAGCAGAGGCCCGGCAAGGCCCCTAATCT |
| | | GCTGATCTACGCAGCAAGCTCCCTGCAGAGCGGAGTGCCATCCAGATTCTCTG |
| | | GCAGGGGCTCCGGCACAGACTTCACCCTGACCATCTCTAGCCTCCAGGCCGAG |
| | | GACTTCGCCACCTACTATTGCCAGCAGTCTTATAGCATCCCCCAGACATTTGG |
| | | CCAGGGCACCAAGCTGGAGATCAAGGCTCCCACCACAACCCCCGCTCCAAGGC |
| | | CCCCTACCCCCGCACCAACTATTGCCTCCCAGCCACTCTCACTGCGGCCTGAG |
| | | GCCTGTCGGCCCGCTGCTGGAGGCGCAGTGCATACAAGGGGCCTCGATTTCGC |
| | | CTGCGATATTTACATCTGGGCACCCCTCGCCGGCACCTGCGGGGTGCTTCTCC |
| | | TCTCCCTGGTGATTACCCTGTATTGCAGACGGGGCCGGAAGAAGCTCCTCTAC |
| | | ATTTTTAAGCAGCCTTTCATGCGGCCAGTGCAGACAACCCAAGAGGAGGATGG |
| | | GTGTTCCTGCAGATTCCCTGAGGAAGAGGAAGGCGGGTGCGAGCTGAGAGTGA |
| | | AGTTCTCCAGGAGCGCAGATGCCCCCGCCTATCAACAGGGCCAGAACCAGCTC |
| | | TACAACGAGCTTAACCTCGGGAGGCGCGAAGAATACGACGTGTTGGATAAGAG |
| | | AAGGGGGCGGGACCCCGAGATGGGAGGAAAGCCCCGGAGGAAGAACCCTCAGG |
| | | AGGGCCTGTACAACGAGCTGCAGAAGGATAAGATGGCCGAGGCCTACTCAGAG |
| | | ATCGGGATGAAGGGGGAGCGGCGCCGCGGGAAGGGGCACGATGGGCTCTACCA |
| | | GGGGCTGAGCACAGCCACAAAGGACACATACGACGCCTTGCACATGCAGGCCC |
| | | TTCCACCCCGGTCTGGAGATGAGATGGAAGAGTGCTCTCAGCACTTACCCGGC |
| | | GCCGGCAGTAGTGGCGATATCATGGATTACAAGGATGACGACGATAAGGGCTC |
| | | TTCCGGGACAGGCTCCGGATCCGGCACTAGTGCGCCCATCACGGCGTACGCCC |
| | | AGCAGACGAGAGGCCTCCTAGGGTGTATAATCACCAGCCTGACTGGCCGGGAC |
| | | AAAAACCAAGTGGAGGGTGAGGTCCAGATCGTGTCAACTGCTACCCAAACCTT |
| | | CCTGGCAACGTGCATCAATGGGGTATGCTGGGCAGTCTACCACGGGGCCGGAA |
| | | CGAGGACCATCGCATCACCCAAGGGTCCTGTCATCCAGATGTATACCAATGTG |
| | | GACCAAGACCTTGTGGGCTGGCCCGCTCCTCAAGGTTCCCGCTCATTGACACC |
| | | CTGTACCTGCGGCTCCTCGGACCTTTACCTGGTCACGAGGCACGCCGATGTCA |
| | | TTCCCGTGCGCCGGCGAGGTGATAGCAGGGGTAGCCTGCTTTCGCCCCGGCCC |
| | | ATTTCCTACTTGAAAGGCTCCTCTGGGGGTCCGCTGTTGTGCCCCGCGGGACA |
| | | CGCCGTGGGCCTATTCAGGGCCGCGGTGTGCACCCGTGGAGTGGCTAAAGCGG |
| | | TGGACTTTATCCCTGTGGAGAACCTAGAGACAACCATGAGATCCCCGGTGTTC |
| | | ACGGACAACTCCTCTCCACCAGCAGTCACCCTGACGCACCCAATCACCAAAAT |
| | | CGATACCAAATACATCATGACATGCATGTCGGCCGACCTGGAGGTCGTCACGA |
| | | GCACCTGGGTGCTCGTTGGCGGCGTCCTGGCTGCTCTGGCCGCGTATTGCCTG |
| | | TCAACAGGCTGCGTGGTCATAGTGGGCAGGATCGTCTTGTCCGGGAAGCCGGC |
| | | AATTATACCTGACAGGGAGGTTCTCTACTGATCTAGAGGGCCCGTTTAAACCC |
| | | GCTGATCAGCCTCGACTGTGCCTTCTAGTTGCCAGCCATCTGTTGTTTGCCCC |
| | | TCCCCCGTGCCTTCCTTGACCCTGGAAGGTGCCACTCCCACTGTCCTTTCCTA |
| | | ATAAAATGAGGAAATTGCATCGCATTGTCTGAGTAGGTGTCATTCTATTCTGG |
| | | GGGGTGGGGTGGGGCAGGACAGCAAGGGGGAGGATTGGGAAGACAATAGCAGG |
| | | CATGCTGGGGATGCGGTGGGCTCTATGACTAGTGGCGAATTCCCGTGTACCAG |
| | | CTGAGAGACTCTAAATCCAGTGACAAGTCTGTCTGCCTATTCACCGATTTTGA |
| | | TTCTCAAACAAATGTGTCACAAAGTAAGGATTCTGATGTGTATATCACAGACA |
| | | AAACTGTGCTAGACATGAGGTCTATGGACTTCAAGAGCAACAGTGCTGTGGCC |
| | | TGGAGCAACAAATCTGACTTTGCATGTGCAAACGCCTTCAACAACAGCATTAT |
| | | TCCAGAAGACACCTTCTTCCCCAGCCCAGGTAAGGGCAGCTTTGGTGCCTTCG |
| | | CAGGCTGTTTCCTTGCTTCAGGAA |
| 60 | TRAC left | AAGTAGCCCTGCATTTCAGGTTTCCTTGAGTGGCAGGCCAGGCCTGGCCGTGA |
| | homology | ACGTTCACTGAAATCATGGCCTCTTGGCCAAGATTGATAGCTTGTGCCTGTCC |
| | | CTGAGTCCCAGTCCATCACGAGCAGCTGGTTTCTAAGATGCTATTTCCCGTAT |
| | | AAAGCATGAGACCGTGACTTGCCAGCCCCACAGAGCCCCGCCCTTGTCCATCA |
| | | CTGGCATCTGGACTCCAGCCTGGGTTGGGGCAAAGAGGGAAATGAGATCATGT |
| | | CCTAACCCTGATCCTCTTGTCCCACAGATATCCAG |
| 61 | HA tag | TACCCCTACGACGTGCCCGACTACGCC |
| 62 | 2A element | GAGGGCAGAGGAAGTCTTCTAACATGCGGTGACGTGGAGGAGAATCCGGGCCC |
| | | C |
| 63 | SWOFF | GCTCTGCCCGTCACCGCTCTGCTGCTGCCACTGGCACTGCTGCTGCACGCTGC |
| | anti CD22 | TAGGCCCCAGGTGCAGCTGCAGCAGAGCGGCCCTGGCCTGGTGAAGCCAAGCC |
| | CAR | AGACACTGTCCCTGACCTGCGCCATCAGCGGCGATTCCGTGAGCTCCAACTCC |
| | | GCCGCCTGGAATTGGATCAGGCAGTCCCCTTCTCGGGGCCTGGAGTGGCTGGG |
| | | AAGGACATACTATCGGTCTAAGTGGTACAACGATTATGCCGTGTCTGTGAAGA |
| | | GCAGAATCACAATCAACCCTGACACCTCCAAGAATCAGTTCTCTCTGCAGCTG |
| | | AATAGCGTGACACCAGAGGACACCGCCGTGTACTATTGCGCCAGGGAGGTGAC |
| | | CGGCGACCTGGAGGATGCCTTTGACATCTGGGGCCAGGGCACAATGGTGACCG |
| | | TGTCTAGCGGAGGCGGAGGCTCCGGAGGCGGAGGATCTGGCGGAGGCGGAAGC |
| | | GATATCCAGATGACACAGTCCCCATCCTCTCTGAGCGCCTCCGTGGGCGACAG |
| | | AGTGACAATCACCTGTAGGGCCTCCCAGACCATCTGGTCTTACCTGAACTGGT |
| | | ATCAGCAGAGGCCCGGCAAGGCCCCTAATCTGCTGATCTACGCAGCAAGCTCC |
| | | CTGCAGAGCGGAGTGCCATCCAGATTCTCTGGCAGGGGCTCCGGCACAGACTT |
| | | CACCCTGACCATCTCTAGCCTCCAGGCCGAGGACTTCGCCACCTACTATTGCC |
| | | AGCAGTCTTATAGCATCCCCCAGACATTTGGCCAGGGCACCAAGCTGGAGATC |
| | | AAGGCTCCCACCACAACCCCCGCTCCAAGGCCCCCTACCCCCGCACCAACTAT |
| | | TGCCTCCCAGCCACTCTCACTGCGGCCTGAGGCCTGTCGGCCCGCTGCTGGAG |
| | | GCGCAGTGCATACAAGGGGCCTCGATTTCGCCTGCGATATTTACATCTGGGCA |
| | | CCCCTCGCCGGCACCTGCGGGGTGCTTCTCCTCTCCCTGGTGATTACCCTGTA |
| | | TTGCAGACGGGGCCGGAAGAAGCTCCTCTACATTTTTAAGCAGCCTTTCATGC |
| | | GGCCAGTGCAGACAACCCAAGAGGAGGATGGGTGTTCCTGCAGATTCCCTGAG |
| | | GAAGAGGAAGGCGGGTGCGAGCTGAGAGTGAAGTTCTCCAGGAGCGCAGATGC |
| | | CCCCGCCTATCAACAGGGCCAGAACCAGCTCTACAACGAGCTTAACCTCGGGA |
| | | GGCGCGAAGAATACGACGTGTTGGATAAGAGAAGGGGGCGGGACCCCGAGATG |
| | | GGAGGAAAGCCCCGGAGGAAGAACCCTCAGGAGGGCCTGTACAACGAGCTGCA |
| | | GAAGGATAAGATGGCCGAGGCCTACTCAGAGATCGGGATGAAGGGGGAGCGGC |
| | | GCCGCGGGAAGGGGCACGATGGGCTCTACCAGGGGCTGAGCACAGCCACAAAG |
| | | GACACATACGACGCCTTGCACATGCAGGCCCTTCCACCCCGGTCTGGAGATGA |
| | | GATGGAAGAGTGCTCTCAGCACTTACCCGGCGCCGGCAGTAGTGGCGATATCA |
| | | TGGATTACAAGGATGACGACGATAAGGGCTCTTCCGGGACAGGCTCCGGATCC |
| | | GGCACTAGTGCGCCCATCACGGCGTACGCCCAGCAGACGAGAGGCCTCCTAGG |
| | | GTGTATAATCACCAGCCTGACTGGCCGGGACAAAAACCAAGTGGAGGGTGAGG |
| | | TCCAGATCGTGTCAACTGCTACCCAAACCTTCCTGGCAACGTGCATCAATGGG |
| | | GTATGCTGGGCAGTCTACCACGGGGCCGGAACGAGGACCATCGCATCACCCAA |
| | | GGGTCCTGTCATCCAGATGTATACCAATGTGGACCAAGACCTTGTGGGCTGGC |
| | | CCGCTCCTCAAGGTTCCCGCTCATTGACACCCTGTACCTGCGGCTCCTCGGAC |
| | | CTTTACCTGGTCACGAGGCACGCCGATGTCATTCCCGTGCGCCGGCGAGGTGA |
| | | TAGCAGGGGTAGCCTGCTTTCGCCCCGGCCCATTTCCTACTTGAAAGGCTCCT |
| | | CTGGGGGTCCGCTGTTGTGCCCCGCGGGACACGCCGTGGGCCTATTCAGGGCC |
| | | GCGGTGTGCACCCGTGGAGTGGCTAAAGCGGTGGACTTTATCCCTGTGGAGAA |
| | | CCTAGAGACAACCATGAGATCCCCGGTGTTCACGGACAACTCCTCTCCACCAG |
| | | CAGTCACCCTGACGCACCCAATCACCAAAATCGATACCAAATACATCATGACA |
| | | TGCATGTCGGCCGACCTGGAGGTCGTCACGAGCACCTGGGTGCTCGTTGGCGG |
| | | CGTCCTGGCTGCTCTGGCCGCGTATTGCCTGTCAACAGGCTGCGTGGTCATAG |
| | | TGGGCAGGATCGTCTTGTCCGGGAAGCCGGCAATTATACCTGACAGGGAGGTT |
| | | CTCTACTGA |
| 64 | BGH poly A | CGACTGTGCCTTCTAGTTGCCAGCCATCTGTTGTTTGCCCCTCCCCCGTGCCT |
| | | TCCTTGACCCTGGAAGGTGCCACTCCCACTGTCCTTTCCTAATAAAATGAGGA |
| | | AATTGCATCGCATTGTCTGAGTAGGTGTCATTCTATTCTGGGGGGTGGGGTGG |
| | | GGCAGGACAGCAAGGGGGAGGATTGGGAAGACAATAGCAGGCATGCTGGGGAT |
| | | GCGGTGGGCTCTATGA |
| 65 | TRAC light | GCTGAGAGACTCTAAATCCAGTGACAAGTCTGTCTGCCTATTCACCGATTTTG |
| | homology | ATTCTCAAACAAATGTGTCACAAAGTAAGGATTCTGATGTGTATATCACAGAC |
| | | AAAACTGTGCTAGACATGAGGTCTATGGACTTCAAGAGCAACAGTGCTGTGGC |
| | | CTGGAGCAACAAATCTGACTTTGCATGTGCAAACGCCTTCAACAACAGCATTA |
| | | TTCCAGAAGACACCTTCTTCCCCAGCCCAGGTAAGGGCAGCTTTGGTGCCTTC |
| | | GCAGGCTGTTTCCTTGCTTCAGGAA |
| 66 | TRAC | ATGGGCGATCCTAAAAAGAAACGTAAGGTCATCGATATCGCCGATCTACGCAC |
| | TALEN left | GCTCGGCTACAGCCAGCAGCAACAGGAGAAGATCAAACCGAAGGTTCGTTCGA |
| | | CAGTGGCGCAGCACCACGAGGCACTGGTCGGCCACGGGTTTACACACGCGCAC |
| | | ATCGTTGCGTTAAGCCAACACCCGGCAGCGTTAGGGACCGTCGCTGTCAAGTA |
| | | TCAGGACATGATCGCAGCGTTGCCAGAGGCGACACACGAAGCGATCGTTGGCG |
| | | TCGGCAAACAGTGGTCCGGCGCACGCGCTCTGGAGGCCTTGCTCACGGTGGCG |
| | | GGAGAGTTGAGAGGTCCACCGTTACAGTTGGACACAGGCCAACTTCTCAAGAT |
| | | TGCAAAACGTGGCGGCGTGACCGCAGTGGAGGCAGTGCATGCATGGCGCAATG |
| | | CACTGACGGGTGCCCCGCTCAACTTGACCCCCCAGCAGGTGGTGGCCATCGCC |
| | | AGCAATGGCGGTGGCAAGCAGGCGCTGGAGACGGTCCAGCGGCTGTTGCCGGT |
| | | GCTGTGCCAGGCCCACGGCTTGACCCCCCAGCAGGTGGTGGCCATCGCCAGCA |
| | | ATAATGGTGGCAAGCAGGCGCTGGAGACGGTCCAGCGGCTGTTGCCGGTGCTG |
| | | TGCCAGGCCCACGGCTTGACCCCCCAGCAGGTGGTGGCCATCGCCAGCAATGG |
| | | CGGTGGCAAGCAGGCGCTGGAGACGGTCCAGCGGCTGTTGCCGGTGCTGTGCC |
| | | AGGCCCACGGCTTGACCCCGGAGCAGGTGGTGGCCATCGCCAGCCACGATGGC |
| | | GGCAAGCAGGCGCTGGAGACGGTCCAGCGGCTGTTGCCGGTGCTGTGCCAGGC |
| | | CCACGGCTTGACCCCGGAGCAGGTGGTGGCCATCGCCAGCCACGATGGCGGCA |
| | | AGCAGGCGCTGGAGACGGTCCAGCGGCTGTTGCCGGTGCTGTGCCAGGCCCAC |
| | | GGCTTGACCCCGGAGCAGGTGGTGGCCATCGCCAGCCACGATGGCGGCAAGCA |
| | | GGCGCTGGAGACGGTCCAGCGGCTGTTGCCGGTGCTGTGCCAGGCCCACGGCT |
| | | TGACCCCGGAGCAGGTGGTGGCCATCGCCAGCAATATTGGTGGCAAGCAGGCG |
| | | CTGGAGACGGTGCAGGCGCTGTTGCCGGTGCTGTGCCAGGCCCACGGCTTGAC |
| | | CCCGGAGCAGGTGGTGGCCATCGCCAGCCACGATGGCGGCAAGCAGGCGCTGG |
| | | AGACGGTCCAGCGGCTGTTGCCGGTGCTGTGCCAGGCCCACGGCTTGACCCCG |
| | | GAGCAGGTGGTGGCCATCGCCAGCAATATTGGTGGCAAGCAGGCGCTGGAGAC |
| | | GGTGCAGGCGCTGTTGCCGGTGCTGTGCCAGGCCCACGGCTTGACCCCCCAGC |
| | | AGGTGGTGGCCATCGCCAGCAATAATGGTGGCAAGCAGGCGCTGGAGACGGTC |
| | | CAGCGGCTGTTGCCGGTGCTGTGCCAGGCCCACGGCTTGACCCCGGAGCAGGT |
| | | GGTGGCCATCGCCAGCAATATTGGTGGCAAGCAGGCGCTGGAGACGGTGCAGG |
| | | CGCTGTTGCCGGTGCTGTGCCAGGCCCACGGCTTGACCCCCCAGCAGGTGGTG |
| | | GCCATCGCCAGCAATGGCGGTGGCAAGCAGGCGCTGGAGACGGTCCAGCGGCT |
| | | GTTGCCGGTGCTGTGCCAGGCCCACGGCTTGACCCCGGAGCAGGTGGTGGCCA |
| | | TCGCCAGCAATATTGGTGGCAAGCAGGCGCTGGAGACGGTGCAGGCGCTGTTG |
| | | CCGGTGCTGTGCCAGGCCCACGGCTTGACCCCCCAGCAGGTGGTGGCCATCGC |
| | | CAGCAATGGCGGTGGCAAGCAGGCGCTGGAGACGGTCCAGCGGCTGTTGCCGG |
| | | TGCTGTGCCAGGCCCACGGCTTGACCCCGGAGCAGGTGGTGGCCATCGCCAGC |
| | | CACGATGGCGGCAAGCAGGCGCTGGAGACGGTCCAGCGGCTGTTGCCGGTGCT |
| | | GTGCCAGGCCCACGGCTTGACCCCTCAGCAGGTGGTGGCCATCGCCAGCAATG |
| | | GCGGCGGCAGGCCGGCGCTGGAGAGCATTGTTGCCCAGTTATCTCGCCCTGAT |
| | | CCGGCGTTGGCCGCGTTGACCAACGACCACCTCGTCGCCTTGGCCTGCCTCGG |
| | | CGGGCGTCCTGCGCTGGATGCAGTGAAAAAGGGATTGGGGGATCCTATCAGCC |
| | | GTTCCCAGCTGGTGAAGTCCGAGCTGGAGGAGAAGAAATCCGAGTTGAGGCAC |
| | | AAGCTGAAGTACGTGCCCCACGAGTACATCGAGCTGATCGAGATCGCCCGGAA |
| | | CAGCACCCAGGACCGTATCCTGGAGATGAAGGTGATGGAGTTCTTCATGAAGG |
| | | TGTACGGCTACAGGGGCAAGCACCTGGGCGGCTCCAGGAAGCCCGACGGCGCC |
| | | ATCTACACCGTGGGCTCCCCCATCGACTACGGCGTGATCGTGGACACCAAGGC |
| | | CTACTCCGGCGGCTACAACCTGCCCATCGGCCAGGCCGACGAAATGCAGAGGT |
| | | ACGTGGAGGAGAACCAGACCAGGAACAAGCACATCAACCCCAACGAGTGGTGG |
| | | AAGGTGTACCCCTCCAGCGTGACCGAGTTCAAGTTCCTGTTCGTGTCCGGCCA |
| | | CTTCAAGGGCAACTACAAGGCCCAGCTGACCAGGCTGAACCACATCACCAACT |
| | | GCAACGGCGCCGTGCTGTCCGTGGAGGAGCTCCTGATCGGCGGCGAGATGATC |
| | | AAGGCCGGCACCCTGACCCTGGAGGAGGTGAGGAGGAAGTTCAACAACGGCGA |
| | | GATCAACTTCGCGGCCGACTGATAA |
| 67 | TRAC | ATGGGCGATCCTAAAAAGAAACGTAAGGTCATCGATATCGCCGATCTACGCAC |
| | TALEN light | GCTCGGCTACAGCCAGCAGCAACAGGAGAAGATCAAACCGAAGGTTCGTTCGA |
| | | CAGTGGCGCAGCACCACGAGGCACTGGTCGGCCACGGGTTTACACACGCGCAC |
| | | ATCGTTGCGTTAAGCCAACACCCGGCAGCGTTAGGGACCGTCGCTGTCAAGTA |
| | | TCAGGACATGATCGCAGCGTTGCCAGAGGCGACACACGAAGCGATCGTTGGCG |
| | | TCGGCAAACAGTGGTCCGGCGCACGCGCTCTGGAGGCCTTGCTCACGGTGGCG |
| | | GGAGAGTTGAGAGGTCCACCGTTACAGTTGGACACAGGCCAACTTCTCAAGAT |
| | | TGCAAAACGTGGCGGCGTGACCGCAGTGGAGGCAGTGCATGCATGGCGCAATG |
| | | CACTGACGGGTGCCCCGCTCAACTTGACCCCGGAGCAGGTGGTGGCCATCGCC |
| | | AGCCACGATGGCGGCAAGCAGGCGCTGGAGACGGTCCAGCGGCTGTTGCCGGT |
| | | GCTGTGCCAGGCCCACGGCTTGACCCCCCAGCAGGTGGTGGCCATCGCCAGCA |
| | | ATGGCGGTGGCAAGCAGGCGCTGGAGACGGTCCAGCGGCTGTTGCCGGTGCTG |
| | | TGCCAGGCCCACGGCTTGACCCCGGAGCAGGTGGTGGCCATCGCCAGCCACGA |
| | | TGGCGGCAAGCAGGCGCTGGAGACGGTCCAGCGGCTGTTGCCGGTGCTGTGCC |
| | | AGGCCCACGGCTTGACCCCGGAGCAGGTGGTGGCCATCGCCAGCAATATTGGT |
| | | GGCAAGCAGGCGCTGGAGACGGTGCAGGCGCTGTTGCCGGTGCTGTGCCAGGC |
| | | CCACGGCTTGACCCCCCAGCAGGTGGTGGCCATCGCCAGCAATAATGGTGGCA |
| | | AGCAGGCGCTGGAGACGGTCCAGCGGCTGTTGCCGGTGCTGTGCCAGGCCCAC |
| | | GGCTTGACCCCGGAGCAGGTGGTGGCCATCGCCAGCCACGATGGCGGCAAGCA |
| | | GGCGCTGGAGACGGTCCAGCGGCTGTTGCCGGTGCTGTGCCAGGCCCACGGCT |
| | | TGACCCCCCAGCAGGTGGTGGCCATCGCCAGCAATGGCGGTGGCAAGCAGGCG |
| | | CTGGAGACGGTCCAGCGGCTGTTGCCGGTGCTGTGCCAGGCCCACGGCTTGAC |
| | | CCCCCAGCAGGTGGTGGCCATCGCCAGCAATAATGGTGGCAAGCAGGCGCTGG |
| | | AGACGGTCCAGCGGCTGTTGCCGGTGCTGTGCCAGGCCCACGGCTTGACCCCC |
| | | CAGCAGGTGGTGGCCATCGCCAGCAATAATGGTGGCAAGCAGGCGCTGGAGAC |
| | | GGTCCAGCGGCTGTTGCCGGTGCTGTGCCAGGCCCACGGCTTGACCCCCCAGC |
| | | AGGTGGTGGCCATCGCCAGCAATGGCGGTGGCAAGCAGGCGCTGGAGACGGTC |
| | | CAGCGGCTGTTGCCGGTGCTGTGCCAGGCCCACGGCTTGACCCCGGAGCAGGT |
| | | GGTGGCCATCGCCAGCAATATTGGTGGCAAGCAGGCGCTGGAGACGGTGCAGG |
| | | CGCTGTTGCCGGTGCTGTGCCAGGCCCACGGCTTGACCCCGGAGCAGGTGGTG |
| | | GCCATCGCCAGCCACGATGGCGGCAAGCAGGCGCTGGAGACGGTCCAGCGGCT |
| | | GTTGCCGGTGCTGTGCCAGGCCCACGGCTTGACCCCGGAGCAGGTGGTGGCCA |
| | | TCGCCAGCAATATTGGTGGCAAGCAGGCGCTGGAGACGGTGCAGGCGCTGTTG |
| | | CCGGTGCTGTGCCAGGCCCACGGCTTGACCCCGGAGCAGGTGGTGGCCATCGC |
| | | CAGCCACGATGGCGGCAAGCAGGCGCTGGAGACGGTCCAGCGGCTGTTGCCGG |
| | | TGCTGTGCCAGGCCCACGGCTTGACCCCCCAGCAGGTGGTGGCCATCGCCAGC |
| | | AATAATGGTGGCAAGCAGGCGCTGGAGACGGTCCAGCGGCTGTTGCCGGTGCT |
| | | GTGCCAGGCCCACGGCTTGACCCCTCAGCAGGTGGTGGCCATCGCCAGCAATG |
| | | GCGGCGGCAGGCCGGCGCTGGAGAGCATTGTTGCCCAGTTATCTCGCCCTGAT |
| | | CCGGCGTTGGCCGCGTTGACCAACGACCACCTCGTCGCCTTGGCCTGCCTCGG |
| | | CGGGCGTCCTGCGCTGGATGCAGTGAAAAAGGGATTGGGGGATCCTATCAGCC |
| | | GTTCCCAGCTGGTGAAGTCCGAGCTGGAGGAGAAGAAATCCGAGTTGAGGCAC |
| | | AAGCTGAAGTACGTGCCCCACGAGTACATCGAGCTGATCGAGATCGCCCGGAA |
| | | CAGCACCCAGGACCGTATCCTGGAGATGAAGGTGATGGAGTTCTTCATGAAGG |
| | | TGTACGGCTACAGGGGCAAGCACCTGGGCGGCTCCAGGAAGCCCGACGGCGCC |
| | | ATCTACACCGTGGGCTCCCCCATCGACTACGGCGTGATCGTGGACACCAAGGC |
| | | CTACTCCGGCGGCTACAACCTGCCCATCGGCCAGGCCGACGAAATGCAGAGGT |
| | | ACGTGGAGGAGAACCAGACCAGGAACAAGCACATCAACCCCAACGAGTGGTGG |
| | | AAGGTGTACCCCTCCAGCGTGACCGAGTTCAAGTTCCTGTTCGTGTCCGGCCA |
| | | CTTCAAGGGCAACTACAAGGCCCAGCTGACCAGGCTGAACCACATCACCAACT |
| | | GCAACGGCGCCGTGCTGTCCGTGGAGGAGCTCCTGATCGGCGGCGAGATGATC |
| | | AAGGCCGGCACCCTGACCCTGGAGGAGGTGAGGAGGAAGTTCAACAACGGCGA |
| | | GATCAACTTCGCGGCCG |
| 68 | SWOFF | ALPVTALLLPLALLLHAARPQVQLQQSGPGLVKPSQTLSLTCAISGDSVSSNS |
| | anti CD22 | AAWNWIRQSPSRGLEWLGRTYYRSKWYNDYAVSVKSRITINPDTSKNQFSLQL |
| | CAR | NSVTPEDTAVYYCAREVTGDLEDAFDIWGQGTMVTVSSGGGGSGGGGSGGGGS |
| | polypeptide | DIQMTQSPSSLSASVGDRVTITCRASQTIWSYLNWYQQRPGKAPNLLIYAASS |
| | | LQSGVPSRFSGRGSGTDFTLTISSLQAEDFATYYCQQSYSIPQTFGQGTKLEI |
| | | KAPTTTPAPRPPTPAPTIASQPLSLRPEACRPAAGGAVHTRGLDFACDIYIWA |
| | | PLAGTCGVLLLSLVITLYCRRGRKKLLYIFKQPFMRPVQTTQEEDGCSCRFPE |
| | | EEEGGCELRVKFSRSADAPAYQQGQNQLYNELNLGRREEYDVLDKRRGRDPEM |
| | | GGKPRRKNPQEGLYNELQKDKMAEAYSEIGMKGERRRGKGHDGLYQGLSTATK |
| | | DTYDALHMQALPPRSGDEMEECSQHLPGAGSSGDIMDYKDDDDKGSSGTGSGS |
| | | GTSAPITAYAQQTRGLLGCIITSLTGRDKNQVEGEVQIVSTATQTFLATCING |
| | | VCWAVYHGAGTRTIASPKGPVIQMYTNVDQDLVGWPAPQGSRSLTPCTCGSSD |
| | | LYLVTRHADVIPVRRRGDSRGSLLSPRPISYLKGSSGGPLLCPAGHAVGLFRA |
| | | AVCTRGVAKAVDFIPVENLETTMRSPVFTDNSSPPAVTLTHPITKIDTKYIMT |
| | | CMSADLEVVTSTWVLVGGVLAALAAYCLSTGCVVIVGRIVLSGKPAIIPDREV |
| | | LY |
| 69 | TRAC | MGDPKKKRKVIDIADLRTLGYSQQQQEKIKPKVRSTVAQHHEALVGHGFTHAH |
| | TALEN left | IVALSQHPAALGTVAVKYQDMIAALPEATHEAIVGVGKQWSGARALEALLTVA |
| | polypeptide | GELRGPPLQLDTGQLLKIAKRGGVTAVEAVHAWRNALTGAPLNLTPQQVVAIA |
| | | SNGGGKQALETVQRLLPVLCQAHGLTPQQVVAIASNNGGKQALETVQRLLPVL |
| | | CQAHGLTPQQVVAIASNGGGKQALETVQRLLPVLCQAHGLTPEQVVAIASHDG |
| | | GKQALETVQRLLPVLCQAHGLTPEQVVAIASHDGGKQALETVQRLLPVLCQAH |
| | | GLTPEQVVAIASHDGGKQALETVQRLLPVLCQAHGLTPEQVVAIASNIGGKQA |
| | | LETVQALLPVLCQAHGLTPEQVVAIASHDGGKQALETVQRLLPVLCQAHGLTP |
| | | EQVVAIASNIGGKQALETVQALLPVLCQAHGLTPQQVVAIASNNGGKQALETV |
| | | QRLLPVLCQAHGLTPEQVVAIASNIGGKQALETVQALLPVLCQAHGLTPQQVV |
| | | AIASNGGGKQALETVQRLLPVLCQAHGLTPEQVVAIASNIGGKQALETVQALL |
| | | PVLCQAHGLTPQQVVAIASNGGGKQALETVQRLLPVLCQAHGLTPEQVVAIAS |
| | | HDGGKQALETVQRLLPVLCQAHGLTPQQVVAIASNGGGRPALESIVAQLSRPD |
| | | PALAALTNDHLVALACLGGRPALDAVKKGLGDPISRSQLVKSELEEKKSELRH |
| | | KLKYVPHEYIELIEIARNSTQDRILEMKVMEFFMKVYGYRGKHLGGSRKPDGA |
| | | IYTVGSPIDYGVIVDTKAYSGGYNLPIGQADEMQRYVEENQTRNKHINPNEWW |
| | | KVYPSSVTEFKFLFVSGHFKGNYKAQLTRLNHITNCNGAVLSVEELLIGGEMI |
| | | KAGTLTLEEVRRKFNNGEINFAAD |
| 70 | TRAC | MGDPKKKRKVIDIADLRTLGYSQQQQEKIKPKVRSTVAQHHEALVGHGFTHAH |
| | TALEN right | IVALSQHPAALGTVAVKYQDMIAALPEATHEAIVGVGKQWSGARALEALLTVA |
| | polypeptide | GELRGPPLQLDTGQLLKIAKRGGVTAVEAVHAWRNALTGAPLNLTPEQVVAIA |
| | | SHDGGKQALETVQRLLPVLCQAHGLTPQQVVAIASNGGGKQALETVQRLLPVL |
| | | CQAHGLTPEQVVAIASHDGGKQALETVQRLLPVLCQAHGLTPEQVVAIASNIG |
| | | GKQALETVQALLPVLCQAHGLTPQQVVAIASNNGGKQALETVQRLLPVLCQAH |
| | | GLTPEQVVAIASHDGGKQALETVQRLLPVLCQAHGLTPQQVVAIASNGGGKQA |
| | | LETVQRLLPVLCQAHGLTPQQVVAIASNNGGKQALETVQRLLPVLCQAHGLTP |
| | | QQVVAIASNNGGKQALETVQRLLPVLCQAHGLTPQQVVAIASNGGGKQALETV |
| | | QRLLPVLCQAHGLTPEQVVAIASNIGGKQALETVQALLPVLCQAHGLTPEQVV |
| | | AIASHDGGKQALETVQRLLPVLCQAHGLTPEQVVAIASNIGGKQALETVQALL |
| | | PVLCQAHGLTPEQVVAIASHDGGKQALETVQRLLPVLCQAHGLTPQQVVAIAS |
| | | NNGGKQALETVQRLLPVLCQAHGLTPQQVVAIASNGGGRPALESIVAQLSRPD |
| | | PALAALTNDHLVALACLGGRPALDAVKKGLGDPISRSQLVKSELEEKKSELRH |
| | | KLKYVPHEYIELIEIARNSTQDRILEMKVMEFFMKVYGYRGKHLGGSRKPDGA |
| | | IYTVGSPIDYGVIVDTKAYSGGYNLPIGQADEMQRYVEENQTRNKHINPNEWW |
| | | KVYPSSVTEFKFLFVSGHFKGNYKAQLTRLNHITNCNGAVLSVEELLIGGEMI |
| | | KAGTLTLEEVRRKFNNGEINFAAD |

1. A chimeric polypeptide comprising a first and second polypeptides, said first polypeptide encoding a chimeric antigen receptor (CAR) and said second polypeptide comprising a protease having a cleavage activity directed against the first polypeptide.

2. A chimeric polypeptide according to claim 1, wherein said protease activity has the effect of preventing presentation of the CAR polypeptide at the surface of an immune cell in which said chimeric polypeptide is produced (switch-off).

3. A chimeric polypeptide according to claim 2, wherein said protease activity is inhibited by a protease inhibitor (switch-on).

4. A chimeric polypeptide according to claim 1, wherein said protease activity allows the excision of said second polypeptide to release the first polypeptide to form a functional CAR (switch-on).

5. A chimeric polypeptide according to claim 4, wherein said protease activity is inhibited by a protease inhibitor (switch-off).

6. A chimeric polypeptide according to claim 3 or 5, wherein said protease and said protease inhibitor are selected from the list of Table 2.

7. A chimeric polypeptide according to claim 3 or 5, wherein said protease inhibitor is a small molecule, such as simeprevir, danoprevir, asunaprevir and ciluprevir.

8. A chimeric polypeptide according to claim 7, wherein said protease shares identity with nonstructural protein 3 (NS3) protease.

9. A chimeric polypeptide according to any one of claims 1 to 8, wherein said chimeric polypeptide further comprises a degron polypeptide sequence, which enhances intracellular degradation of said chimeric polypeptide.

10. A chimeric polypeptide according to claim 9, wherein said degron comprises SEQ ID NO.32, 38, 41 or 43.

11. A chimeric polypeptide according to claim 9, wherein said degron is comprised into the sequence of said second polypeptide.

12. A chimeric polypeptide according to any one of claims 1 to 11, wherein said first polypeptide comprises a transmembrane domain linked to an extra cellular ligand binding-domain comprising VH and VL from a monoclonal antibody.

13. A chimeric polypeptide according to any one of claims 1 to 12, wherein said transmembrane domain is from CD8α transmembrane domain.

14. A chimeric polypeptide according to any one of claims 1 to 13, wherein said first polypeptide comprises a cytoplasmic domain including a CD3 zeta signaling domain and a co-stimulatory domain from 4-1BB.

15. A chimeric polypeptide according to any one of claims 1 to 14, wherein said first polypeptide further comprises a hinge such as a CD8α hinge, IgG1 hinge or FcγRIIIα hinge.

16. A chimeric polypeptide according to any one of claims 1 to 15, wherein said first polypeptide is constitutive of a single-chain CAR or of a transmembrane subunit of a multi-chain CAR.

17. A chimeric polypeptide according to any one of claims 1 to 16, wherein said CAR targets an antigen selected from CD19, CD22, CD33, CD38, CD123, CS1, CLL1, ROR1, OGD2, BCMA, HSP70 and EGFRvIII.

18. A polynucleotide encoding a chimeric polypeptide according to any one of claims 1 to 17.

19. A vector comprising a polynucleotide according to claim 18.

20. A set of polynucleotide sequences encoding respectively a first and second polypeptides, said first polypeptide encoding a chimeric antigen receptor (CAR) and said second polypeptide encoding a protease, said protease having a cleavage activity directed against the first polypeptide.

21. A set of polynucleotide sequences according to claim 20, wherein said sequences are borne on the same polynucleotide.

22. A set of polynucleotide sequences according to claim 21, wherein said sequences encode a chimeric polypeptide according to claim 1 to 17.

23. A set of polynucleotide sequences according to any one of claims 20 to 22, wherein said polynucleotide sequences are co-transfected into an immune cell.

24. An engineered immune cell transformed with a set of polynucleotide sequences according to any one of claims 20 to 23.

25. An engineered immune cell transformed with a polynucleotide encoding a chimeric polypeptide that comprises an effector polypeptide, a protease domain, and a degron.

26. An engineered immune cell transformed with a polynucleotide according to claim 18.

27. An engineered immune cell according to any one of claims 24 to 26, wherein said immune cell is a primary cell.

28. An engineered immune cell according to any one of claims 24 to 27, wherein said cell is a T-cell or a NK cell.

29. An engineered immune cell according to any one of claims 24 to 28, wherein the expression of TCR is reduced or suppressed in said effector immune cell.

30. An engineered immune cell according to any one of claims 24 to 29, wherein said CAR is encoded by an exogenous coding sequence introduced at a TCR locus.

31. An engineered immune cell according to any one of claims 24 to 30, wherein expression of at least one MHC protein, preferably β2m or HLA, is suppressed in said immune cell.

32. An engineered immune cell according to any one of claims 24 to 31, wherein said immune cell is provided from a donor or a patient.

33. An engineered immune cell according to any one of claims 24 to 32, for use in the treatment of cancer.

34. A method for inactivating (switching-off) a function linked to a transmembrane receptor into an effector cell, comprising at least the following steps:
providing an effector cell,
introducing into an effector cell a polynucleotide, or set of polynucleotide sequences encoding a chimeric polypeptide comprising a receptor polypeptide, a protease, and a degron;
expressing said chimeric polypeptide into said cell so that the protease activity removes the degron and said receptor polypeptide is presented at the surface of the cell;
introducing a protease inhibitor into the cell's environment, which inhibits said protease activity; such that the degron is not removed anymore and said expressed chimeric polypeptide is degraded by the proteasome, thereby switching off the function linked to the transmembrane receptor in said effector cell.

35. A method according to claim 34, wherein said chimeric polypeptide is according to claim 9.

36. A method for activating (switching-on) a function linked to a transmembrane receptor into an effector cell, comprising at least the following steps:
providing an effector cell,
introducing into said effector cell a set of polynucleotide sequences or a unique polynucleotide encoding (i) a transmembrane receptor polypeptide and (ii) a protease domain that is directed against said transmembrane receptor polypeptide,
expressing into said effector cell said polypeptides, the protease activity of which inactivates said receptor polypeptide function,
introducing a protease inhibitor in the immune cell's environment, in order to inhibit said protease activity and allow the transmembrane receptor to be presented at the cell surface, thereby activating the function of said receptor into said effector cell.

37. The method according to claim 36, wherein said polynucleotide sequences encoding (i) a transmembrane receptor polypeptide and (ii) a protease domain that is directed against said transmembrane receptor polypeptide are preferably separated by IRES (Internal Ribosome Entry Site) or a 2A peptide.

38. A method according to any one of claims 34 to 37, wherein said transmembrane receptor is a CAR.

39. A method according to any one of claims 34 to 37, wherein said transmembrane receptor is a recombinant TCR.

40. A method according to any one of claims 34 to 39, wherein said effector immune cell is a primary cell.

41. A method according to any one of claims 34 to 39, wherein said immune cell is a T-cell or a NK cell.

42. A method according to any one of claims 34 to 41, wherein expression of TCR is reduced or suppressed in said effector immune cell.

43. A method according to any one of claims 34 to 42, wherein expression of at least one MHC protein, preferably β2m or HLA, is suppressed in said immune cell.

44. A method according to anyone of claim 34 to 43, wherein said immune cell is provided from a donor or a patient.

45. A method according to any one of claims 34 to 43, for the treatment of a disease, wherein said effector immune cell endowed with the transmembrane receptor polypeptide contributes to eliminate pathological cells.

46. A method according to claim 45, wherein said transmembrane receptor polypeptide binds said pathological cells.

47. A method according to claim 46, wherein said pathological cells are malignant cells.


SEQUENCE LISTING (70 sequences)

SEQ ID NO: 1 - protein, 21 aa, artificial sequence
  CD8a signal peptide
MALPVTALLLPLALLLHAARP

SEQ ID NO: 2 - protein, 20 aa, artificial sequence
  Alternative signal peptide
METDTLLLWVLLLWVPGSTG

SEQ ID NO: 3 - protein, 16 aa, artificial sequence
  FcgammaRIIIalpha hinge
GLAVSTISSFFPPGYQ

SEQ ID NO: 4 - protein, 45 aa, artificial sequence
  CD8a hinge
TTTPAPRPPTPAPTIASQPLSLRPEACRPAAGGAVHTRGLDFACD

SEQ ID NO: 5 - protein, 231 aa, artificial sequence
  IgG1 hinge
EPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 24 aa, artificial sequence
  CD8a  transmembrane domain
IYIWAPLAGTCGVLLLSLVITLYC

SEQ ID NO: 7 - protein, 27 aa, artificial sequence
  41BB transmembrane domain
IISFFLALTSTALLFLLFFLTLRFSVV

SEQ ID NO: 8 - protein, 42 aa, artificial sequence
  41BB intracellular domain
KRGRKKLLYIFKQPFMRPVQTTQEEDGCSCRFPEEEEGGCEL

SEQ ID NO: 9 - protein, 112 aa, artificial sequence
  CD3 zeta intracellular domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 15 aa, artificial sequence
  G4Sx3 linker
GGGGSGGGGSGGGGS

SEQ ID NO: 11 - protein, 9 aa, artificial sequence
  Mimotope Rituximab
CPYSNPSLC

SEQ ID NO: 12 - protein, 24 aa, artificial sequence
  Epitope Palivizumab
NSELLSLINDMPITNDQKKLMSNN

SEQ ID NO: 13 - protein, 12 aa, artificial sequence
  Mimotope 1 Cetuximab
CQFDLSTRRLKC

SEQ ID NO: 14 - protein, 12 aa, artificial sequence
  Mimotope 2 Cetuximab
CQYNLSSRALKC

SEQ ID NO: 15 - protein, 12 aa, artificial sequence
  Mimotope 3 Cetuximab
CVWQRWQKSYVC

SEQ ID NO: 16 - protein, 12 aa, artificial sequence
  Mimotope 4 Cetuximab
CMWDRFSRWYKC

SEQ ID NO: 17 - protein, 25 aa, artificial sequence
  Epitope 1  Nivolumab
SFVLNWYRMSPSNQTDKLAAFPEDR

SEQ ID NO: 18 - protein, 19 aa, artificial sequence
  Epitope 2  Nivolumab
SGTYLCGAISLAPKAQIKE

SEQ ID NO: 19 - protein, 24 aa, artificial sequence
  Epitope QBEND-10
ELPTQGTFSNVSTNVSPAKPTTTA

SEQ ID NO: 20 - protein, 12 aa, artificial sequence
  Epitope Alemtuzumab
GQNDTSQTSSPS

SEQ ID NO: 21 - protein, 21 aa, artificial sequence
  CD8 signal sequence
MALPVTALLLPLALLLHAARP

SEQ ID NO: 22 - protein, 281 aa, artificial sequence
  CD123 targeting scFv
QIQLVQSGPELKKPGETVKISCKASGYIFTNYGMNWVKQAPGKSFKWMGWINTYTGESTYSADFKGRFAFSLETSASTAYLHINDLKNEDTATYFCARSGGYDPMDYWGQGTSVTVSSGGGGSGGGGSGGGGSDIVLTQSPASLAVSLGQRATISCRASESVDNYGNTFMHWYQQKPGQPPKLLIYRASNLESGIPARFSGSGSRTDFTLTINPVEADDVATYYCQQSNEDPPTFGAGTKLELKRSDPGSGGGGSCPYSNPSLCSGGGGSCPYSNPSLCAP

SEQ ID NO: 23 - protein, 248 aa, artificial sequence
  CD22 targeting scFv
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAP

SEQ ID NO: 24 - protein, 45 aa, artificial sequence; identical to SEQ ID NO: 4
  cd8 hinge

SEQ ID NO: 25 - protein, 24 aa, artificial sequence
  cd8 transmembrane
IYIWAPLAGTCGVLLLSLVITLYC

SEQ ID NO: 26 - protein, 154 aa, artificial sequence
  intracellular domain
RRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - protein, 41 aa, artificial sequence
  4-1BB costimulation domain
RGRKKLLYIFKQPFMRPVQTTQEEDGCSCRFPEEEEGGCEL

SEQ ID NO: 28 - protein, 112 aa, artificial sequence; identical to SEQ ID NO: 9
  CD3 activation domain

SEQ ID NO: 29 - protein, 10 aa, artificial sequence
  NS3 protease target site
DEMEECSQHL

SEQ ID NO: 30 - protein, 30 aa, artificial sequence
  linker/tag
PGAGSSGDIMDYKDDDDKGSSGTGSGSGTS

SEQ ID NO: 31 - protein, 186 aa, artificial sequence
  NS3 Protease domain
APITAYAQQTRGLLGCIITSLTGRDKNQVEGEVQIVSTATQTFLATCINGVCWAVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTD

SEQ ID NO: 32 - protein, 78 aa, artificial sequence
  NS3/NS4 degron
NSSPPAVTLTHPITKIDTKYIMTCMSADLEVVTSTWVLVGGVLAALAAYCLSTGCVVIVGRIVLSGKPAIIPDREVLY

SEQ ID NO: 33 - protein, 832 aa, artificial sequence
  pCLS29306 (Chimeric polypeptide targeting CD123)
MALPVTALLLPLALLLHAARPQIQLVQSGPELKKPGETVKISCKASGYIFTNYGMNWVKQAPGKSFKWMGWINTYTGESTYSADFKGRFAFSLETSASTAYLHINDLKNEDTATYFCARSGGYDPMDYWGQGTSVTVSSGGGGSGGGGSGGGGSDIVLTQSPASLAVSLGQRATISCRASESVDNYGNTFMHWYQQKPGQPPKLLIYRASNLESGIPARFSGSGSRTDFTLTINPVEADDVATYYCQQSNEDPPTFGAGTKLELKRSDPGSGGGGSCPYSNPSLCSGGGGSCPYSNPSLCAPTTTPAPRPPTPAPTIASQPLSLRPEACRPAAGGAVHTRGLDFACDIYIWAPLAGTCGVLLLSLVITLYCRRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGDEMEECSQHLPGAGSSGDIMDYKDDDDKGSSGTGSGSGTSAPITAYAQQTRGLLGCIITSLTGRDKNQVEGEVQIVSTATQTFLATCINGVCWAVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTDNSSPPAVTLTHPITKIDTKYIMTCMSADLEVVTSTWVLVGGVLAALAAYCLSTGCVVIVGRIVLSGKPAIIPDREVLYE

SEQ ID NO: 34 - protein, 798 aa, artificial sequence
  pCLS30066 (Chimeric polypeptide targeting CD22)
MALPVTALLLPLALLLHAARPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAPTTTPAPRPPTPAPTIASQPLSLRPEACRPAAGGAVHTRGLDFACDIYIWAPLAGTCGVLLLSLVITLYCRRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGDEMEECSQHLPGAGSSGDIMDYKDDDDKGSSGTGSGSGTSAPITAYAQQTRGLLGCIITSLTGRDKNQVEGEVQIVSTATQTFLATCINGVCWAVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTDNSSPPAVTLTHPITKIDTKYIMTCMSADLEVVTSTWVLVGGVLAALAAYCLSTGCVVIVGRIVLSGKPAIIPDREVLY

SEQ ID NO: 35 - DNA, 492 bp, artificial sequence
  SFFV promoter
gataaaataaaagattttatttagtctccagaaaaaggggggaatgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccggggg

SEQ ID NO: 36 - protein, 21 aa, artificial sequence
  linker/tag Nter fusion
MDYKDDDDKGSSGTGSGSGTS

SEQ ID NO: 37 - protein, 186 aa, artificial sequence; identical to SEQ ID NO: 31
  NS3 protease domain Nter

SEQ ID NO: 38 - protein, 87 aa, artificial sequence
  NS3/NS4 degron Nter
NSSPPAVTLTHPITKIDTKYIMTCMSADLEVVTSTWVLVGGVLAALAAYCLSTGCVVIVGRIVLSGKPAGSSGSSIIPDREVLYQEF

SEQ ID NO: 39 - protein, 9 aa, artificial sequence
  NS3 protease target site Nter
EDVVPCSMG

SEQ ID NO: 40 - protein, 799 aa, artificial sequence
  Polypeptide from pCLS30018
MDYKDDDDKGSSGTGSGSGTSAPITAYAQQTRGLLGCIITSLTGRDKNQVEGEVQIVSTATQTFLATCINGVCWAVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTDNSSPPAVTLTHPITKIDTKYIMTCMSADLEVVTSTWVLVGGVLAALAAYCLSTGCVVIVGRIVLSGKPAGSSGSSIIPDREVLYQEFEDVVPCSMGSGAPMALPVTALLLPLALLLHAARPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAPTTTPAPRPPTPAPTIASQPLSLRPEACRPAAGGAVHTRGLDFACDIYIWAPLAGTCGVLLLSLVITLYCRRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 41 - protein, 38 aa, artificial sequence
  IKB based degron
VNRVTYQGYSPYQLTWGRPSTRIQQQLGQLTLENLQML

SEQ ID NO: 42 - DNA, 2304 bp, artificial sequence
  pCLS30575 (CD22-degron-IKB, part of NF-kappa-B inhibitor alpha, Gene NFKBIA)
atggctctgcccgtcaccgctctgctgctgccactggcactgctgctgcacgctgctaggccccaggtgcagctgcagcagagcggccctggcctggtgaagccaagccagacactgtccctgacctgcgccatcagcggcgattccgtgagctccaactccgccgcctggaattggatcaggcagtccccttctcggggcctggagtggctgggaaggacatactatcggtctaagtggtacaacgattatgccgtgtctgtgaagagcagaatcacaatcaaccctgacacctccaagaatcagttctctctgcagctgaatagcgtgacaccagaggacaccgccgtgtactattgcgccagggaggtgaccggcgacctggaggatgcctttgacatctggggccagggcacaatggtgaccgtgtctagcggaggcggaggctccggaggcggaggatctggcggaggcggaagcgatatccagatgacacagtccccatcctctctgagcgcctccgtgggcgacagagtgacaatcacctgtagggcctcccagaccatctggtcttacctgaactggtatcagcagaggcccggcaaggcccctaatctgctgatctacgcagcaagctccctgcagagcggagtgccatccagattctctggcaggggctccggcacagacttcaccctgaccatctctagcctccaggccgaggacttcgccacctactattgccagcagtcttatagcatcccccagacatttggccagggcaccaagctggagatcaaggctcccaccacaacccccgctccaaggccccctacccccgcaccaactattgcctcccagccactctcactgcggcctgaggcctgtcggcccgctgctggaggcgcagtgcatacaaggggcctcgatttcgcctgcgatatttacatctgggcacccctcgccggcacctgcggggtgcttctcctctccctggtgattaccctgtattgcagacggggccggaagaagctcctctacatttttaagcagcctttcatgcggccagtgcagacaacccaagaggaggatgggtgttcctgcagattccctgaggaagaggaaggcgggtgcgagctgagagtgaagttctccaggagcgcagatgcccccgcctatcaacagggccagaaccagctctacaacgagcttaacctcgggaggcgcgaagaatacgacgtgttggataagagaagggggcgggaccccgagatgggaggaaagccccggaggaagaaccctcaggagggcctgtacaacgagctgcagaaggataagatggccgaggcctactcagagatcgggatgaagggggagcggcgccgcgggaaggggcacgatgggctctaccaggggctgagcacagccacaaaggacacatacgacgccttgcacatgcaggcccttccaccccggtctggagatgagatggaagagtgctctcagcacttacccggcgccggcagtagtggcgatatcatggattacaaggatgacgacgataagggctcttccgggacaggctccggatccggcactagtgcgcccatcacggcgtacgcccagcagacgagaggcctcctagggtgtataatcaccagcctgactggccgggacaaaaaccaagtggagggtgaggtccagatcgtgtcaactgctacccaaaccttcctggcaacgtgcatcaatggggtatgctgggcagtctaccacggggccggaacgaggaccatcgcatcacccaagggtcctgtcatccagatgtataccaatgtggaccaagaccttgtgggctggcccgctcctcaaggttcccgctcattgacaccctgtacctgcggctcctcggacctttacctggtcacgaggcacgccgatgtcattcccgtgcgccggcgaggtgatagcaggggtagcctgctttcgccccggcccatttcctacttgaaaggctcctctgggggtccgctgttgtgccccgcgggacacgccgtgggcctattcagggccgcggtgtgcacccgtggagtggctaaagcggtggactttatccctgtggagaacctagagacaaccatgagatccccggtgttcacggacaactcctctccaccagcagtcaccctgacggtgaacagggtgacctaccagggctacagcccctaccagctgacctggggcaggcccagcaccaggatccagcagcagctgggccagctgaccctggagaacctgcagatgctg

SEQ ID NO: 43 - protein, 15 aa, artificial sequence
  SMNd7 based degron
YMSGYHTGYYMEMLA

SEQ ID NO: 44 - DNA, 2235 bp, artificial sequence
  pCLS30576 (CD22-degron-SMNd7)
atggctctgcccgtcaccgctctgctgctgccactggcactgctgctgcacgctgctaggccccaggtgcagctgcagcagagcggccctggcctggtgaagccaagccagacactgtccctgacctgcgccatcagcggcgattccgtgagctccaactccgccgcctggaattggatcaggcagtccccttctcggggcctggagtggctgggaaggacatactatcggtctaagtggtacaacgattatgccgtgtctgtgaagagcagaatcacaatcaaccctgacacctccaagaatcagttctctctgcagctgaatagcgtgacaccagaggacaccgccgtgtactattgcgccagggaggtgaccggcgacctggaggatgcctttgacatctggggccagggcacaatggtgaccgtgtctagcggaggcggaggctccggaggcggaggatctggcggaggcggaagcgatatccagatgacacagtccccatcctctctgagcgcctccgtgggcgacagagtgacaatcacctgtagggcctcccagaccatctggtcttacctgaactggtatcagcagaggcccggcaaggcccctaatctgctgatctacgcagcaagctccctgcagagcggagtgccatccagattctctggcaggggctccggcacagacttcaccctgaccatctctagcctccaggccgaggacttcgccacctactattgccagcagtcttatagcatcccccagacatttggccagggcaccaagctggagatcaaggctcccaccacaacccccgctccaaggccccctacccccgcaccaactattgcctcccagccactctcactgcggcctgaggcctgtcggcccgctgctggaggcgcagtgcatacaaggggcctcgatttcgcctgcgatatttacatctgggcacccctcgccggcacctgcggggtgcttctcctctccctggtgattaccctgtattgcagacggggccggaagaagctcctctacatttttaagcagcctttcatgcggccagtgcagacaacccaagaggaggatgggtgttcctgcagattccctgaggaagaggaaggcgggtgcgagctgagagtgaagttctccaggagcgcagatgcccccgcctatcaacagggccagaaccagctctacaacgagcttaacctcgggaggcgcgaagaatacgacgtgttggataagagaagggggcgggaccccgagatgggaggaaagccccggaggaagaaccctcaggagggcctgtacaacgagctgcagaaggataagatggccgaggcctactcagagatcgggatgaagggggagcggcgccgcgggaaggggcacgatgggctctaccaggggctgagcacagccacaaaggacacatacgacgccttgcacatgcaggcccttccaccccggtctggagatgagatggaagagtgctctcagcacttacccggcgccggcagtagtggcgatatcatggattacaaggatgacgacgataagggctcttccgggacaggctccggatccggcactagtgcgcccatcacggcgtacgcccagcagacgagaggcctcctagggtgtataatcaccagcctgactggccgggacaaaaaccaagtggagggtgaggtccagatcgtgtcaactgctacccaaaccttcctggcaacgtgcatcaatggggtatgctgggcagtctaccacggggccggaacgaggaccatcgcatcacccaagggtcctgtcatccagatgtataccaatgtggaccaagaccttgtgggctggcccgctcctcaaggttcccgctcattgacaccctgtacctgcggctcctcggacctttacctggtcacgaggcacgccgatgtcattcccgtgcgccggcgaggtgatagcaggggtagcctgctttcgccccggcccatttcctacttgaaaggctcctctgggggtccgctgttgtgccccgcgggacacgccgtgggcctattcagggccgcggtgtgcacccgtggagtggctaaagcggtggactttatccctgtggagaacctagagacaaccatgagatccccggtgttcacggacaactcctctccaccagcagtcaccctgacgtacatgagcggctaccacaccggctactacatggagatgctggcc

SEQ ID NO: 45 - protein, 4 aa, Artificial Sequence
  Linker
SGGS

SEQ ID NO: 46 - protein, 5 aa, Artificial Sequence
  Linker
SSGGS

SEQ ID NO: 47 - protein, 4 aa, Artificial Sequence
  Linker
GGGG

SEQ ID NO: 48 - protein, 5 aa, Artificial Sequence
  Linker
SGGGG

SEQ ID NO: 49 - protein, 5 aa, Artificial Sequence
  Linker
GGGGS

SEQ ID NO: 50 - protein, 6 aa, Artificial Sequence
  Linker
SGGGGS

SEQ ID NO: 51 - protein, 6 aa, Artificial Sequence
  Linker
GGGGGS

SEQ ID NO: 52 - protein, 7 aa, Artificial Sequence
  Linker
SGGGGGS

SEQ ID NO: 53 - protein, 6 aa, Artificial Sequence
  Linker
SGGGGG

SEQ ID NO: 54 - protein, 7 aa, Artificial Sequence
  Linker
GSGGGGS

SEQ ID NO: 55 - protein, 8 aa, Artificial Sequence
  Linker
GGGGGGGS

SEQ ID NO: 56 - protein, 8 aa, Artificial Sequence
  Linker
SGGGGGGG

SEQ ID NO: 57 - protein, 9 aa, Artificial Sequence
  Linker
SGGGGGGGS

SEQ ID NO: 58 - protein, 11 aa, Artificial Sequence
  Linker
SGGGGSGGGGS

SEQ ID NO: 59 - DNA, 3363 bp, artificial sequence
  integration matrix
aagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagtacccctacgacgtgcccgactacgcctccggtgagggcagaggaagtcttctaacatgcggtgacgtggaggagaatccgggccccggatccgctctgcccgtcaccgctctgctgctgccactggcactgctgctgcacgctgctaggccccaggtgcagctgcagcagagcggccctggcctggtgaagccaagccagacactgtccctgacctgcgccatcagcggcgattccgtgagctccaactccgccgcctggaattggatcaggcagtccccttctcggggcctggagtggctgggaaggacatactatcggtctaagtggtacaacgattatgccgtgtctgtgaagagcagaatcacaatcaaccctgacacctccaagaatcagttctctctgcagctgaatagcgtgacaccagaggacaccgccgtgtactattgcgccagggaggtgaccggcgacctggaggatgcctttgacatctggggccagggcacaatggtgaccgtgtctagcggaggcggaggctccggaggcggaggatctggcggaggcggaagcgatatccagatgacacagtccccatcctctctgagcgcctccgtgggcgacagagtgacaatcacctgtagggcctcccagaccatctggtcttacctgaactggtatcagcagaggcccggcaaggcccctaatctgctgatctacgcagcaagctccctgcagagcggagtgccatccagattctctggcaggggctccggcacagacttcaccctgaccatctctagcctccaggccgaggacttcgccacctactattgccagcagtcttatagcatcccccagacatttggccagggcaccaagctggagatcaaggctcccaccacaacccccgctccaaggccccctacccccgcaccaactattgcctcccagccactctcactgcggcctgaggcctgtcggcccgctgctggaggcgcagtgcatacaaggggcctcgatttcgcctgcgatatttacatctgggcacccctcgccggcacctgcggggtgcttctcctctccctggtgattaccctgtattgcagacggggccggaagaagctcctctacatttttaagcagcctttcatgcggccagtgcagacaacccaagaggaggatgggtgttcctgcagattccctgaggaagaggaaggcgggtgcgagctgagagtgaagttctccaggagcgcagatgcccccgcctatcaacagggccagaaccagctctacaacgagcttaacctcgggaggcgcgaagaatacgacgtgttggataagagaagggggcgggaccccgagatgggaggaaagccccggaggaagaaccctcaggagggcctgtacaacgagctgcagaaggataagatggccgaggcctactcagagatcgggatgaagggggagcggcgccgcgggaaggggcacgatgggctctaccaggggctgagcacagccacaaaggacacatacgacgccttgcacatgcaggcccttccaccccggtctggagatgagatggaagagtgctctcagcacttacccggcgccggcagtagtggcgatatcatggattacaaggatgacgacgataagggctcttccgggacaggctccggatccggcactagtgcgcccatcacggcgtacgcccagcagacgagaggcctcctagggtgtataatcaccagcctgactggccgggacaaaaaccaagtggagggtgaggtccagatcgtgtcaactgctacccaaaccttcctggcaacgtgcatcaatggggtatgctgggcagtctaccacggggccggaacgaggaccatcgcatcacccaagggtcctgtcatccagatgtataccaatgtggaccaagaccttgtgggctggcccgctcctcaaggttcccgctcattgacaccctgtacctgcggctcctcggacctttacctggtcacgaggcacgccgatgtcattcccgtgcgccggcgaggtgatagcaggggtagcctgctttcgccccggcccatttcctacttgaaaggctcctctgggggtccgctgttgtgccccgcgggacacgccgtgggcctattcagggccgcggtgtgcacccgtggagtggctaaagcggtggactttatccctgtggagaacctagagacaaccatgagatccccggtgttcacggacaactcctctccaccagcagtcaccctgacgcacccaatcaccaaaatcgataccaaatacatcatgacatgcatgtcggccgacctggaggtcgtcacgagcacctgggtgctcgttggcggcgtcctggctgctctggccgcgtattgcctgtcaacaggctgcgtggtcatagtgggcaggatcgtcttgtccgggaagccggcaattatacctgacagggaggttctctactgatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgactagtggcgaattcccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaa

SEQ ID NO: 60 - DNA, 300 bp, artificial sequence
  TRAC left homology
aagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccag

SEQ ID NO: 61 - DNA, 27 bp, artificial sequence
  HA tag
tacccctacgacgtgcccgactacgcc

SEQ ID NO: 62 - DNA, 54 bp, artificial sequence
  2A element
gagggcagaggaagtcttctaacatgcggtgacgtggaggagaatccgggcccc

SEQ ID NO: 63 - DNA, 2394 bp, artificial sequence
  SWOFF anti CD22 CAR
gctctgcccgtcaccgctctgctgctgccactggcactgctgctgcacgctgctaggccccaggtgcagctgcagcagagcggccctggcctggtgaagccaagccagacactgtccctgacctgcgccatcagcggcgattccgtgagctccaactccgccgcctggaattggatcaggcagtccccttctcggggcctggagtggctgggaaggacatactatcggtctaagtggtacaacgattatgccgtgtctgtgaagagcagaatcacaatcaaccctgacacctccaagaatcagttctctctgcagctgaatagcgtgacaccagaggacaccgccgtgtactattgcgccagggaggtgaccggcgacctggaggatgcctttgacatctggggccagggcacaatggtgaccgtgtctagcggaggcggaggctccggaggcggaggatctggcggaggcggaagcgatatccagatgacacagtccccatcctctctgagcgcctccgtgggcgacagagtgacaatcacctgtagggcctcccagaccatctggtcttacctgaactggtatcagcagaggcccggcaaggcccctaatctgctgatctacgcagcaagctccctgcagagcggagtgccatccagattctctggcaggggctccggcacagacttcaccctgaccatctctagcctccaggccgaggacttcgccacctactattgccagcagtcttatagcatcccccagacatttggccagggcaccaagctggagatcaaggctcccaccacaacccccgctccaaggccccctacccccgcaccaactattgcctcccagccactctcactgcggcctgaggcctgtcggcccgctgctggaggcgcagtgcatacaaggggcctcgatttcgcctgcgatatttacatctgggcacccctcgccggcacctgcggggtgcttctcctctccctggtgattaccctgtattgcagacggggccggaagaagctcctctacatttttaagcagcctttcatgcggccagtgcagacaacccaagaggaggatgggtgttcctgcagattccctgaggaagaggaaggcgggtgcgagctgagagtgaagttctccaggagcgcagatgcccccgcctatcaacagggccagaaccagctctacaacgagcttaacctcgggaggcgcgaagaatacgacgtgttggataagagaagggggcgggaccccgagatgggaggaaagccccggaggaagaaccctcaggagggcctgtacaacgagctgcagaaggataagatggccgaggcctactcagagatcgggatgaagggggagcggcgccgcgggaaggggcacgatgggctctaccaggggctgagcacagccacaaaggacacatacgacgccttgcacatgcaggcccttccaccccggtctggagatgagatggaagagtgctctcagcacttacccggcgccggcagtagtggcgatatcatggattacaaggatgacgacgataagggctcttccgggacaggctccggatccggcactagtgcgcccatcacggcgtacgcccagcagacgagaggcctcctagggtgtataatcaccagcctgactggccgggacaaaaaccaagtggagggtgaggtccagatcgtgtcaactgctacccaaaccttcctggcaacgtgcatcaatggggtatgctgggcagtctaccacggggccggaacgaggaccatcgcatcacccaagggtcctgtcatccagatgtataccaatgtggaccaagaccttgtgggctggcccgctcctcaaggttcccgctcattgacaccctgtacctgcggctcctcggacctttacctggtcacgaggcacgccgatgtcattcccgtgcgccggcgaggtgatagcaggggtagcctgctttcgccccggcccatttcctacttgaaaggctcctctgggggtccgctgttgtgccccgcgggacacgccgtgggcctattcagggccgcggtgtgcacccgtggagtggctaaagcggtggactttatccctgtggagaacctagagacaaccatgagatccccggtgttcacggacaactcctctccaccagcagtcaccctgacgcacccaatcaccaaaatcgataccaaatacatcatgacatgcatgtcggccgacctggaggtcgtcacgagcacctgggtgctcgttggcggcgtcctggctgctctggccgcgtattgcctgtcaacaggctgcgtggtcatagtgggcaggatcgtcttgtccgggaagccggcaattatacctgacagggaggttctctactga

SEQ ID NO: 64 - DNA, 228 bp, artificial sequence
  BGH poly A
cgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatga

SEQ ID NO: 65 - DNA, 290 bp, artificial sequence
  TRAC right homology
gctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaa

SEQ ID NO: 66 - DNA, 2781 bp, artificial sequence
  TRAC TALEN left
atgggcgatcctaaaaagaaacgtaaggtcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 67 - DNA, 2773 bp, artificial sequence
  TRAC TALEN right
atgggcgatcctaaaaagaaacgtaaggtcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccg

SEQ ID NO: 68 - protein, 797 aa, artificial sequence
  SWOFF anti CD22 CAR polypeptide
ALPVTALLLPLALLLHAARPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAPTTTPAPRPPTPAPTIASQPLSLRPEACRPAAGGAVHTRGLDFACDIYIWAPLAGTCGVLLLSLVITLYCRRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGDEMEECSQHLPGAGSSGDIMDYKDDDDKGSSGTGSGSGTSAPITAYAQQTRGLLGCIITSLTGRDKNQVEGEVQIVSTATQTFLATCINGVCWAVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTDNSSPPAVTLTHPITKIDTKYIMTCMSADLEVVTSTWVLVGGVLAALAAYCLSTGCVVIVGRIVLSGKPAIIPDREVLY

SEQ ID NO: 69 - protein, 925 aa, artificial sequence
  TRAC TALEN left polypeptide
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 70 - protein, 925 aa, artificial sequence
  TRAC TALEN right polypeptide
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNNGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD